(19) United States
(12) Patent Application Publication
(10) Pub. No.: US 2012/0041012 A1
(43) Pub. Date: Feb. 16, 2012

(54) SUBSTITUTED SPIROCYCLIC AMINES USEFUL AS ANTIDIABETIC COMPOUNDS

(76) Inventors: Susan D. Aster, Teaneck, NJ (US); Joseph L. Duffy, Cranford, NJ (US); Gui-Bai Liang, Scotch Plains, NJ (US); Patrick Shao, Fanwood, NJ (US); Feng Ye, Scotch Plains, NJ (US)

(21) Appl. No.: 13/265,146

(22) PCT Filed: May 6, 2010
(86) PCT No.: PCT/US2010/033808

§ 371 Date: Oct. 19, 2011

Related U.S. Application Data
(60) Provisional application No. 61/215,591, filed on May 7, 2009.
(60) Provisional application No. 61/250,902, filed on Oct. 13, 2009.

(51) Int. Cl. A61K 31/438; A61K 31/506; A61P 3/10; A61P 25/22; A61P 3/06; A61P 3/04; A61P 3/00; A61P 25/24; C07D 471/10; A61P 5/48

(52) U.S. Cl. 514/275; 546/16; 514/278; 544/230

(57) ABSTRACT

Substituted spirocyclic amines of structural formula I are selective antagonists of the somatostatin sub-type receptor 5 (SSTR5) and are useful for the treatment, control or prevention of disorders responsive to antagonism of SSTR5, such as Type 2 diabetes, insulin resistance, lipid disorders, obesity, atherosclerosis, metabolic syndrome, depression, and anxiety.

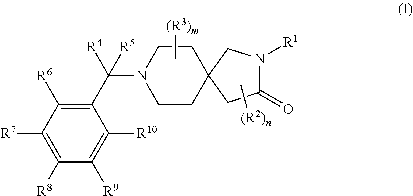

### FIELD OF THE INVENTION

The instant invention is concerned with substituted spirocyclic amines, which are selective antagonists of the somatostatin subtype receptor 5 (SSTR5) and are useful for the treatment, control or prevention of disorders responsive to antagonism of SSTR5, such as of Type 2 diabetes mellitus, insulin resistance, obesity, lipid disorders, atherosclerosis, metabolic syndrome, depression, and anxiety.

### BACKGROUND OF THE INVENTION

Diabetes is a disease derived from multiple causative factors and characterized by elevated levels of plasma glucose (hyperglycemia) in the fasting state or after administration of glucose during an oral glucose tolerance test. There are two generally recognized fauns of diabetes. In type 1 diabetes, or insulin-dependent diabetes mellitus (IDDM), patients produce little or no insulin, the hormone which regulates glucose utilization. In Type 2 diabetes, or noninsulin-dependent diabetes mellitus (NIDDM), insulin is still produced by islet cells in the pancreas. Patients having Type 2 diabetes have a resistance to the effects of insulin in stimulating glucose and lipid metabolism in the main insulin-sensitive tissues, including muscle, liver and adipose tissues. These patients often have normal levels of insulin, and may have hyperinsulinemia (elevated plasma insulin levels), as they compensate for the reduced effectiveness of insulin by secreting increased amounts of insulin (Polonsky, Int. J. Obes. Relat. Metab. Disord. 24 Suppl 2:S29-31, 2000). The beta cells within the pancreatic islets initially compensate for insulin resistance by increasing insulin output. Insulin resistance is not primarily caused by a diminished number of insulin receptors but rather by a post-insulin receptor binding defect that is not yet completely understood. This lack of responsiveness to insulin results in insufficient insulin-mediated activation of uptake, oxidation and storage of glucose in muscle, and inadequate insulin-mediated repression of lipolysis in adipose tissue and of glucose production and secretion in the liver. Eventually, a patient may be become diabetic due to the inability to properly compensate for insulin resistance. In humans, the onset of Type 2 diabetes due to insufficient increases (or actual declines) in beta cell mass is apparently due to increased beta cell apoptosis relative to non-diabetic insulin resistant individuals (Butler et al., Diabetes 52:102-110, 2003).

Persistent or uncontrolled hyperglycemia that occurs with diabetes is associated with increased and premature morbidity and mortality. Often abnormal glucose homeostasis is associated both directly and indirectly with obesity, hypertension, and alterations of the lipid, lipoprotein and apolipoprotein metabolism, as well as other metabolic and hemodynamic disease. Patients with Type 2 diabetes mellitus have a significantly increased risk of macrovascular and microvascular complications, including atherosclerosis, coronary heart disease, stroke, peripheral vascular disease, hypertension, nephropathy, neuropathy, and retinopathy. Therefore, effective therapeutic control of glucose homeostasis, lipid metabolism, obesity, and hypertension are critically important in the clinical management and treatment of diabetes mellitus.

Patients who have insulin resistance often exhibit several symptoms that together are referred to as syndrome X or Metabolic Syndrome. According to one widely used definition, a patient having Metabolic Syndrome is characterized as having three or more symptoms selected from the following group of five symptoms: (1) abdominal obesity, (2) hypertriglyceridemia, (3) low levels of high-density lipoprotein cholesterol (HDL), (4) high blood pressure, and (5) elevated fasting glucose, which may be in the range characteristic of Type 2 diabetes if the patient is also diabetic. Each of these symptoms is defined clinically in the Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with Metabolic Syndrome, whether they have or develop overt diabetes mellitus, have an increased risk of developing the macrovascular and microvascular complications that occur with Type 2 diabetes, such as atherosclerosis and coronary heart disease.

There are several available treatments for Type 2 diabetes, each of which has its own limitations and potential risks. Physical exercise and a reduction in dietary intake of calories often dramatically improves the diabetic condition and are the usual recommended first-line treatment of Type 2 diabetes and of pre-diabetic conditions associated with insulin resistance. Compliance with this treatment is generally very poor because of well-entrenched sedentary lifestyles and excess food consumption, especially of foods containing high amounts of fat and carbohydrates. Pharmacologic treatments have largely focused on three areas of pathophysiology: (1) hepatic glucose production (biguanides such as phenfoimin and metformin), (2) insulin resistance (PPAR agonists such as rosiglitazone and pioglitazone), (3) insulin secretagogues (sulfonylureas such as tolbutamide, glipizide, and glimepiride); (4) incretin hormone mimetics (GLP-1 derivatives and analogs, such as exenatide and luraglitide); and (5) inhibitors of incretin hormone degradation (DPP-4 inhibitors, such as sitagliptin, vildagliptin, saxagliptin, and alogliptin).

Recent research has focused on pancreatic islet-based insulin secretion that is controlled by glucose-dependent insulin secretion. This approach has the potential for stabilization and restoration of β-cell function. In this regard, research has been done on the affects of antagonizing one or more of the somatostatin receptors. Somatostatin (SST) is a cyclic tetradecapeptide hormone that is widely distributed throughout the body and exhibits multiple biological functions that are mostly inhibitory in function, such as the release of growth hormone, pancreatic insulin, glucagon, and gastrin.

SST hormone activity is mediated through SST-14 and SST-28 isoforms that differentially bind to the five different SST receptor subtypes (SSTR1-5). In humans SSTR1 and SSTR2 are found in the pituitary, small intestine, heart and spleen with SSTR2 predominately in the pancreas, pituitary and the stomach. SSTR3 and SSTR4 are found in the pituitary, heart, liver, spleen stomach, small intestine and kidney. SSTR5 is found in high concentration in the pituitary, as well as the pancreas. It has been shown that 5-28 and S-14 bind with similar affinity to SSTR1, SSTR2, SSTR3, and SSTR4. The receptor SSTR5 can be characterized by its preferential affinity for 5-28 (Chisholm et al., Am. J. Physiol Endocrinol Metab. 283:E311-E317 (2002)).

SSTR5 is expressed by human islet β cells that are responsible for producing insulin and amylin. Therefore, binding to the SSTR5 could affect insulin secretion. For example, by using in vitro isolated perfused pancreas preparations from 3-month-old mice, it was demonstrated that SSTR5 global knockout mice pancreata have low basal insulin production, but a near normal response to glucose stimulation. It was theorized that, since along with SSTR5, SSTR1 is also expressed in islet β cells up-regulated SSTR1 compensates for the loss of SSTR5 in young knockout mice. As the mice aged, however, SSTR1 expression decreased in both the knockout mice and the aged-control wild-type mice. With lower SSTR1 expression in vivo, SSTR5 knockout mice had increased basal and glucose stimulated insulin secretion due to near complete lack of SSTRs on the knockout mice islet p cells with subsequent loss of the inhibitory SST response (Wang et al., Journal of Surgical Research, 129, 64-72 (2005)).

The proximity of D cells producing S-28 and L-cells containing GLP-1 in the ileum suggest that S-28 acting through SSTR5 may additionally participate in the direct regulation of GLP-1 secretion. To determine if S-28 acting through SSTR5 participates in the direct regulation of GLP-1 secretion, fetal rat intestinal cell cultures were treated with somatostatin analogs with relatively high specificity for SSTR2-5. GLP-1 secretion was inhibited by an SSTR5-selective analog more potently that S-14 and nearly as effectively as S-28 (Chisholm et al., Am. J. Physiol Endocrinol Metab. 283:E311-E317, 2002). A selective antagonist of SSTR5 is anticipated to block the suppression of GLP-1 secretion by endogenous somatostatin peptides, thereby elevating circulating GLP-1 levels. Elevated endogenous GLP-1 levels are associated with beneficial effects in the treatment of type 2 diabetes (Arulmozhi et al., European Journal of Pharmaceutical Sciences, 28, 96-108 (2006)).

US 2008/0293756 discloses 4,4 disubstituted piperidine derivatives as SST Receptor Subtype 5 antagonists useful to treat diabetes.

Small molecule SSTR antagonists are also disclosed in US 20080249101; WO 2008031735; WO 2008019967; WO 2006094682; WO 2006128803; WO 2007025897; WO 20070110340 and WO 2008000692.

Other small molecule and peptide SSTR antagonists known in the art are disclosed in Wilkinson et al., British Journal of Pharmacology 118, 445-447 (1996); Hocart et al., J. Med. Chem. 41, 1146-1154 (1998); Hay et al., Bioorg. Med. Chem. Lett 11, 2731-2734 (2001), Martin et al., J. Med. Chem. 50, 6291-6295 (2007) and Guba et al., J. Med. Chem. 50, 6295-6298 (2007).

Described herein are selective, directly acting SSTR5 antagonists, which are useful as therapeutically active agents for the treatment and/or prevention of diseases that are associated with the modulation of SSTR5. Diseases that can be treated or prevented with SSTR5 antagonists include diabetes mellitus, impaired glucose tolerance and elevated fasting glucose.

### SUMMARY OF THE INVENTION

The present invention is directed to compounds of structural formula I, and pharmaceutically acceptable salts thereof:

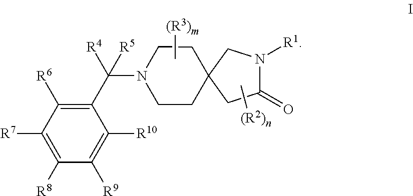

These substituted spirocyclic amines are effective as antagonists of SSTR5, and are useful for the treatment, control or prevention of disorders responsive to antagonism of SSTR5, such as type 2 diabetes, insulin resistance, lipid disorders, obesity, atherosclerosis, metabolic syndrome, depression, and anxiety.

The present invention also relates to pharmaceutical compositions comprising the compounds of the present invention and a pharmaceutically acceptable carrier.

The present invention also relates to methods for the treatment, control, or prevention of disorders, diseases, or conditions responsive to antagonism of SSTR5 in a subject in need thereof by administering the compounds and pharmaceutical compositions of the present invention.

The present invention also relates to methods for the treatment, control, or prevention of Type 2 diabetes, hyperglycemia, insulin resistance, obesity, lipid disorders, atherosclerosis, and metabolic syndrome by administering the compounds and pharmaceutical compositions of the present invention to a subject in need thereof.

The present invention also relates to methods for the treatment, control, or prevention of depression and anxiety by administering the compounds and pharmaceutical compositions of the present invention in a subject in need thereof.

The present invention also relates to methods of enhancing GLP-1 secretion by administering the compounds and pharmaceutical compositions of the present invention to a subject in need thereof.

The present invention also relates to methods for the treatment, control, or prevention of obesity by administering the compounds of the present invention in combination with a therapeutically effective amount of another agent known to be useful to treat obesity.

The present invention also relates to methods for the treatment, control, or prevention of Type 2 diabetes by administering the compounds of the present invention in combination with a therapeutically effective amount of another agent known to be useful to treat type 2 diabetes.

The present invention also relates to methods for the treatment, control, or prevention of atherosclerosis by administering the compounds of the present invention in combination with a therapeutically effective amount of another agent known to be useful to treat atherosclerosis.

The present invention also relates to methods for the treatment, control, or prevention of lipid disorders by administering the compounds of the present invention in combination with a therapeutically effective amount of another agent known to be useful to treat lipid disorders.

The present invention also relates to methods for treating metabolic syndrome by administering the compounds of the present invention in combination with a therapeutically effective amount of another agent known to be useful to treat metabolic syndrome.

The present invention also relates to methods for the treatment, control, or prevention of depression and anxiety by administering the compounds of the present invention in combination with a therapeutically effective amount of another agent known to be useful to treat depression or anxiety.

The present invention also relates to the use of the compounds of the present invention in the manufacture of a medicament for the treatment, control or prevention of disorders, diseases, or conditions responsive to antagonism of SSTR5.

The present invention also relates to the use of the compounds of the present invention in the manufacture of a medicament for the treatment, control or prevention of type 2 diabetes, hyperglycemia, insulin resistance, obesity, lipid disorders, atherosclerosis, and metabolic syndrome.

The present invention also relates to the use of the compounds of the present invention in the manufacture of a medicament for the treatment, control or prevention of depression, and anxiety.

The present invention also relates to the use of the compounds of the present invention in the manufacture of a medicament for the suppression of GLP-1 secretion in a subject in need thereof.

The present invention also relates to the use of the compounds of the present invention in the manufacture of a medicament that also includes a therapeutically effective amount of another agent for the treatment of diabetes.

### DETAILED DESCRIPTION OF THE INVENTION

The present invention is concerned with substituted spirocyclic amines useful as antagonists of SSTR5. Compounds of the present invention are described by structural formula I:

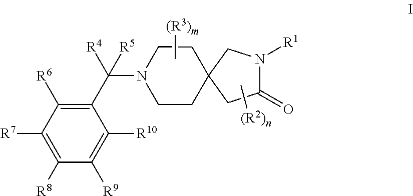

and pharmaceutically acceptable salts thereof, wherein
R¹ is selected from the group consisting of
(1) hydrogen,
(2) —C₁₋₁₀alkyl,
(3) —(CH₂)ₛOH,
(4) —(CH₂)ₛOR^{e},
(5) —(CH₂)ₛNR^{c}R^{d},
(6) —(CH₂)ₛOC₁₋₁₀alkyl,
(7) —(CH₂)ᵣCO₂H,
(8) —(CH₂)ᵣCO₂R^{e},
(9) —(CH₂)ᵣCONR^{c}R^{d},
(10) —(CH₂)ᵣCOR^{e},
(11) —S(O)_{q}C₁₋₁₀alkyl,
(12) —S(O)_{q}(CH₂)ₚaryl,
(13) —S(O)_{q}(CH₂)ₚcycloalkyl,
(14) —S(O)_{q}(CH₂)ₚcycloheteroalkyl,
(15) —S(O)_{q}(CH₂)ₚheteroaryl,
(16) —(CH₂)ₚC₃₋₁₀ cycloalkyl,
(17) —(CH₂)ₚC₂₋₁₀ cycloheteroalkyl,
(18) —(CH₂)ₚaryl, and
(19) —(CH₂)ₚheteroaryl,
   wherein CH₂, alkyl, cycloalkyl, cycloheteroalkyl, aryl and heteroaryl are unsubstituted or substituted with one, two or three substituents independently selected from R^{a};
   R² is selected from the group consisting of:
(1) hydrogen,
(2) C₁₋₆alkyl, and
(3) —OC₁₋₆alkyl;
   R³ is selected from the group consisting of:
(1) hydrogen, and
(2) C₁₋₆alkyl;
   R⁴ is selected from the group consisting of:
(1) hydrogen, and
(2) —C₁₋₆ alkyl;
   R⁵ is selected from the group consisting of
(1) hydrogen, and
(2) —C₁₋₆ alkyl,
   or R⁴ and R⁵ together with the atom to which they are attached form a cycloalkyl ring with 3 to 7 carbon atoms;
   R⁶ is selected from the group consisting of:
(1) hydrogen,
(2) halogen,
(3) —C₁₋₁₀ alkyl,
(4) —OC₁₋₁₀ alkyl,
(5) aryl, and
(6) heteroaryl;
   R⁷ is selected from the group consisting of:
(1) hydrogen,
(2) —C₁₋₁₀
(3) —C₃₋₁₀cycloalkyl,
(4) —OH,
(5) —O—C₁₋₁₀alkyl,
(6) —O—C₃₋₁₀cycloalkyl,
(7) —O—C₂₋₁₀cycloheteroalkyl,
(8) —O-aryl,
(9) —O-heteroaryl,
(10) —NR^{c}S(O)ₜR^{e},
(11) halogen,
(12) —NR^{c}R^{d},
(13) —CN,
(14) —NR^{c}C(O)R^{e},
(15) —OCF₃,
(16) —OCHF₂,
(17) C₂₋₁₀cycloheteroalkyl,
(18) aryl, and
(19) heteroaryl,
   wherein alkyl, cycloalkyl, cycloheteroalkyl, aryl and heteroaryl are unsubstituted or substituted with 1, 2 or 3 halogens;
   R⁸ is selected from the group consisting of:
(1) —OC₁₋₆alkyl,
(2) —NR^{c}S(O)ᵤR^{e},
(3) halogen,
(4) —S(O)ᵤR^{e},
(5) —S(O)ᵤNR^{c}R^{d},
(6) —NR^{c}R^{d},
(7) —CN,
(8) —C(O)NR^{c}R^{d},
(9) —NR^{c}C(O)R^{e},
(10) —NR^{c}C(O)OR^{e},
(11) —NR^{c}C(O)NR^{c}R^{d},
(12) —OCF₃,
(13) —OCHF₂,
(14) C₃₋₁₀cycloheteroalkyl,
(15) C₁₋₁₀alkyl,
(16) C₃₋₆cycloalkyl,
(17) aryl, and
(18) heteroaryl,
   wherein alkyl, cycloalkyl, cycloheteroalkyl, aryl and heteroaryl are unsubstituted or substituted with one, two or three substituents independently selected from R^{b};
   each R⁹ is selected from the group consisting of:
(1) hydrogen,
(2) —C₁₋₁₀alkyl,
(3) —C₃₋₁₀cycloalkyl,
(4) —OH,
(5) —O—C₁₋₁₀alkyl,
(6) —O—C₃₋₁₀cycloalkyl,
(7) —O—C₂₋₁₀cycloheteroalkyl,
(8) —O-aryl,
(9) —O-heteroaryl,
(10) —NR^{c}S(O)ₜR^{e},
(11) halogen,
(12) —NR^{c}R^{d},
(13) —CN,
(14) —NR^{c}C(O)R^{e},
(15) —OCF₃,
(16) —OCHF₂,
(17) C₂₋₁₀cycloheteroalkyl,
(18) aryl, and
(19) heteroaryl,
   wherein alkyl, cycloalkyl, cycloheteroalkyl, aryl and heteroaryl are unsubstituted or substituted with 1, 2 or 3 halogens;
   R¹⁰ is selected from the group consisting of:
(1) hydrogen,
(2) halogen,
(3) —C₁₋₁₀ alkyl, and
(4) —OC₁₋₁₀ alkyl;
   each R^{a} is independently selected from the group consisting of:
(1) —C₁₋₆alkyl,
(2) —CF₃,
(3) —OH,
(4) —OC₁₋₆alkyl,
(5) —OCF₃,
(6) —OCHF₂,
(7) —OCH₂F,
(8) halogen,
(9) —S(O)ᵥR^{e},
(10) —S(O)ᵥNR^{c}R^{d},
(11) —NR^{c}S(O)ᵥR^{e},
(12) —NO₂,
(13) —NR^{c}R^{d},
(14) —C(O)R^{e},
(15) —CO₂H,
(16) —CO₂R^{e},
(17) —OC(O)R^{e},
(18) —CN,
(19) —C(O)NR^{c}R^{d},
(20) —NR^{c}C(O)R^{e},
(21) —NR^{c}C(O)OR^{e},
(22) —NR^{c}C(O)NR^{c}R^{d},
(23) C₃₋₁₀ cycloalkyl,
(24) C₂₋₁₀ cycloheteroalkyl,
(25) aryl, and
(26) heteroaryl,
   wherein alkyl, cycloalkyl, cycloheteroalkyl, aryl and heteroaryl are unsubstituted or substituted with 1 or 2 substituents selected from oxo, C₁₋₆alkyl, —CO₂H, —NH₂, NH(C₁₋₆alkyl), and NH(C₁₋₆alkyl)₂;
   each R^{b} is independently selected from the group consisting of:
(1) —CN,
(2) halogen,
(3) —CF₃,
(4) —OCF₃,
(5) —C₁₋₆alkyl,
(6) —OC₁₋₆alkyl,
(7) aryl, and
(8) heteroaryl;
   each R^{c} is independently selected from the group consisting of:
(1) hydrogen, and
(2) C₁₋₆alkyl;
   each R^{d} is independently selected from the group consisting of:
(1) hydrogen, and
(2) C₁₋₆alkyl;
   each R^{e} is independently selected from the group consisting of:
(1) C₁₋₆ alkyl,
(2) C₃₋₁₀cycloalkyl,
(3) C₂₋₁₀cycloheteroalkyl,
(4) aryl, and
(5) heteroaryl;
   m is 0, 1, 2, 3 or 4;
   n is 0, 1 or 2;
   p is 0, 1, 2, 3, 4 or 5;
   q, t, u and v are 1 or 2;
   r is 1, 2, 3, 4 or 5; and
   s is 2, 3 or 4.

The invention has numerous embodiments, which are summarized below. The invention includes compounds of Formula I, which includes compounds of formula Ia, Ib, Ic, and Id. The invention also includes pharmaceutically acceptable salts of the compounds and pharmaceutical compositions comprising the compounds and a pharmaceutically acceptable carrier. The compounds are useful for the treatment of Type 2 diabetes, hyperglycemia, obesity, and lipid disorders that are associated with Type 2 diabetes.

In one embodiment of the present invention, R¹ is selected from the group consisting of: hydrogen, —C₁₋₁₀alkyl, —(CH₂)ₛOR^{e}, —(CH₂)ₛNR^{c}R^{d}, —(CH₂)ₛOC₁₋₁₀alkyl, —(CH₂)ᵣCO₂H, —(CH₂)ᵣCO₂R^{e}, —(CH₂)ᵣCONR^{c}R^{d}, —(CH₂)ᵣCOR^{e}, —S(O)C₁₋₁₀alkyl, —S(O)_{q}(CH₂)ₚaryl, —S(O)_{q}(CH₂)ₚcycloalkyl, —S(O)_{q}(CH₂)ₚcycloheteroalkyl, —S(O)_{q}(CH₂)ₚheteroaryl, —(CH₂)ₚC₃₋₁₀cycloalkyl, —(CH₂)ₚC₂₋₁₀ cycloheteroalkyl, —(CH₂)ₚaryl, and —(CH₂)ₚheteroaryl, wherein CH₂, alkyl, cycloalkyl, cycloheteroalkyl, aryl and heteroaryl are unsubstituted or substituted with one, two or three substituents independently selected from R^{a}.

In a class of this embodiment of the present invention, R¹ is selected from the group consisting of hydrogen, —C₁₋₁₀alkyl, —(CH₂)ₛOR^{e}, —(CH₂)ₛNR^{c}R^{d}, —(CH₂)ₛOC₁₋₁₀alkyl, —(CH₂)ᵣCO₂H, —(CH₂)ᵣCO₂R^{e}, —(CH₂)ᵣCONR^{c}R^{d}, —(CH₂)ᵣCOR^{e}, —S(O)_{q}C₁₋₁₀alkyl, —S(O)_{q}(CH₂)ₚaryl, —S(O)_{q}(CH₂)ₚcycloalkyl, —S(O)_{q}(CH₂)ₚcycloheteroalkyl, S(O)_{q}(CH₂)ₚheteroaryl, —(CH₂)ₚC₃₋₁₀ cycloalkyl, —(CH₂)ₚC₂₋₁₀ cycloheteroalkyl, —(CH₂)ₚaryl, and —(CH₂)ₚheteroaryl, wherein CH₂, alkyl, cycloalkyl and eycloheteroalkyl are unsubstituted or substituted with one, two or three substituents independently selected from R^{a}, and wherein aryl and heteroaryl are substituted with one, two or three substituents independently selected from R^{a}.

In another class of this embodiment of the present invention, R¹ is selected from the group consisting of: —C₁₋₁₀alkyl, —(CH₂)ₛOR^{e}, —(CH₂)ₛNR^{c}R^{d}, —(CH₂)ₛOC₁₋₁₀alkyl, —(CH₂)ᵣCO₂H, —(CH₂)ᵣCO₂R^{e}, —(CH₂)ᵣCONR^{c}R^{d}, —(CH₂)ᵣCOR^{e}, —S(O)_{q}C₁₋₁₀alkyl, —S(O)_{q}(CH₂)ₚaryl, —S(O)_{q}(CH₂)ₚcycloalkyl, —S(O)_{q}(CH₂)ₚcycloheteroalkyl, —S(O)_{q}(CH₂)ₚhoteroaryl, —(CH₂)ₚC₃₋₁₀ cycloalkyl, —(CH₂)ₚC₂₋₁₀ cycloheteroalkyl, —(CH₂)ₚaryl, and —(CH₂)ₚheteroaryl, wherein CH₂, alkyl, cycloalkyl and cycloheteroalkyl are unsubstituted or substituted with one, two or three substituents independently selected from R^{a}, and wherein aryl and heteroaryl are substituted with one, two or three substituents independently selected from R^{a}.

In another class of this embodiment, R¹ is selected from the group consisting of: hydrogen, —C₁₋₁₀alkyl, —(CH₂)ₛOH, —(CH₂)ₛNR^{c}R^{d}, —(CH₂)ₛOC₁₋₁₀alkyl, —(CH₂)ᵣCO₂H, —(CH₂)ᵣCO₂C₁₋₁₀alkyl, —(CH₂)ᵣCONR^{c}R^{d}, —(CH₂)ᵣCO-cycloheteroalkyl, —S(O)_{q}C₁₋₁₀alkyl, —S(O)_{q}(CH₂)ₚaryl, —S(O)_{q}(CH₂)ₚcycloalkyl, —S(O)_{q}(CH₂)ₚcycloheteroalkyl, S(O)_{q}(CH₂)ₚheteroaryl, —(CH₂)ₚC₃₋₁₀ cycloalkyl, —(CH₂)ₚC₂₋₁₀ cycloheteroalkyl, —(CH₂)ₚaryl, and —(CH₂)ₚheteroaryl, wherein CH₂, alkyl, cycloalkyl, cycloheteroalkyl, aryl and heteroaryl are unsubstituted or substituted with one, two or three substituents independently selected from R^{a}.

In a class of this embodiment, R¹ is selected from the group consisting of: —C₁₋₁₀alkyl, —(CH₂)ₛOH, —(CH₂)ₛNR^{c}R^{d}, —(CH₂)ₛOC₁₋₁₀alkyl, —(CH₂)ᵣCO₂H, —(CH₂)ᵣCO₂C₁₋₁₀alkyl, (CH₂)ᵣCONR^{c}R^{d}, —(CH₂)ᵣCO-cycloheteroalkyl, —S(O)_{q}C₁₋₁₀alkyl, —S(O)_{q}(CH₂)ₚaryl, —S(O)_{q}(CH₂)ₚcycloalkyl, —S(O)_{q}(CH₂)ₚcycloheteroalkyl, —S(O)_{q}(CH₂)ₚheteroaryl, —(CH₂)ₚC₃₋₁₀cycloalkyl, —(CH₂)ₚC₂₋₁₀ cycloheteroalkyl, —(CH₂)ₚaryl, and —(CH₂)ₚheteroaryl, wherein CH₂, alkyl, cycloalkyl and cycloheteroalkyl are unsubstituted or substituted with one, two or three substituents independently selected from R^{a}, and wherein aryl and heteroaryl are substituted with one, two or three substituents independently selected from R^{a}.

In another class of this embodiment, R¹ is selected from the group consisting of: hydrogen, —C₁₋₁₀alkyl, —(CH₂)ₛOH, —(CH₂)ₛN^{c}R^{d}, —(CH₂)ₛOC₁₋₁₀alkyl, —(CH₂)ᵣCO₂H, —(CH₂)ᵣCO₂C₁₋₁₀alkyl, —(CH₂)ᵣCONR^{c}R^{d}, —(CH₂)ᵣCO-cycloheteroalkyl, —S(O)_{q}C₁₋₁₀alkyl, —S(O)_{q}(CH₂)ₚaryl, —S(O)_{q}(CH₂)ₚcycloalkyl, —S(O)₂cycloheteroalkyl, —S(O)₂heteroaryl, (CH₂)ₚC₃₋₁₀ cycloalkyl, —(CH₂)ₚC₂₋₁₀ cycloheteroalkyl, —(CH₂)ₚaryl, and —(CH₂)ₚheteroaryl, wherein CH₂, alkyl, cycloalkyl, cycloheteroalkyl, aryl and heteroaryl are unsubstituted or substituted with one, two or three substituents independently selected from R.

In another class of this embodiment, R¹ is selected from the group consisting of: —C₁₋₁₀alkyl, —(CH₂)ₛOH, —(CH₂)ₛNR^{c}R^{d}, —(CH₂)ₛOC₁₋₁₀alkyl, —(CH₂)ᵣCO₂H, —(CH₂)ᵣCO₂C₁₋₁₀alkyl, —(CH₂)ᵣCONR^{c}R^{d}, —(CH₂)ᵣCO-cycloheteroalkyl, —S(O)_{q}C₁₋₁₀alkyl, —S(O)_{q}(CH₂)ₚaryl, —S(O)_{q}(CH₂)ₚcycloalkyl, —S(O)₂cycloheteroalkyl, —S(O)₂heteroaryl, —(CH₂)ₚC₃₋₁₀ cycloalkyl, —(CH₂)ₚC₂₋₁₀ cycloheteroalkyl, —(CH₂)ₚaryl, and —(CH₂)ₚheteroaryl, wherein CH₂, alkyl, cycloalkyl, and cycloheteroalkyl are unsubstituted or substituted with one, two or three substituents independently selected from R^{a}, and wherein aryl and heteroaryl are substituted with one, two or three substituents independently selected from R^{a}.

In another class of this embodiment, R¹ is selected from the group consisting of hydrogen, —(CH₂)ₛOH, —(CH₂)ᵣCO₂H, —(CH₂)ᵣCO₂C₁₋₁₀alkyl, —(CH₂)ᵣCONR^{c}R^{d}, S(O)_{q}(CH₂)ₚaryl, —(CH₂)ₚaryl, and —(CH₂)ₚheteroaryl, wherein CH₂, alkyl, aryl and heteroaryl are unsubstituted or substituted with one, two or three substituents independently selected from R^{a}.

In another class of this embodiment, R¹ is selected from the group consisting of: hydrogen, —(CH₂)ₛOH, —(CH₂)ᵣCO₂H, —(CH₂)ᵣCO₂C₁₋₁₀alkyl, —(CH₂)ᵣCONR^{c}R^{d}, S(O)_{q}(CH₂)ₚaryl, —(CH₂)ₚaryl, and —(CH₂)ₚheteroaryl, wherein CH₂ and alkyl are unsubstituted or substituted with one, two or three substituents independently selected from R^{a}, and wherein aryl and heteroaryl are substituted with one, two or three substituents independently selected from R^{a}.

In another class of this embodiment, R¹ is selected from the group consisting of: —(CH₂)ₛOH, —(CH₂)ᵣCO₂H, —(CH₂)ᵣCO₂C₁₋₁₀alkyl, —(CH₂)ᵣCONR^{c}R^{d}, —S(O)_{q}(CH₂)ₚaryl, —(CH₂)ₚaryl, and —(CH₂)ₚheteroaryl, wherein CH₂ and alkyl are unsubstituted or substituted with one, two or three substituents independently selected from R^{a}, and wherein aryl and heteroaryl are substituted with one, two or three substituents independently selected from R^{a}.

In another class of this embodiment, R¹ is selected from the group consisting of: hydrogen, —(CH₂)₂₋₃OH, —(CH₂)₁₋₄CO₂H, —(CH₂)₁₋₄CO₂C₁₋₁₀alkyl, —(CH₂)₁₋₃CONH₂, —S(O)₂aryl, —(CH₂)₀₋₁aryl, and heteroaryl, wherein CH₂, alkyl, aryl and heteroaryl are unsubstituted or substituted with one or two substituents independently selected from R^{a}.

In another class of this embodiment, R¹ is selected from the group consisting of: —(CH₂)₂₋₃OH, —(CH₂)₁₋₄CO₂H, —(CH₂)₁₋₄CO₂C₁₋₁₀alkyl, —(CH₂)₁₋₃CONH₂, —S(O)₂aryl, —(CH₂)₀₋₁aryl, and heteroaryl, wherein CH₂ and alkyl are unsubstituted or substituted with one or two substituents independently selected from R^{a}, and wherein aryl and heteroaryl are substituted with one or two substituents independently selected from R^{a}.

In another class of this embodiment, R¹ is selected from the group consisting of: hydrogen, —(CH₂)₂₋₃OH, —(CH₂)₁₋₄CO₂H, —(CH₂)₁₋₄CO₂C₁₋₂alkyl, —(CH₂)₁₋₃CONH₂, —S(O)₂-phenyl, phenyl, —CH₂-phenyl, and heteroaryl, wherein CH₂, alkyl, phenyl and heteroaryl are unsubstituted or substituted with one, two or three substituents independently selected from. R^{a}.

In another class of this embodiment, R¹ is selected from the group consisting of hydrogen, —(CH₂)₂₋₃OH, —(CH₂)₁₋₄CO₂H, —(CH₂)₁₋₄ CO₂C₁₋₂alkyl, —(CH₂)₁₋₃CONH₂, S(O)₂phenyl, phenyl, —CH₂phenyl, and heteroaryl, wherein CH₂ and alkyl are unsubstituted or substituted with one, two or three substituents independently selected from R^{a}, and wherein phenyl and heteroaryl are substituted with one, two or three substituents independently selected from R^{a}.

In another class of this embodiment, R¹ is selected from the group consisting of: hydrogen, —(CH₂)₂₋₃OH, —(CH₂)₁₋₄CO₂H, —(CH₂)₁₋₄CO₂CH₃, —(CH₂)₁₋₄CO₂CH₂CH₃, —(CH₂)₁₋₃CONH₂, —S(O)₂phenyl, phenyl, —CH₂phenyl, pyridine, and pyrimidine, wherein CH₂, alkyl, phenyl, pyridine and pyrimidine are unsubstituted or substituted with one or two substituents independently selected from R^{a}.

In another class of this embodiment, R¹ is selected from the group consisting of: hydrogen, —(CH₂)₂₋₃OH, —(CH₂)₁₋₄CO₂H, —(CH₂)₁₋₄CO₂CH₃, —(CH₂)₁₋₄CO₂CH₂CH₃, —(CH₂)₁₋₃CONH₂, —S(O)₂phenyl, phenyl, —CH₂phenyl, pyridine, and pyrimidine, wherein CH₂ and alkyl are unsubstituted or substituted with one or two substituents independently selected from R^{a}, and wherein phenyl, pyridine and pyrimidine are substituted with one or two substituents independently selected from R^{a}.

In another class of this embodiment, R¹ is selected from the group consisting of: —(CH₂)₂₋₃OH, —(CH₂)₁₋₄CO₂H, —(CH₂)₁₋₄CO₂CH₃, —(CH₂)₁₋₄CO₂CH₂CH₃, —(CH₂)₁₋₃CONH₂, —S(O)₂phenyl, phenyl, —CH₂phenyl, pyridine, and pyrimidine, wherein CH₂ and alkyl are unsubstituted or substituted with one or two substituents independently selected from R^{a}, and wherein phenyl, pyridine and pyrimidine are substituted with one or two substituents independently selected from R^{a}.

In another class of this embodiment, R¹ is selected from the group consisting of: hydrogen, —(CH₂)₂₋₃OH, —(CH₂)₁₋₄CO₂H, —(CH₂)₁₋₄CO₂CH₃, —(CH₂)₂CO₂CH₂CH₃, —(CH₂)₁₋₃CONH₂, —S(O)₂phenyl, phenyl, —CH₂phenyl, pyridine, and pyrimidine, wherein CH₂, alkyl, phenyl, pyridine and pyrimidine are unsubstituted or substituted with one or two substituents independently selected from R^{a}.

In another class of this embodiment, R¹ is selected from the group consisting of: hydrogen, —(CH₂)₂₋₃OH, —(CH₂)₁₋₄CO₂H, —(CH₂)₁₋₄CO₂CH₃, —(CH₂)₂CO₂CH₂CH₃, —(CH₂)₁₋₃CONH₂, —S(O)₂phenyl, phenyl, —CH₂phenyl, pyridine, and pyrimidine, wherein CH₂ and alkyl are unsubstituted or substituted with one or two substituents independently selected from R^{a}, and wherein phenyl, pyridine and pyrimidine are substituted with one or two substituents independently selected from R^{a}.

In another class of this embodiment, R¹ is selected from the group consisting of: —(CH₂)₂₋₃OH, —(CH₂)₁₋₄CO₂H, —(CH₂)₁₋₄CO₂CH₃, —(CH₂)₂CO₂CH₂CH₃, —(CH₂)₁₋₃CONH₂, —S(O)₂phenyl, phenyl, —CH₂phenyl, pyridine, and pyrimidine, wherein CH₂ and alkyl are unsubstituted or substituted with one or two substituents independently selected from R^{a}, and wherein phenyl, pyridine and pyrimidine are substituted with one or two substituents independently selected from R^{a}.

In another class of this embodiment, R¹ is selected from the group consisting of: hydrogen, aryl and heteroaryl, wherein aryl and heteroaryl are unsubstituted or substituted with one substituent independently selected from R^{a}. In a subclass of this class, R¹ is selected from the group consisting of hydrogen, phenyl and pyridine, wherein phenyl and pyridine are unsubstituted or substituted with one substituent independently selected from R^{a}.

In another class of this embodiment, R¹ is selected from the group consisting of: hydrogen, aryl and heteroaryl, wherein aryl and heteroaryl are substituted with one substituent independently selected from R^{a}. In a subclass of this class, R¹ is selected from the group consisting of: hydrogen, phenyl and pyridine, wherein phenyl and pyridine are substituted with one substituent independently selected from R^{a}. In another subclass of this class, R¹ is selected from the group consisting of: hydrogen, phenyl and pyridine, wherein phenyl and pyridine are substituted with one substituent independently selected from: —CO₂H and tetrazole.

In another class of this embodiment, R¹ is selected from the group consisting of: aryl and heteroaryl, wherein aryl and heteroaryl are substituted with one substituent independently selected from R^{a}. In a subclass of this class, R¹ is selected from the group consisting of: phenyl and pyridine, wherein phenyl and pyridine are substituted with one substituent independently selected from R^{a}. In another subclass of this class, R¹ is selected from the group consisting of: phenyl and pyridine, wherein phenyl and pyridine are substituted with one substituent independently selected from: —CO₂H and tetrazole. In another subclass of this class, R¹ is selected from the group consisting of: phenyl and pyridine, wherein phenyl and pyridine are substituted with one —CO₂H substituent.

In another class of this embodiment, R¹ is selected from the group consisting of: hydrogen, and aryl, wherein aryl is unsubstituted or substituted with one substituent independently selected from R^{a}. In a subclass of this class, R¹ is selected from the group consisting of hydrogen, and phenyl, wherein phenyl is unsubstituted or substituted with one substituent independently selected from R^{a}. In another subclass of this class, R¹ is aryl, wherein aryl is substituted with one substituent independently selected from R^{a}. In another subclass of this class, R¹ is phenyl, wherein phenyl is substituted with one substituent independently selected from R^{a}. In another subclass of this class, R¹ is phenyl, wherein phenyl is substituted with one substituent independently selected from: —CO₂H and tetrazole. In another subclass of this class, R¹ is phenyl, wherein phenyl is substituted with one —CO₂H substituent.

In another class of this embodiment, R¹ is heteroaryl, wherein heteroaryl is substituted with one substituent independently selected from R^{a}. In a subclass of this class, R¹ is pyridine, wherein pyridine is substituted with one substituent independently selected from R^{a}. In another subclass of this class, R¹ is pyridine, wherein pyridine is substituted with one substituent independently selected from: —CO₂H and tetrazole. In another subclass of this class, R¹ is pyridine, wherein pyridine is substituted with one —CO₂H substituent.

In another embodiment of the present invention, R² is selected from the group consisting of: hydrogen, —C₁₋₆alkyl, and —OC₁₋₆alkyl. In a class of this embodiment, R² is hydrogen.

In another embodiment of the present invention, R³ is selected from the group consisting of: hydrogen, and C₁₋₆alkyl. In a class of this embodiment, R³ is hydrogen.

In another embodiment of the present invention, R⁴ is selected from the group consisting of: hydrogen, and —C₁₋₆ alkyl. In a class of this embodiment, R⁴ is hydrogen.

In another embodiment of the present invention, R⁵ is selected from the group consisting of: hydrogen, and —C₁₋₆ alkyl, or R⁴ and R⁵ together with the atom to which they are attached form a cycloalkyl ring with 3 to 7 carbon atoms. In a class of this embodiment, R⁵ is selected from the group consisting of: hydrogen, and —C₁₋₆ alkyl. In another class of this embodiment, R⁵ is hydrogen.

In another embodiment of the present invention, R⁶ is selected from the group consisting of: hydrogen, halogen, —C₁₋₁₀ alkyl, —OC₁₋₁₀ alkyl, aryl, and heteroaryl. In a class of this embodiment, R⁶ is selected from the group consisting of: hydrogen, halogen, —C₁₋₁₀ alkyl, —OC₁₋₁₀ alkyl, phenyl, and heteroaryl. In another class of this embodiment, R⁶ is selected from the group consisting of: hydrogen, and halogen. In another class of this embodiment, R⁶ is hydrogen. In another class of this embodiment, R⁶ is halogen. In a subclass of this class, R⁶ is Br.

In another embodiment of the present invention, each R⁷ is selected from the group consisting of: hydrogen, —C₁₋₁₀alkyl, —C₃₋₁₀cycloalkyl, —O—C₁₋₁₀alkyl, —O—C₃₋₁₀cycloalkyl, —O—C₂₋₁₀cycloheteroalkyl, —O-aryl, —O-heteroaryl, —NR^{c}S(O)ₜR^{e}, halogen, —NR^{c}R^{d}, —CN, —NR^{c}C(O)R^{e}, —OCF₃, —OCHF₂, —C₂₋₁₀cycloheteroalkyl, aryl, and heteroaryl, wherein alkyl, cycloalkyl, cycloheteroalkyl, aryl and heteroaryl are unsubstituted or substituted with 1, 2 or 3 halogens. In a class of this embodiment, R⁷ is selected from the group consisting of: —O—C₁₋₁₀alkyl, and —O—C₃₋₁₀cycloalkyl, wherein alkyl and cycloalkyl are unsubstituted or substituted with 1, 2 or 3 halogens. In a subclass of this class of this embodiment, R⁷ is selected from the group consisting of: —O—CH₂CH₃, and —O-cyclopropyl, wherein alkyl and cycloalkyl are unsubstituted or substituted with 1, 2 or 3 halogens. In another class of this embodiment, R⁷ is selected from the group consisting of: —O—C₁₋₁₀alkyl, and —O—C₃₋₁₀cycloalkyl. In a subclass of this class of this embodiment, R⁷ is selected from the group consisting of: —O—CH₂CH₃, and —O-cyclopropyl. In another class of this embodiment, R⁷ is —O—C₁₋₁₀alkyl, wherein alkyl is unsubstituted or substituted with 1, 2 or 3 halogens. In a subclass of this class of this embodiment, R⁷ is —O—C₁₋₁₀alkyl. In another subclass of this class of this embodiment, R⁷ is —O—CH₂CH₃. In another class of this embodiment, R⁷ is selected from the group consisting of: —O—C₃₋₁₀cycloalkyl, wherein cycloalkyl is unsubstituted or substituted with 1, 2 or 3 halogens. In another class of this embodiment, R⁷ is selected from the group consisting of: —O—C₃₋₁₀cycloalkyl. In a subclass of this class, R⁷ is —O-cyclopropyl.

In another embodiment of the present invention, R⁸ is selected from the group consisting of: —OC₁₋₆alkyl, —NR^{c}S(O)ᵤR^{e}, halogen, —S(O)ᵤR^{e}, —S(O)ᵤNR^{c}R^{d}, —NR^{c}R^{d}, —CN, —C(O)NR^{c}R^{d}, —NR^{c}C(O)R^{e}, —NR^{c}C(O)OR^{e}, —NR^{c}C(O)NR^{c}R^{d}, —OCF₃, —OCHF₂, —C₃₋₁₀cycloheteroalkyl, —C₃₋₆cycloalkyl, aryl, and heteroaryl, wherein alkyl, cycloalkyl, cycloheteroalkyl, aryl and heteroaryl are unsubstituted or substituted with one, two or three substituents independently selected from R^{b}.

In a class of this embodiment, R⁸ is selected from the group consisting of: —OC₁₋₆alkyl, halogen, aryl, and heteroaryl, wherein alkyl, aryl and heteroaryl are unsubstituted or substituted with one, two or three substituents independently selected from R^{b}. In a subclass of this class, R⁸ is selected from the group consisting of: —OC₁₋₆alkyl, halogen, phenyl, and pyridine, wherein alkyl, phenyl and pyridine are unsubstituted or substituted with one or two substituents independently selected from R^{b}. In another subclass of this class, R⁸ is selected from the group consisting of: —OC₁₋₆alkyl, Br, F, phenyl, and pyridine, wherein alkyl, phenyl and pyridine are unsubstituted or substituted with one or two substituents independently selected from R^{b}.

In another class of this embodiment, R⁸ is selected from the group consisting of: halogen, aryl, and heteroaryl, wherein aryl and heteroaryl are unsubstituted or substituted with one, two or three substituents independently selected from R^{b}. In a subclass of this class, R⁸ is selected from the group consisting of: halogen, phenyl, and heteroaryl, wherein phenyl and heteroaryl are unsubstituted or substituted with one, two or three substituents independently selected from R^{b}. In another subclass of this class, R⁸ is selected from the group consisting of halogen, phenyl, and pyridine, wherein phenyl and pyridine are unsubstituted or substituted with one, two or three substituents independently selected from R^{b}. In another subclass of this class, R⁸ is selected from the group consisting of: Br, F, phenyl, and pyridine, wherein phenyl and pyridine are unsubstituted or substituted with one, two or three substituents independently selected from R^{b}.

In another embodiment of the present invention, R⁸ is selected from the group consisting of: aryl, and heteroaryl, wherein aryl and heteroaryl are unsubstituted or substituted with one or two substituents independently selected from R^{b}. In a class of this embodiment, R⁸ is selected from the group consisting of: phenyl, and heteroaryl, wherein phenyl and heteroaryl are unsubstituted or substituted with one or two substituents independently selected from R^{b}. In another class of this embodiment, R⁸ is selected from the group consisting of: phenyl, and pyridine, wherein phenyl and pyridine are unsubstituted or substituted with one or two substituents independently selected from R^{b}.

In another embodiment of the present invention, R⁹ is selected from the group consisting of: hydrogen, —C₁₋₁₀alkyl, —C₃₋₁₀cycloalkyl, —OH, —O—C₁₋₁₀alkyl, —O—C₃₋₁₀cycloalkyl, —O—C₂₋₁₀cycloheteroalkyl, —O-aryl, —O-heteroaryl, —NR^{e}S(O)ₜR^{e}, halogen, —NR^{c}R^{d}, —CN, —NR^{c}C(O)R^{e}, —OCF₃, —OCHF₂, —C₂₋₁₀cycloheteroalkyl, aryl, and heteroaryl, wherein alkyl, cycloalkyl, cycloheteroalkyl, aryl and heteroaryl are unsubstituted or substituted with 1, 2 or 3 halogens. In a class of this embodiment, R⁹ is selected from the group consisting of: —O—C₁₋₁₀alkyl, and —O—C₃₋₁₀cycloalkyl, wherein alkyl and cycloalkyl are unsubstituted or substituted with 1, 2 or 3 halogens. In a subclass of this class of this embodiment, R⁹ is selected from the group consisting of: —O—CH₂CH₃, and —O-cyclopropyl, wherein alkyl and cycloalkyl are unsubstituted or substituted with 1, 2 or 3 halogens. In another class of this embodiment, R⁹ is selected from the group consisting of: —O—C₁₋₁₀alkyl, and —O—C₃₋₁₀cycloalkyl. In a subclass of this class of this embodiment, R⁹ is selected from the group consisting of: —O—CH₂CH₃, and —O-cyclopropyl. In another class of this embodiment, R⁹ is —O—C₁₋₁₀alkyl, wherein alkyl is unsubstituted or substituted with 1, 2 or 3 halogens. In a subclass of this class of this embodiment, R⁹ is —O—C₁₋₁₀alkyl. In another subclass of this class of this embodiment, R⁹ is —O—CH₂CH₃. In another class of this embodiment, R⁹ is selected from the group consisting of: —O—C₃₋₁₀cycloalkyl, wherein cycloalkyl is unsubstituted or substituted with 1, 2 or 3 halogens. In another class of this embodiment, R⁹ is selected from the group consisting of: —O—C₃₋₁₀cycloalkyl. In a subclass of this class, R⁹ is —O-cyclopropyl.

In another embodiment of the present invention, R¹⁰ is selected from the group consisting of: hydrogen, halogen, —C₁₋₁₀ alkyl, and —OC₁₋₁₀ alkyl. In a class of this embodiment, R¹⁰ is selected from the group consisting of: hydrogen, and halogen. In another class of this embodiment, R¹⁰ is hydrogen. In another class of this embodiment, R¹⁰ is halogen. In a subclass of this class, R¹⁰ is Br.

In another embodiment of the present invention, each R^{a} is independently selected from the group consisting of: —C₁₋₆alkyl, —CF₃, —OH, —OC₁₋₆alkyl, —OCF₃, —OCHF₂, —OCH₂F, halogen, —S(O)ᵥR^{e}, —S(O)ᵥNR^{c}R^{d}, —NR^{c}S(O)ᵥR^{e}, —NO₂, —NR^{c}R^{d}, —C(O)R^{e}, —CO₂H, —CO₂R^{e}, OC(O)R^{e}, —CN, —C(O)NR^{c}R^{d}, —NR^{c}C(O)R^{e}, —NR^{c}C(O)OR^{e}, —NR^{c}C(O)NR^{c}R^{d}, —C₃₋₁₀ cycloalkyl, —C₂₋₁₀ cycloheteroalkyl, aryl, and heteroaryl, wherein alkyl, cycloalkyl, cycloheteroalkyl, aryl and heteroaryl are unsubstituted or substituted with 1 or 2 substituents selected from oxo, C₁₋₆alkyl, —CO₂H, —NH₂, NH(C₁₋₆alkyl), and NH(C₁₋₆alkyl)₂.

In a class of this embodiment, each R^{a} is independently selected from the group consisting of: —C₁₋₆alkyl, —CF₃, —OH, —OC₁₋₆alkyl, —OCF₃, —OCHF₂, —OCH₂F, halogen, —S(O)ᵥR^{e}, —S(O)ᵥNR^{c}R^{d}, —NR^{e}S(O)ᵥR^{e}, —NO₂, —NR^{c}R^{d}, —C(O)R^{e}, —CO₂H, —CO₂R^{e}, —OC(O)R^{e}, —CN, —C(O)NR^{c}R^{d}, —NR^{c}C(O)R^{e}, —NR^{c}C(O)OR^{e}, and —NR^{c}C(O)NR^{c}R^{d}, wherein alkyl is unsubstituted or substituted with 1 or 2 substituents selected from oxo, C₁₋₆alkyl, —CO₂H, —NH₂, NH(C₁₋₆alkyl), and NH(C₁₋₆alkyl)₂.

In another class of this embodiment, each R^{a} is independently selected from the group consisting of: —C₁₋₆alkyl, —CF₃, —OH, —OC₁₋₆alkyl, —OCF₃, —OCHF₂, —OCH₂F, halogen, —S(O)ᵥC₁₋₆alkyl, —S(O)ᵥNR^{c}R^{d}, —NR^{c}S(O)ᵥR^{e}, —NO₂, —NR^{c}R^{d}, —C(O)C₁₋₆alkyl, —CO₂H, CO₂C₁₋₆alkyl, —OC(O)C₁₋₆alkyl, —CN, —C(O)NR^{c}R^{d}, —NR^{c}C(O)R^{e}, —NR^{c}C(O)OR^{e}, —NR^{c}C(O)NR^{c}R^{d}, —C₃₋₁₀ cycloalkyl, —C₂₋₁₀ cycloheteroalkyl, aryl, and heteroaryl, wherein alkyl, cycloalkyl, cycloheteroalkyl, aryl and heteroaryl are unsubstituted or substituted with 1 or 2 substituents selected from oxo, C₁₋₆alkyl, —CO₂H, —NH₂, NH(C₁₋₆alkyl), and NH(C₁₋₆alkyl)₂,

In another class of this embodiment, each R^{a} is independently selected from the group consisting of: —C₁₋₆alkyl, —CF₃, —OH, —OC₁₋₆alkyl, —OCF₃, —OCHF₂, —OCH₂F, halogen, —S(O)ᵥC₁₋₆alkyl, —S(O)ᵥNR^{c}R^{d}, —NR^{e}S(O)ᵥR^{e}, —NO₂, —NRCR^{d}, —C(O)C₁₋₆alkyl, —CO₂H, —CO₂C₁₋₆alkyl, —OC(O)C₁₋₆alkyl, —CN, —C(O)NR^{c}R^{d}, —NR^{c}C(O)R^{e}, —NR^{c}C(O)OR^{e}, and —NR^{c}C(O)NR^{c}R^{d}, wherein alkyl is unsubstituted or substituted with 1 or 2 substituents selected from oxo, C₁₋₆alkyl, —CO₂H, —NH₂, NH(C₁₋₆alkyl), and NH(C₁₋₆alkyl)₂.

In another class of this embodiment, each R^{a} is independently selected from the group consisting of: —OH, —CN, —OC₁₋₆alkyl, halogen, —S(O)₂C₁₋₆alkyl, —CO₂H, —CO₂C₁₋₆alkyl, C(O)NR^{c}R^{d}, and heteroaryl, wherein alkyl and heteroaryl are unsubstituted or substituted with 1 or 2 substituents selected from oxo, C₁₋₆alkyl, —CO₂H, —NH₂, NH(C₁₋₆alkyl), and NH(C₁₋₆alkyl)₂. In another class of this embodiment, each R^{a} is independently selected from the group consisting of —OH, —CN, —OC₁₋₆alkyl, halogen, —S(O)₂C₁₋₆alkyl, —CO₂H, —CO₂C₁₋₆alkyl, C(O)NR^{c}R^{d}, and heteroaryl, wherein heteroaryl is unsubstituted or substituted with 1 or 2 substituents selected from oxo. In another class of this embodiment, each R^{a} is independently selected from the group consisting of: —OH, —CN, —OC₁₋₆alkyl, halogen, —SO₂CH₃, —CO₂H, —CO₂C₁₋₆alkyl, —C(O)NH₂, tetrazole, and oxo-dihydro-oxadiazole. In another class of this embodiment, each R^{a} is independently selected from the group consisting of: —OH, —CN, —OCH₃, F, —SO₂CH₃, —CO₂H, —CO₂CH₃, —C(O)NH₂, tetrazole, and oxo-dihydro-oxadiazole.

In another class of this embodiment, each R^{a} is independently selected from the group consisting of: —OH, —CN, —OC₁₋₆alkyl, halogen, —S(O)₂C₁₋₆alkyl, —CO₂H, —CO₂C₁₋₆alkyl, and —C(O)NR^{c}R^{d}, wherein alkyl is unsubstituted or substituted with 1 or 2 substituents selected from oxo, C₁₋₆alkyl, —CO₂H, —NH₂, NH(C₁₋₆alkyl), and NH(C₁₋₆alkyl)₂.

In another class of this embodiment, each R^{a} is independently selected from the group consisting of: —OH, —CN, —OC₁₋₆alkyl, halogen, —S(O)₂C₁₋₆alkyl, —CO₂H, —CO₂C₁₋₆alkyl, —C(O)NR^{c}R^{d}, and heteroaryl, wherein heteroaryl is unsubstituted or substituted with 1 or 2 substituents selected from oxo. In another class of this embodiment, each R^{a} is independently selected from the group consisting of —OH, —OC₁₋₆alkyl, halogen, —SO₂CH₃, —CO₂H, —CO₂C₁₋₆alkyl, —C(O)NH₂, tetrazole, and oxo-dihydro-oxadiazole. In another class of this embodiment, each R^{a} is independently selected from the group consisting of: —OH, —OCH₃, F, —SO₂CH₃, —CO₂H, —CO₂CH₃, —C(O)NH₂, tetrazole, and oxo-dihydro-oxadiazole.

In another class of this embodiment, each R^{a} is independently selected from the group consisting of: —OH, —OC₁₋₆alkyl, halogen, —SO₂CH₃, —CO₂H, —CO₂C₁₋₆alkyl, and —C(O)NH₂, In another class of this embodiment, each R^{a} is independently selected from the group consisting of: —OH, —OCH₃, F, —SO₂CH₃, —CO₂H, —CO₂CH₃, and —C(O)NH₂.

In another class of this embodiment, each R^{a} is independently selected from the group consisting of: —CO₂H, —C(O)NR^{c}R^{d}, and heteroaryl, wherein heteroaryl is unsubstituted or substituted with 1 or 2 substituents selected from oxo. In another subclass of this class of this embodiment, each R^{a} is independently selected from the group consisting of: —CO₂H, —C(O)NH₂, tetrazole, and oxo-dihydro-oxadiazole. In another subclass of this class of this embodiment, each R^{a} is independently selected from the group consisting of: —CO₂H, —C(O)NH₂, tetrazole, and 5-oxo-4,5-dihydro-1,2,4-oxadiazole.

In another class of this embodiment, each R^{a} is independently selected from the group consisting of: —CO₂H, tetrazole, and —C(O)NR^{c}R^{d}. In a subclass of this class, each R^{a} is independently selected from the group consisting of: —CO₂H, tetrazole, and —C(O)NH₂. In another subclass of this class, each R^{a} is independently selected from the group consisting of: —CO₂H, and tetrazole. In another subclass of this class, each R^{a} is —CO₂H. In another subclass of this class, each R^{a} is tetrazole.

In another class of this embodiment, each R^{a} is independently selected from the group consisting of: —CO₂H, and —C(O)NR^{c}R^{d}. In a subclass of this class of this embodiment, each R^{a} is —CO₂H. In another subclass of this class of this embodiment, each R^{a} is —C(O)NRCR^{d}. In another subclass of this class of this embodiment, each R^{a} is —C(O)N142.

In another embodiment of the present invention, each R^{b} is independently selected from the group consisting of —CN, halogen, —CF₃, —OCF₃, —C₁₋₆alkyl, —OC₁₋₆alkyl, —C(O)NR^{c}R^{d}, aryl, and heteroaryl. In a class of this embodiment, each R^{b} is independently selected from the group consisting of: —CN, halogen, —CF₃, —OCF₃, —C₁₋₆alkyl, —OC₁₋₆alkyl, and —C(O)NR^{c}R^{d}.

In another class of this embodiment, each R^{b} is independently selected from the group consisting of: —CN, halogen, —CF₃, —OCF₃, —C₁₋₆alkyl, —OC₁₋₆alkyl, and —C(O)NH₂. In another class of this embodiment, each R^{b} is independently selected from the group consisting of: —CN, F, Cl, —CF₃, —OCF₃, —C₁₋₆alkyl, —OC₁₋₆alkyl, and —C(O)NH₂.

In another embodiment of the present invention, each R^{b} is independently selected from the group consisting of: —CN, halogen, —CF₃, —OCF₃, —C₁₋₆alkyl, and —C(O)NR^{c}R^{d}. In a class of this embodiment, each R^{b} is independently selected from the group consisting of: —CN, halogen, —CF₃, —OCF₃, —C₁₋₆alkyl, and —C(O)NR^{c}R^{d}. In another class of this embodiment, each R^{b} is independently selected from the group consisting of: —CN, halogen, —CF₃, —OCF₃, —C₁₋₆alkyl, and —C(O)NH₂. In another class of this embodiment, each R^{b} is independently selected from the group consisting of: —CN, F, Cl, —CF₃, —OCF₃, —C₁₋₆alkyl, and —C(O)NH₂.

In a class of this embodiment, each R^{b} is independently selected from the group consisting of: halogen, —CF₃, and —OCF₃. In another class of this embodiment, each R^{b} is independently selected from the group consisting of: halogen. In a subclass of this class, each R^{b} is independently selected from the group consisting of: Cl and F. In another subclass of this class, each R^{b} is Cland F. In yet another subclass of this class, each R^{b} is F.

In another embodiment of the present invention, each R^{e} is independently selected from the group consisting of: hydrogen, and C₁₋₆alkyl. In a class of this embodiment, each R^{e} is hydrogen. In another class of this embodiment, each R^{e} is C₁₋₆alkyl.

In another embodiment of the present invention, each R^{d} is independently selected from the group consisting of: hydrogen, and C₁₋₆alkyl. In a class of this embodiment, each R^{d} is hydrogen. In another class of this embodiment, each R^{d} is C₁₋₆alkyl.

In another embodiment of the present invention, each R^{e} is independently selected from the group consisting of: C₁₋₆ alkyl, C₃₋₁₀cycloalkyl, C₂₋₁₀cycloheteroalkyl, aryl, and heteroaryl. In a class of this embodiment, each R^{e} is independently selected from the group consisting of: C₁₋₆ alkyl, and aryl. In another class of this embodiment, R^{e} is C₁₋₆ alkyl. In another class of this embodiment, R^{e} is aryl.

In another embodiment, R², R³, R⁴, R⁵, R⁶ and R¹⁰ are each hydrogen. In another embodiment, R², R³, R⁴, R⁵ and R¹⁰ are each hydrogen. In another embodiment, R², R³, R⁴, R⁵ and R⁶ are each hydrogen. In another embodiment, R², R³, R⁴ and R⁵ are each hydrogen. In another embodiment, R² and R³ are each hydrogen. In another embodiment, R⁴ and R⁵ are each hydrogen.

In another embodiment of the present invention, R⁷ and R⁹ are independently selected from the group consisting of: —O—C₁₋₁₀ alkyl, and —O—C₃₋₁₀cycloalkyl, wherein alkyl and cycloalkyl are unsubstituted or substituted with 1, 2 or 3 halogens. In a subclass of this class of this embodiment, R⁷ and R⁹ are independently selected from the group consisting of: —O—CH₂CH₃, and —O-cyclopropyl, wherein alkyl and cycloalkyl are unsubstituted or substituted with 1, 2 or 3 halogens. In another class of this embodiment, R⁷ and R⁹ are independently selected from the group consisting of: —O—C₁₋₁₀alkyl, and —O—C₃₋₁₀cycloalkyl. In a subclass of this class of this embodiment, R⁷ and R⁹ are independently selected from the group consisting of —O—CH₂CH₃, and —O-cyclopropyl. In another class of this embodiment, R⁷ and R⁹ are independently selected from the group consisting of —O—C₁₋₁₀alkyl, wherein alkyl is unsubstituted or substituted with 1, 2 or 3 halogens. In a subclass of this class of this embodiment, R⁷ and R⁹ are independently selected from the group consisting of —O—C₁₋₁₀alkyl. In another subclass of this class of this embodiment, R⁷ and R⁹ are —O—CH₂CH₃. In another class of this embodiment, R⁷ and R⁹ are independently selected from the group consisting of: —O—C₃₋₁₀cycloalkyl, wherein cycloalkyl is unsubstituted or substituted with 1, 2 or 3 halogens. In another class of this embodiment, R⁷ and R⁹ are independently selected from the group consisting of: —O—C₃₋₁₀cycloalkyl. In a subclass of this class, R⁷ and R⁹ are —O-cyclopropyl.

In another embodiment of the present invention, m is 0, 1, 2, 3 or 4. In a class of this embodiment, m is 0, 1, 2 or 3. In another class of this embodiment, m is 0, 1 or 2. In another class of this embodiment, m is 0 or 1. In another class of this embodiment, m is 0. In another class of this embodiment, m is 1. In another class of this embodiment, m is 2. In another class of this embodiment, m is 3. In another class of this embodiment, m is 4.

In another embodiment of the present invention, n is 0, 1 or 2. In a class of this embodiment, n is 0. In a class of this embodiment, n is 1. In another class of this embodiment, n is 2.

In another embodiment of the present invention, p is 0, 1, 2, 3, 4 or 5. In a class of this embodiment, p is 0, 1, 2, 3 or 4. In another class of this embodiment, p is 0, 1, 2 or 3. In another class of this embodiment, p is 1, 2 or 3. In another class of this embodiment, p is 0 or 1. In another class of this embodiment, p is 0. In another class of this embodiment, p is I. In another class of this embodiment, p is 2. In another class of this embodiment, p is 3. In another class of this embodiment, p is 4. In another class of this embodiment, p is 5.

In another embodiment of the present invention, q is 1 or 2. In a class of this embodiment, q is 1. In another class of this embodiment, q is 2.

In another embodiment of the present invention, r is 1, 2, 3, 4 or 5. In a class of this embodiment, r is 1, 2, 3 or 4. In another class of this embodiment, r is 1, 2 or 3. In a class of this embodiment, r is 1 or 2. In another class of this embodiment, r is 1 or 3. In another class of this embodiment, r is 2 or 3. In another class of this embodiment, r is 1. In another class of this embodiment, r is 2. In another class of this embodiment, r is 3. In another class of this embodiment, r is 4. In another class of this embodiment, r is 5.

In another embodiment of the present invention, s is 2, 3 or 4. In a class of this embodiment, s is 2 or 3. In another class of this embodiment, s is 2 or 4. In another class of this embodiment, s is 2. In another class of this embodiment, s is 3. In another class of this embodiment, s is 4.

In another embodiment of the present invention, t is 1 or 2. In a class of this embodiment, t is 1. In another class of this embodiment, t is 2, In another embodiment of the present invention, u is 1 or 2. In a class of this embodiment, u is 1. In another class of this embodiment, u is 2.

In another embodiment of the present invention, v is 1 or 2. In a class of this embodiment, v is 1. In another class of this embodiment, v is 2.

In another embodiment of the present invention, are provided compounds of formula I wherein: R¹ is selected from the group consisting of: hydrogen, —(CH₂)ₛOH, —(CH₂)ᵣCO₂H, —(CH₂)ᵣCO₂C₁₋₁₀alkyl, —(CH₂)ᵣCONR^{c}R^{d}, —S(O)_{q}(CH₂)ₚaryl, —(CH₂)ₚaryl, and —(CH₂)ₚheteroaryl, wherein CH₂, alkyl, aryl and heteroaryl are unsubstituted or substituted with one, two or three substituents independently selected from R^{a}; R², R³, R⁴, R⁵, R⁶ and R¹⁰ are each hydrogen; R⁷ and R⁹ are independently selected from the group consisting of: —O—C₁₋₁₀alkyl, and —O—C₃₋₁₀cycloalkyl; R⁸ is selected from the group consisting of: halogen, aryl, and heteroaryl, wherein aryl and heteroaryl are unsubstituted or substituted with one, two or three substituents independently selected from R^{b}; each R^{a} is independently selected from the group consisting of: —OH, —CN, —OC₁₋₆alkyl, halogen, —S(O)₂C₁₋₆alkyl, —CO₂H, —CO₂C₁₋₆alkyl, —C(O)NR^{c}R^{d}, and heteroaryl, wherein alkyl and heteroaryl are unsubstituted or substituted with 1 or 2 substituents selected from oxo, C₁₋₆alkyl, —CO₂H, —NH₂, NH(C₁₋₆alkyl), and NH(C₁₋₆alkyl)₂; and each R^{b} is independently selected from the group consisting of: —CN, halogen, —CF₃, —OCF₃, —C₁₋₆alkyl, —OC₁₋₆alkyl, and —C(O)NR^{c}R^{d}; or a pharmaceutically acceptable salt thereof.

In another embodiment of the present invention are provided compounds of formula I wherein: R¹ is selected from the group consisting of: hydrogen, phenyl and pyridine, wherein phenyl and pyridine are unsubstituted or substituted with one substituent independently selected from R^{a}; R², R³, R⁴, R⁵, R⁶ and R¹⁰ are each hydrogen; R⁷ and R⁹ are independently selected from the group consisting of: —O—CH₂CH₃, and —O-cyclopropyl; R⁸ is selected from the group consisting of: phenyl, and pyridine, wherein phenyl and pyridine are unsubstituted or substituted with one or two substituents independently selected from R^{b};

each R^{a} is independently selected from the group consisting of: —CO₂H, —C(O)NH₂, tetrazole, and oxo-dihydro-oxadiazole; each R^{b} is independently selected from the group consisting of halogen; or a pharmaceutically acceptable salt thereof.

In another embodiment of the present invention, the invention relates to compounds of structural formula Ia:

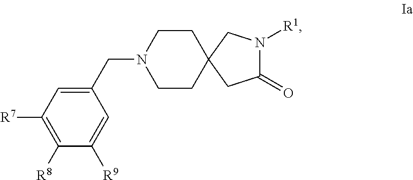

or pharmaceutically acceptable salts thereof.

In another embodiment of the present invention, the invention relates to compounds of structural formula Ib:

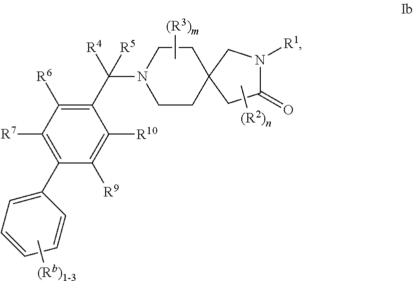

or pharmaceutically acceptable salts thereof.

In another embodiment of the present invention, the invention relates to compounds of structural formula Ic;

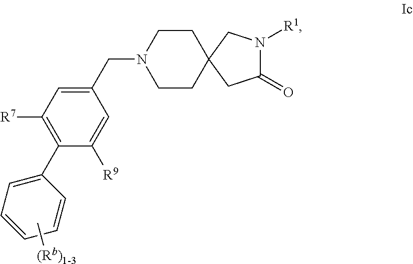

or pharmaceutically acceptable salts thereof.

In another embodiment of the present invention, the invention relates to compounds of structural formula Id:

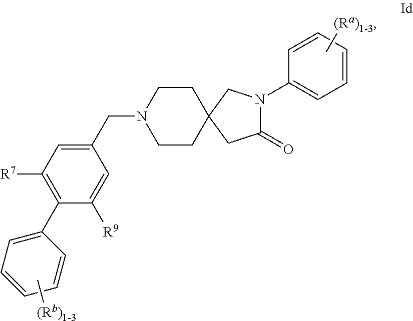

or pharmaceutically acceptable salts thereof.

Illustrative, but nonlimiting examples, of the compounds of the present invention that are useful as antagonists of SSTR5 are the following substituted spirocyclic amines:

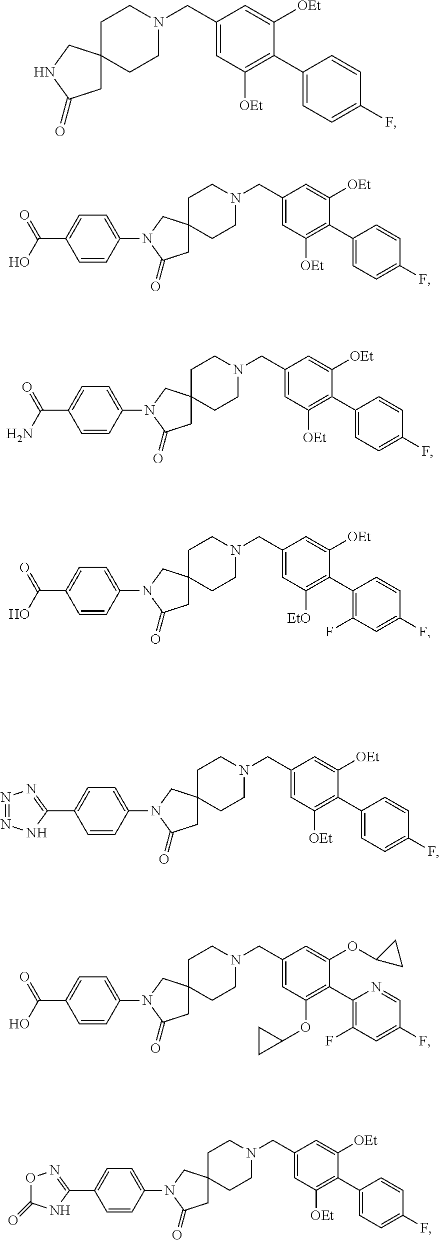

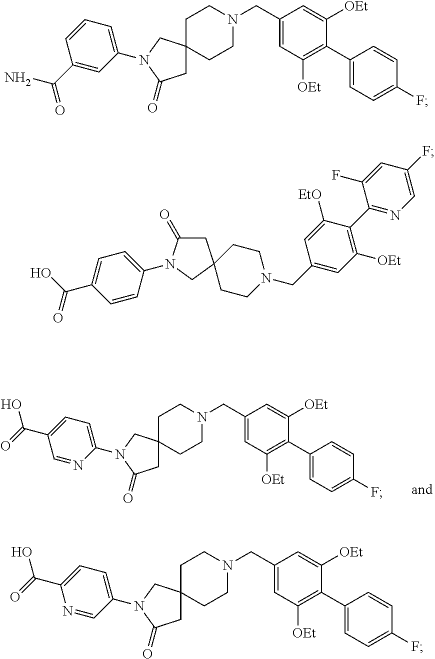

or a pharmaceutically acceptable salt thereof.

### DEFINITIONS

“Alkyl”, as well as other groups having the prefix “alk”, such as alkoxy, alkanoyl, means carbon chains which may be linear or branched or combinations thereof, Examples of alkyl groups include methyl, ethyl, propyl, isopropyl, butyl, sec- and tert-butyl, pentyl, hexyl, heptyl, octyl, nonyl, and the like. The term “Et” means ethyl or —CH₂CH₃. The term “OEt” means ethoxy or —OCH₂CH₃.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Carboxylic acid” or “Carboxylic acid group” means —CO₂H.

“Cycloalkyl” means mono- or bicyclic or bridged saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic cycloheteroalkyl group in which the point of attachment is on the aromatic portion. Examples of aryl include phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” means an aromatic or partially aromatic heterocycle that contains at least one ring heteroatom selected from O, S and N. “Heteroaryl” thus includes heteroaryls fused to other kinds of rings, such as aryls, cycloalkyls and heterocycles that are not aromatic. Examples of heteroaryl groups include pyrrolyl, isoxazolyl, isothiazolyl, pyrazolyl, pyridyl (pyridinyl), oxazolyl, oxadiazolyl (in particular, 1,3,4-oxadiazol-2-yl and 1,2,4-oxadiazol-3-yl), oxo-dihydro-diazole, oxadiazolone, thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, 1,3-benzodioxolyl, benzo-1,4-dioxanyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For cycloheteroalkyl and heteroaryl groups, rings and ring systems containing from 3-15 atoms are included, forming 1-3 rings.

“Cycloheteroalkyl” means mono- or bicyclic or bridged saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “cycloheteroalkyl” include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. The cycloheteroalkyl ring may be substituted on the ring carbons and/or the ring nitrogens.

“Halogen” includes fluorine, chlorine, bromine and iodine.

By “oxo” is meant the functional group “═O” which is an oxygen atom connected to the molecule via a double bond, such as, for example, (1) “C═(O)”, that is a carbonyl group; (2) “S═(O)”, that is, a sulfoxide group; and (3) “N═(O)”, that is, an N-oxide group, such as pyridyl-N-oxide.

When any variable (e.g., R¹, R^{a}, etc.) occurs more than one time in any constituent or in formula I, its definition on each occurrence is independent of its definition at every other occurrence. Also, combinations of substituents and/or variables are permissible only if such combinations result in stable compounds.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

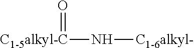

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

### Optical Isomers—Diastereoisomers—Geometric Isomers—Tautomers:

Compounds of structural formula I may contain one or more asymmetric centers and can thus occur as racemates and racemic mixtures, single enantiomers, diastereoisomeric mixtures and individual diastereoisomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer or isomers of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of structural formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within structural formula I, can be prepared without undue experimentation by conventional techniques well known to those skilled in the art or by processes analogous to those described in the Schemes and Examples herein using appropriate isotopically-enriched reagents and/or intermediates.

### Salts:

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

Solvates, and in particular, the hydrates of the compounds of structural formula I are included in the present invention as well.

Exemplifying the invention is the use of the compounds disclosed in the Examples and herein.

### Utilities:

The present invention relates to methods for the treatment, control, or prevention of diseases that are responsive to antagonism of SSTR5. The compounds described herein are potent and selective antagonists of the SSTR5. The compounds are efficacious in the treatment of diseases that are modulated by SSTR5 ligands, which are generally antagonists.

One or more of the following diseases may be treated by the administration of a therapeutically effective amount of a compound of Formula I, or a pharmaceutically acceptable salt thereof, to a subject in need thereof: (1) Type 2 diabetes (also known as non-insulin dependent diabetes mellitus, or NIDDM), (2) hyperglycemia, (3) impaired glucose tolerance, (4) insulin resistance, (5) obesity, (6) lipid disorders, (7) dyslipidemia, (8) hyperlipidemia, (9) hypertriglyceridemia, (10) hypercholesterolemia, (11) low HDL levels, (12) high LDL levels, (13) atherosclerosis and its sequelae, (14) vascular restenosis, (15) abdominal obesity, (16) retinopathy, (17) metabolic syndrome, (18) high blood pressure (hypertension), (19) mixed or diabetic dyslipidemia, and (20) hyperapoBlipoproteinemia.

The present invention also relates to methods for the treatment, control, or prevention of diseases, including but not limited to, diabetes, hyperglycemia, insulin resistance, obesity, lipid disorders, atherosclerosis, and metabolic syndrome by administering, to a subject, the compounds and pharmaceutical compositions described herein. Also, the compounds of Formula I may be used for the manufacture of a medicament for treating one or more of these diseases.

One embodiment of the uses of the compounds is directed to the treatment of one or more of the following diseases by administering a therapeutically effective amount to a subject in need of treatment: type 2 diabetes; insulin resistance; hyperglycemia; lipid disorders; metabolic syndrome; obesity; and atherosclerosis.

The 3-oxo-2,8-diazaspiro[4.5]dec-2-yl compounds of structural formula (I) have the unexpected benefit of increased binding potency (lower Ki) for the hSSTR5 receptor relative to compounds with alternative spirocycle cores. Additionally, the compounds of structural formula (I) have the unexpected benefit of significantly diminished potency on the hERG ancillary ion channel, and this lower potency for blocking hERG reduces the potential for prolongation of the QT interval associated with causing the sometimes fatal ventricular arrhythmia known as torsades de pointer. Finally, the compounds of structural formula (I) have the unexpected benefit of maintaining lower blocking activity on the L-type calcium channel, as measured by the inhibitory potency of the Cav1.2 calcium channel, thereby reducing any undesirable lowering of arterial blood pressure.

The compounds may be used for manufacturing a medicament for use in the treatment of one or more of these diseases.

The compounds are expected to be effective in lowering glucose and lipids in diabetic patients and in non-diabetic patients who have impaired glucose tolerance and/or are in a pre-diabetic condition. The compounds may ameliorate hyperinsulinemia, which often occurs in diabetic or pre-diabetic patients, by modulating the swings in the level of serum glucose that often occurs in these patients. The compounds may also be effective in treating or reducing insulin resistance. The compounds may be effective in treating or preventing gestational diabetes.

The compounds, compositions, and medicaments as described herein may also be effective in reducing the risks of adverse sequelae associated with metabolic syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

By keeping hyperglycemia under control, the compounds may also be effective in delaying or preventing vascular restenosis and diabetic retinopathy.

The compounds of this invention may also have utility in improving or restoring β-cell function, so that they may be useful in treating type 1 diabetes or in delaying or preventing a patient with Type 2 diabetes from needing insulin therapy.

One aspect of the invention provides a method for the treatment and control of mixed or diabetic dyslipidemia, hypercholesterolemia, atherosclerosis, low HDL levels, high LDL levels, hyperlipidemia, and/or hypertriglyceridemia, which comprises administering to a patient in need of such treatment a therapeutically effective amount of a compound having formula I. The compound may be used alone or advantageously may be administered with a cholesterol biosynthesis inhibitor, particularly an HMG-CoA reductase inhibitor such as lovastatin, simvastatin, rosuvastatin, pravastatin, fluvastatin, atorvastatin, rivastatin, itavastatin, or ZD-4522. The compound may also be used advantageously in combination with other lipid lowering drugs such as cholesterol absorption inhibitors (for example stanol esters, steml glycosides such as tiqueside, and azetidinones such as ezetimibe), ACAT inhibitors (such as avasimibe), CETP inhibitors (for example torcetrapib and those described in published applications WO2005/100298, WO2006/014413, and WO2006/014357), niacin and niacin receptor agonists, bile acid sequestrants, microsomal triglyceride transport inhibitors, and bile acid reuptake inhibitors. These combination treatments may be effective for the treatment or control of one or more related conditions selected from the group consisting of: hypercholesterolemia, atherosclerosis, hyperlipidemia, hypertriglyceridemia, dyslipidemia, high LDL, and low HDL.

The term “diabetes” as used herein includes both insulin-dependent diabetes (that is, also known as IDDM, type-1 diabetes), and insulin-independent diabetes (that is, also known as NIDDM, type-2 diabetes).

Diabetes is characterized by a fasting plasma glucose level of greater than or equal to 126 mg/dl. A diabetic subject has a fasting plasma glucose level of greater than or equal to 126 mg/dl. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

The compounds and compositions described herein are useful for treatment of both type 1 diabetes and type 2 diabetes. The compounds and compositions are especially useful for treatment of type 2 diabetes. The compounds and compositions described herein are especially useful for treatment and/or prevention of pre-diabetes. Also, the compounds and compositions described herein are especially useful for treatment and/or prevention of gestational diabetes mellitus.

Treatment of diabetes mellitus refers to the administration of a compound or combination described herein to treat a diabetic subject. One outcome of the treatment of diabetes is to reduce an increased plasma glucose concentration. Another outcome of the treatment of diabetes is to reduce an increased insulin concentration. Still another outcome of the treatment of diabetes is to reduce an increased blood triglyceride concentration. Still another outcome of the treatment of diabetes is to increase insulin sensitivity. Still another outcome of the treatment of diabetes may be enhancing glucose tolerance in a subject with glucose intolerance. Still another outcome of the treatment of diabetes is to reduce insulin resistance. Another outcome of the treatment of diabetes is to lower plasma insulin levels. Still another outcome of treatment of diabetes is an improvement in glycemic control, particularly in type 2 diabetes. Yet another outcome of treatment is to increase hepatic insulin sensitivity.

Prevention of diabetes mellitus, in particular diabetes associated with obesity, refers to the administration of a compound or combination described herein to prevent or treat the onset of diabetes in a subject in need thereof. A subject in need of preventing diabetes is a prediabetic subject. In certain embodiments the compounds described herein can be useful in the treatment, control or prevention of type 2 diabetes and in the treatment, control and prevention of the numerous conditions that often accompany type 2 diabetes, including metabolic syndrome X, reactive hypoglycemia, and diabetic dyslipidemia. Obesity, discussed below, is another condition that is often found with type 2 diabetes that may respond to treatment with the compounds described herein.

The following diseases, disorders and conditions are related to type 2 diabetes, and therefore may be treated, controlled or in some cases prevented, by treatment with the compounds described herein: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) syndrome X, (24) ovarian hyperandrogenism (polycystic ovarian syndrome), and other disorders where insulin resistance is a component.

Dyslipidemias or disorders of lipid metabolism, include various conditions characterized by abnormal concentrations of one or more lipids (i.e. cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as LDL, VLDL and HDL). Dyslipidemia includes atherogenic dyslipidemia. Hyperlipidemia is associated with abnormally high levels of lipids, LDL and VLDL cholesterol, and/or triglycerides. An outcome of the treatment of dyslipidemia, including hyperlipemia, is to reduce an increased LDL cholesterol concentration. Another outcome of the treatment is to increase a low-concentration of HDL cholesterol. Another outcome of treatment is to decrease very low density lipoproteins (VLDL) and/or small density LDL.

The term “metabolic syndrome”, also known as syndrome X, is defined in the Third Report of the National Cholesterol Education Program Expert Panel on Detection, Evaluation and Treatment of High Blood Cholesterol in Adults (ATP-III). ES. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “obesity” as used herein is a condition in which there is an excess of body fat, and includes visceral obesity, The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians than that in Europeans and Americans. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, impaired glucose tolerance, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the compounds or combinations described herein to reduce or maintain the body weight of an obese subject. One outcome of treatment may be reducing the body weight of an obese subject relative to that subject's body weight immediately before the administration of the compounds or combinations described herein. Another outcome of treatment may be decreasing body fat, including visceral body fat. Another outcome of treatment may be preventing body weight gain. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations described herein to reduce or maintain the body weight of a subject at risk of obesity. One outcome of prevention may be reducing the body weight of a subject at risk of obesity relative to that subject's body weight immediately before the administration of the compounds or combinations described herein. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

The term “subject” is a mammal, including but not limited to a human, cat and dog.

In certain embodiments, the pharmaceutical formulations described herein are useful for the treatment, control, or prevention of obesity and the conditions associated with obesity. Obesity may be due to any cause, whether genetic or environmental. Other conditions associated with obesity include gestational diabetes mellitus and prediabetic conditions such as, elevated plasma insulin concentrations, impaired glucose tolerance, impaired fasting glucose and insulin resistance syndrome. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

Also described herein, are methods of enhancing GLP-1 secretion in a subject by administering, to a subject, the compounds and pharmaceutical compositions described herein. The incretin hormone GLP-1 is believed to have several beneficial effects for the treatment of diabetes mellitus and obesity. GLP-1 stimulates glucose-dependent biosynthesis and secretion of insulin, suppresses glucaon secretion, and slows gastric emptying. Glucagon serves as the major regulatory hormone attenuating the effect of insulin in its inhibition of liver gluconeogenesis and is normally secreted by alpha cells in pancreatic islets in response to falling blood glucose levels. The hormone binds to specific receptors in liver cells that trigger glycogenolysis and an increase in gluconeogenesis through cAMP-mediated events. These responses generate glucose (e.g. hepatic glucose production) to help maintain euglycemia by preventing blood glucose levels from falling significantly. In addition to elevated levels of circulating insulin, type 2 diabetics have elevated levels of plasma glucagon and increased rates of hepatic glucose production. Compounds that can enhance GLP-1 secretion are useful in improving insulin responsiveness in the liver, decreasing the rate of gluconeogenesis and glycogenolysis, and lowering the rate of hepatic glucose output resulting in a decrease in the levels of plasma glucose.

### Administration and Dose Ranges:

Any suitable route of administration may be employed for providing a subject, especially a human, with an effective dose of a compound described herein. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds described herein are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus and/or hyperglycemia or hypertriglyceridemia or other diseases for which compounds described herein are indicated, generally satisfactory results are obtained when the compounds described herein are administered at a daily dosage of from about 0.1 milligram to about 100 milligram per kilogram of animal body weight, preferably given as a single daily dose or in divided doses two to six times a day, or in sustained release form. For most large subjects, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human may be as low as 0.1 mg. In some cases, the daily dose may be as high as 1 gram. The dosage regimen may be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms may also have the same or similar dosages.

### Pharmaceutical Compositions:

Another aspect of the present invention provides pharmaceutical compositions which comprise a compound of Formula I and a pharmaceutically acceptable carrier. The phanitaceutical compositions of the present invention comprise a compound of Formula I or a pharmaceutically acceptable salt as an active ingredient, as well as a pharmaceutically acceptable carrier and unsubstituted or other therapeutic ingredients. The term “pharmaceutically acceptable salts” refers to salts prepared from pharmaceutically acceptable non-toxic bases or acids including inorganic bases or acids and organic bases or acids. A pharmaceutical composition may also comprise a prodrug, or a pharmaceutically acceptable salt thereof, if a prodrug is administered.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions as oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

In some instances, depending on the solubility of the compound or salt being administered, it may be advantageous to faimulate the compound or salt as a solution in an oil such as a triglyceride of one or more medium chain fatty acids, a lipophilic solvent such as triacetin, a hydrophilic solvent (e.g. propylene glycol), or a mixture of two or more of these, also unsubstituted or including one or more ionic or nonionic surfactants, such as sodium lauryl sulfate, polysorbate 80, polyethoxylated triglycerides, and mono and/or diglycerides of one or more medium chain fatty acids. Solutions containing surfactants (especially 2 or more surfactants) will form emulsions or microemulsions on contact with water. The compound may also be formulated in a water soluble polymer in which it has been dispersed as an amorphous phase by such methods as hot melt extrusion and spray drying, such polymers including hydroxylpropylmethylcellulose acetate (HPMCAS), hydroxylpropylmethyl cellulose (HPMCS), and polyvinylpyrrolidinones, including the homopolymer and copolymers.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### Combination Therapy:

Compounds of Formula I may be used in combination with other drugs that may also be useful in the treatment or amelioration of the diseases or conditions for which compounds of Formula I are useful. Such other drugs may be administered, by a route and in an amount commonly used therefor, contemporaneously or sequentially with a compound of Formula I. In the treatment of patients who have Type 2 diabetes, insulin resistance, obesity, metabolic syndrome, and co-morbidities that accompany these diseases, more than one drug is commonly administered. The compounds of this invention may generally be administered to a patient who is already taking one or more other drugs for these conditions. Often the compounds will be administered to a patient who is already being treated with one or more antidiabetic compound, such as metformin, sulfonylureas, and/or PPAR agonists, when the patient's glycemic levels are not adequately responding to treatment.

When a compound of Formula I is used contemporaneously with one or more other drugs, a pharmaceutical composition in unit dosage form containing such other drugs and the compound of Formula I is preferred. However, the combination therapy also includes therapies in which the compound of Formula I and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of Formula I, and either administered separately or in the same pharmaceutical composition, include, but are not limited to:

(a) PPAR gamma agonists and partial agonists, including both glitazones and non-glitazones (e.g. troglitazone, pioglitazone, englitazone, MCC-555, rosiglitazone, balaglitazone, netoglitazone, T-131, LY-300512, LY-818, and compounds disclosed in WO02/08188, WO2004/020408, and WO2004/020409.

(b) biguanides, such as metformin and phenformin;

(c) protein tyrosine phosphatase-1B (PIP-1B) inhibitors;

(d) dipeptidyl peptidase-IV (DPP-4) inhibitors, such as sitagliptin, saxagliptin, vildagliptin, and alogliptin;

(e) insulin or insulin mimetics;

(f) sulfonylureas such as tolbutamide, glimepiride, glipizide, and related materials;

(g) α-glucosidase inhibitors (such as acarbose);

(h) agents which improve a patient's lipid profile, such as (i) HMG-CoA reductase inhibitors (lovastatin, simvastatin, rosuvastatin, pravastatin, fluvastatin, atorvastatin, rivastatin, itavastatin, ZD-4522 and other statins), (ii) bile acid sequestrants (cholestyramine, colestipol, and dialkylaminoalkyl derivatives of a cross-linked dextran), (iii) niacin receptor agonists, nicotinyl alcohol, nicotinic acid, or a salt thereof, (iv) PPARα agonists, such as fenofibric acid derivatives (gemfibrozil, clofibrate, fenofibrate and bezafibrate), (v) cholesterol absorption inhibitors, such as ezetimibe, (vi) acyl CoA:cholesterol acyltransferase (ACAT) inhibitors, such as avasimibe,

(vii) CETP inhibitors, such as torcetrapib, and (viii) phenolic antioxidants, such as probucol;

(i) PPARα/γ dual agonists, such as muraglitazar, tesaglitazar, farglitazar, and JT-501;

(j) PPARδ agonists, such as those disclosed in WO97/28149;

(k) anti-obesity compounds, such as fenfluramine, dexfenfluramine, phentiramine, subitramine, orlistat, neuropeptide Y Y5 inhibitors, MC4R agonists, cannabinoid receptor 1 (CB-1) antagonists/inverse agonists (e.g., rimonabant and taranabant), and β₃ adrenergic receptor agonists;

(l) ileal bile acid transporter inhibitors;

(m) agents intended for use in inflammatory conditions, such as aspirin, non-steroidal anti-inflammatory drugs, glucocorticoids, azulfidine, and cyclooxygenase-2 (Cox-2) selective inhibitors;

(n) glucagon receptor antagonists;

(o) GLP-1;

(p) GIP-1;

(q) GLP-1 analogs and derivatives, such as exendins, (e.g., exenatide and liruglatide);

(r) 11β-hydroxysteroid dehydrogenase-1 (HSD-1) inhibitors;

(s) inhibitors of cholesteryl ester transfer protein (CETP), such as torcetrapib;

(t) SSTR3 antagonists;

(u) other SSTR5 antagonists;

(v) acetyl CoA carboxylase-1 and/or -2 inhibitors;

(w) AMPK activators;

(x) agonists of GPR-119;

(y) glucokinase agonists; and

(z) FGF-21 agonists.

The above combinations include combinations of a compound of the present invention not only with one other active compound, but also with two or more other active compounds. Non-limiting examples include combinations of compounds having Formula I with two or more active compounds selected from biguanides, sulfonylureas, HMG-CoA reductase inhibitors, other PPAR agonists, PIP-1B inhibitors, DPP-4 inhibitors, and cannabinoid receptor 1 (CB1) inverse agonists/antagonists.

Antiobesity compounds that can be combined with compounds described herein include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds described herein, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds described herein include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds described herein include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Suitable melanocortin-4 receptor (MC4R) agonists include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US20031109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474, and WO 03/104761.

The compounds of structural formula I of the present invention can be prepared according to the procedures of the following Schemes, Intermediates and Examples, using appropriate materials and are further exemplified by the following specific examples. Moreover, by utilizing the procedures described in the disclosure contained herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those previously described herein. The use of protecting groups for the amine and carboxylic acid functionalities to facilitate the desired reaction and minimize undesired reactions is well documented. Conditions required to remove protecting groups are found in standard textbooks such as Greene, T, and Wuts, P. G. M., Protective Groups in Organic Synthesis, John Wiley & Sons, Inc., New York, N.Y., 1991. CBZ and BOC are commonly used protecting groups in organic synthesis, and their removal conditions are known to those skilled in the art.

Reactions sensitive to moisture or air were performed under nitrogen or argon using anhydrous solvents and reagents. The progress of reactions was determined by either analytical thin layer chromatography (TLC) or liquid chromatography-mass spectrum (LC-MS). Concentration of solutions was carried out on a rotary evaporator under reduced pressure. ¹H NMR spectra were acquired on a 500 MHz Varian Unity INOVA NMR spectrometer in CDCl₃ solutions unless otherwise noted. Chemical shifts were reported in parts per million (ppm). Tetramethylsilane (TMS) was used as internal reference in CD₃Cl solutions, and residual CH₃OH peak or TMS was used as internal reference in CD₃OD solutions. Coupling constants (J) were reported in hertz (Hz). All temperatures are degrees Celsius unless otherwise noted. Mass spectra (MS) were measured by electron-spray ion-mass spectroscopy,

### Abbreviations Used in the Following Schemes and Examples:

aq.: aqueous; API-ES: atmospheric pressure ionization-electrospray (mass spectrum term); Ac: acetate; AcCN: acetonitrile; Bop reagent: (benzotriazol-1-yloxy)tris(dimethylamino)phosonium hexafluorophosphate; Boc: tert-butyloxycarbonyl; B(OTMS)₃: tris(trimethylsilyl) borate; Celite™: diatomaceous earth; CDI: carbonyl diimidazole; d: day(s); d is doublet (NMR); DCM: dichloromethane; Dess-Martin reagent: 1,1,1-triacetoxy-1,1-dihydro-1,2-benziodoxol-3(1H)-one; DIBAL: diisobutylaluminum hydride; DIEA and DIPEA: N,N-diisopropyl-ethylamine (Hunig's base); DMAP: 4-dimethylaminopyridine; DMF: N,N-dimethylformamide; DMSO: dimethylsulfoxide; DTBPF is 1,1′-bis(di-tert-butylphosphino)-ferrocene; eq: equivalent(s); Et is ethyl; OEt is ethoxy; EtOAc: ethyl acetate; EtOH: ethanol; g: gram(s); h or hr: hour(s); HPLC: high pressure liquid chromatography; HPLC/MS: high pressure liquid chromatography/mass spectrum; in vacuo: rotary evaporation under diminished pressure; iPrOH or IPA: isopropyl alcohol; IPAC or IPAc: isopropyl acetate; [Ir(COD)Cl]₂: chloro-1,5-cyclooctadiene iridium (I) dimmer; L: liter; LC: Liquid chromatography; LC-MS: liquid chromatography-mass spectrum; m is multiplet (NMR); M: molar; Me: methyl; MeCN: methylcyanide; MeI: methyl iodide; MeOH: methanol; Ms: methanesulfonyl; MsCl: methanesulfonyl chloride; MHz: megahertz; mg: milligram; min: minute(s); ml or mL: milliliter; mmol: millimole; MPLC: medium-pressure liquid chromatography; MS or ms: mass spectrum; N: normal; nM: nanomole(s); NMR: nuclear magnetic resonance; NMM: N-methylmorpholine; Pd₂(dba)₃: tris(dibenzyldeneacetone)dipalladium(0); q is quadruplet (NMR); Rₜ: retention time; it or RT: room temperature; s is singlet (NMR); satd.: saturated; SRIF is somatotropin release-inhibiting factor or somatostatin; t is triplet (NMR); TBAF is tetrabutyl ammonium fluoride; TBS is tert-butyldimethylsilyl; TBSCl is tert-butyldimethylsily chloride; TEA: triethylamine; TFA: trifluoroacetic acid; THE: tetrahydrofuran; TLC or tlc: thin layer chromatography; Tf is trifluoromethane sulfonate; and Ts is toluene sulfonyl.

The present compounds can be prepared according to the general Schemes provided below as well as the procedures provided in the Examples. The following Schemes and Examples further describe, but do not limit, the scope.

Scheme 1 illustrates the synthesis of Chloride 4. Commercially available 4-bromo-3,5-dihydroxybenzoic acid was treated with excess iodoethane and K₂CO₃ to give bromide 1. Bromide 1 underwent a Suzuki coupling reaction with an appropriate aryl and heteroaryl boronic acid to give ethyl ester 2.

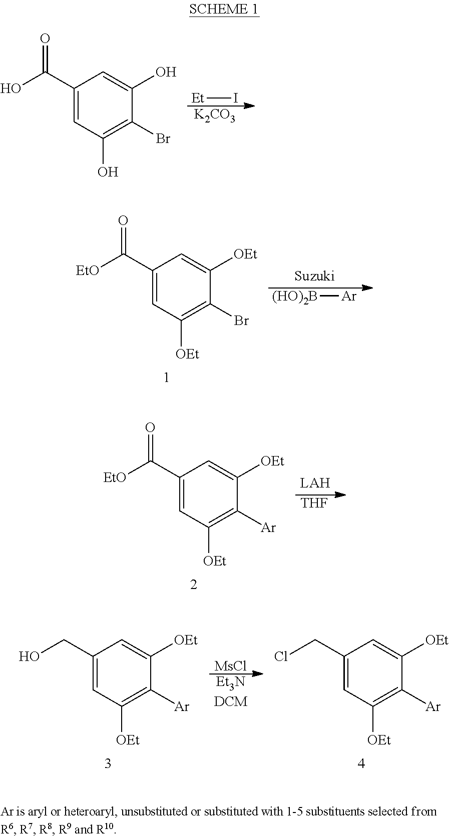

Ethyl ester 2 was reduced with LAH to give corresponding alcohol 3. Alcohol 3 was treated with MsCl and TEA to give the corresponding mesylate. However, the mesylate intermediate was not isolated. Under the reaction conditions, the mesyl group was displaced by chloride to give the more stable chloride 4.

### Intermediate 1

### 4-(Chloromethyl)-2,6-diethoxy-4′-fluorobiphenyl

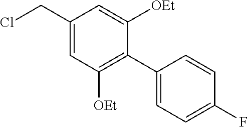

### Step A: Synthesis of Ethyl 4-bromo-3,5-diethoxybenzoate

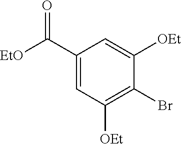

Iodoethane (17.3 mL, 215 mmol) was added to a stirred mixture of 4-bromo-3,5-dihydroxybenzoic acid (10 g, 42.9 mmol) and potassium carbonate (26.7 g, 193 mmol). The mixture was stirred at room temperature for 18 hours, and then partitioned between EtOAc and water. The aqueous phase was extracted with EtOAc. The combined organic phases were washed with water (2×), brine, dried (MgSO₄) and concentrated to give the title compound. H NMR (500 MHz, CDCl₃, ppm): 1.4 (t, 3H), 1.5 (t, 6H), 4.2 (q, 4H), 4.4 (q, 2H), 7.2 (s, 2H).

### Step B: Synthesis of Ethyl 2,6-diethoxy-4′-fluorobiphenyl-4-carboxylate

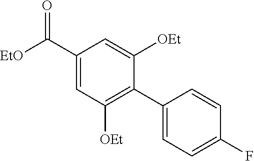

Dioxane (120 mL) was added to a degassed mixture of tri-t-butylphosphonium tetrafluoroborate (0.73 g, 2.5 mmol) 4-fluorophenylboronic acid (11.8 g, 84 mmol) tris(dibenzylideneacetone)dipalladium(0) (0.77 g, 0.84 mmol), CsF (23.7 g, 156 mmol) and ethyl 4-bromo-3,5-diethoxybenzoate (Step A, 13.4 g, 42 mmol). The mixture was stirred at 90° C. under nitrogen for 20 hours, and then partitioned between EtOAc and water. The aqueous phase was filtered and extracted with EtOAc. The combined organic phases were washed with water, brine, dried (MgSO₄), and concentrated. The resulting residue was chromatographed on silica gel columns by eluting with EtOAc/hexane. Product fractions were combined and concentrated to give the title compound. LC-MS, M+1=333.1; H NMR (500 MHz, CDCl₃, ppm): 1.3 (t, 6H), 1.45 (t, 3H), 4.1 (q, 4H), 4.4 (q, 2H), 7.1 (m, 2H), 7.2 (s, 2H), 7.4 (m, 2H).

### Step C: Synthesis of (2,6-Diethoxy-4′-fluorobiphenyl-4-yl)methanol

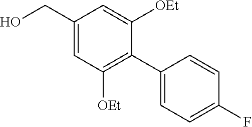

A solution of lithium alumiumhydride (34 ml, 34 mmol) in THF was added dropwise over 30 min to a stirred solution of ethyl 2,6-diethoxy-4′-fluorobiphenyl-4-carboxylate (Step B, 14 g, 42 mmol) in THF (120 mL) at room temperature. After 2 hours at room temperature, the reaction mixture was refrigerated overnight and then quenched by the sequential addition of water (4 mL), aqueous NaOH (0.5 M, 4 mL), and water (4 mL). The mixture was filtered through Celite™, washed thoroughly with EtOAc, and concentrated to give the title compound as white solid. H NMR (500 MHz, CDCl₃, ppm): 1.3 (t, 6H), 1.8 (t, 1H), 4.0 (q, 4H), 4.7 (d, 2H), 6.7 (s, 2H), 7.1 (m, 2H), 7.4 (m, 2H).

### Step D: Synthesis of 4-(Chloromethyl)-2,6-diethoxy-4′-fluorobiphenyl

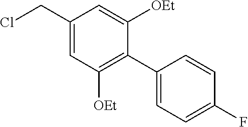

Methanesulfonyl chloride (1.6 mL, 20.7 mmol) was added dropwise to a stirred solution of (2,6-Diethoxy-4′-fluorobiphenyl-4-yl)methanol (Step C, 5 g, 17.2 mmol) and triethylamine (3.6 mL, 25.8 mmol). The mixture was stirred at room temperature for 18 hours, and then partitioned between EtOAc and water. The aqueous phase was extracted with EtOAc. The combined organic phases were washed with water, brine, dried (MgSO₄) and concentrated. The resulting residue was chromatographed on a silica gel column by eluting with EtOAc/hexane. The product fractions were combined and concentrated to give the title compound. ¹H NMR (500 MHz, CDCl₃, ppm): 1.3 (t, 6H), 4.0 (q, 4H), 4.6 (s, 2H), 6.7 (s, 2H), 7.1 (m, 2H), 7.4 (m, 2H). LC-MS, m+1=309.1.

Scheme 2 illustrates the synthesis of Aldehyde 12. Commercially available 4-bromo-3,5-dihydroxybenzoic acid underwent a vinyl exchange reaction (Okimoto et al., Journal of the American Chemical Society, 124, 1590-1591; 2002) with vinylacetate catalyzed by [Ir(COD)Cl]₂ to give vinyl ether 6. Vinyl ether 6 was converted to corresponding cyclopropyl ether compound 7 under standard cyclopropanation condition (Shi et al., Tetrahedron Letters 39, 8621-8624, 1998). Ester group of compound 7 was reduced to give alcohol 8, and the resulting alcohol was protected as a TBS ether by treatment with TBSCl to give TBS ether 9.

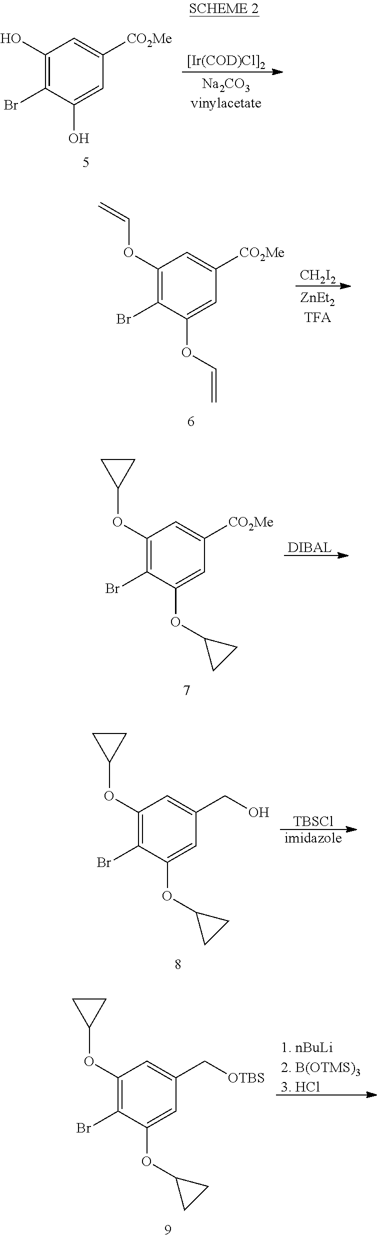

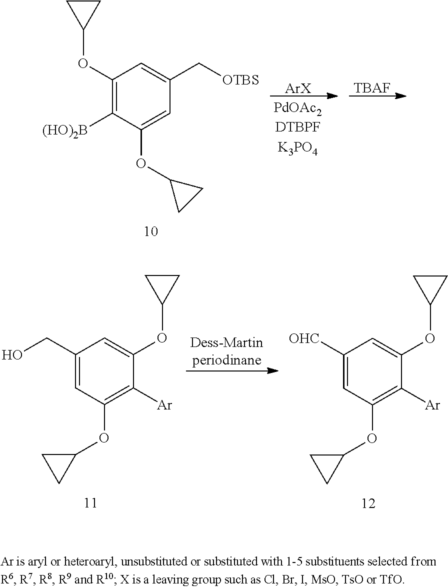

To expand the Suzuki coupling reaction substrate scope (for the substrates for which aryl boronic acids are not commercially available or not stable), TBS ether 9 was converted to the boronic acid derivative 10. The boronic acid derivative 10 underwent Suzuki coupling with the appropriate aryl halides, followed by the removal of the TBS protecting group to give alcohol 11. Alcohol 11 was treated with Dess-Martin periodinane reagent to give corresponding aldehyde 12.

### Intermediate 2

### 3,5-Bis(cyclopropyloxy)-4-(3,5-difluoropyridin-2-yl)benzaldehyde

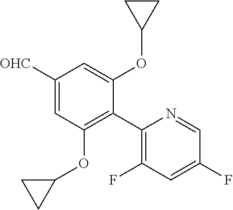

### Step A: Synthesis of Methyl 4-bromo-3,5-bis(ethenyloxy)benzoate

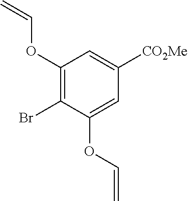

To a nitrogen flushed round bottom flask was added methyl 4-bromo-3,5-dihydroxy-benzoate (5 g, 20.2 mmol), vinyl acetate (7.0 g, 81 mmol), sodium carbonate (2.6 g, 24.3 mmol), chloro(1,5-cyclooctadiene)iridium (I) dimer (0.136 g, 0.20 mmol) and toluene (20 ml). The resulting reaction mixture was heated at 105° C. for 16 hours under a nitrogen atmosphere. After cooling to ambient temperature, the reaction mixture was diluted with 50 mL ether, and washed with 50 mL KOH (5%). The organic layer was separated, dried over Na₂SO₄, filtered and concentrated. The resulting residue was purified by silica gel chromatography by eluting with a gradient of hexane to 1:9 hexane/ethyl acetate to give the title compound as white solid. ¹H-NMR (CDCl₃): [symbol] : 7.47 (s, 2H), 6.67 (dd, 13.6, 6.1 Hz, 1H), 4.92 (dd, J=13.7, 2.0 Hz, 1H), 4.65 (dd, J=6.1, 2.1 Hz, 1H), 3.95 (s, 3H).

### Step B: Synthesis of Methyl 4-bromo-3,5-bis(cyclopropyloxy)benzoate

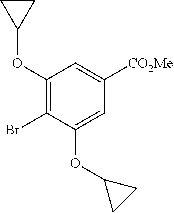

To a 250 mL nitrogen flushed round bottom flask was added 120 mL of methylene chloride, and diethylzinc (13 ml, 1 M solution in CH₂Cl₂). Then TFA (0.97 ml, 12.5 mmol) was slowly added via syringe at 0° C. The resulting reaction mixture was stirred at 0° C. for 10 minutes, followed by the addition of diiodomethane (1.09 ml, 13.5 mmol). The resulting clear solution was stirred at 0° C. for 10 more minutes, then a solution of methyl 4-bromo-3,5-bis(ethenyloxy)benzoate (0.75 g, 2.5 mmol) in 10 mL methylene chloride was added. The reaction mixture was allowed to warm up to room temperature and stirred at room temperature overnight. The reaction mixture was then quenched by addition of 100 mL of saturated NH₄Cl. The organic layer was separated, dried over Na₂SO₄, filtered and concentrated. The resulting crude product was purified by silica gel chromatography by eluting with a gradient of: hexane to 1:10 hexane/ethyl acetate to give the title compound as white solid. ¹H-NMR (CDCl₃): [symbol] : 7.60 (s, 2H), 3.96 (s, 3H), 3.90 (m, 2H), 0.88 (m, 8H).

### Step C: Synthesis of {[4-Bromo-3,5-bis(cyclopropyloxy)benzyl]oxy}(tert-butyl)dimethylsilane

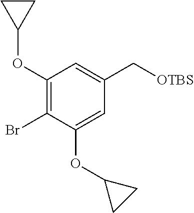

To a round bottom flask was added methyl 4-bromo-3,5-bis(cyclopropyloxy)benzoate and 10 mL ether, followed by the addition of DIBAL at 0° C. The resulting reaction mixture was stirred at 0° C. for 10 minutes, then 20 mL EtOAc were added, followed by the addition of 30 mL of 1 N HCl. The organic layer was separated, washed with 10 mL brine, dried over sodium sulfate, filtered and concentrated. The resulting crude product was dissolved in 10 mL of EtOAc. Then imidazole (205 mg, 3.0 mmol) and TBSCl (302 mg, 2.0 mmol) were added. The resulting reaction mixture was stirred at room temperature overnight, then diluted with 20 mL ether, and washed with 2×30 mL of saturated NH₄Cl. The organic layer was separated, dried over sodium sulfate, filtered and concentrated. The resulting crude product was purified by silica gel chromatography by eluting with a gradient of hexane to 1:9 hexane/ethyl acetate to give the title compound as colorless oil. ¹H-NMR (CDCl₃): [symbol] : 6.95 (s, 2H), 4.76 (s, 2H), 3.81 (m, 2H), 0.98 (m, 9H), 0.8 (m, 8H), 0.14 (s, 6H).

### Step D: Synthesis of 2-[4-({[tert-Butyl(dimethyl)silyl]oxy}methyl)-2,6-bis(cyclo-propyloxy)phenyl]-3,5-difluoropyridine

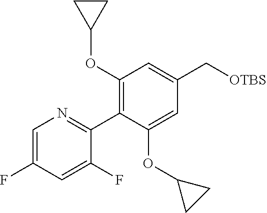

To a nitrogen flushed vial was added {[4-bromo-3,5-bis(cyclopropyloxy)benzyl]-oxy}(tert-butyl)dimethylsilane (170 mg, 0.41 mmol) and 2 mL THF, followed by the addition of nBuLi (0.18 ml, 2.5 M in hexane) at −78 C. The resulting reaction mixture was stirred at −78° C. for 5 minutes, then tris(trimethylsilyl) borate (147 mg, 0.49 mmol) was added via a syringe. The reaction mixture was allowed to warm up to room temperature and quenched by addition of 5 mL of Na₂CO₃. The resulting mixture was diluted with 20 mL EtOAc, and acidified to pH 1 with 3 N HCl. The organic layer was separated, washed with 15 mL brine, dried over sodium sulfate, filtered and concentrated. The resulting crude product (120 mg, 0.32 mmol) was added to a microwave tube, followed by the addition of 2-Bromo-3,5-difluoropyridine (74 mg, 0.38 mmol), K₃PO₄ (202 mg, 0.95 mmol), PdOAc₂ (7.1 mg, 0.032 mmol), DTBPF (15 mg, 0.032 mmol) and 2 mL THF. The microwave tube was sealed, and the reaction mixture was flushed with nitrogen and heated at 80° C. overnight. After cooling to room temperature, the reaction mixture was diluted with EtOAc. After an aqueous work up, the crude product was purified via silica gel chromatography by eluting with a gradient of hexane to 1:9 hexane/ethyl acetate as the eluent to give the title compound as light brown viscous material. ¹H-NMR (CDCl₃): b: 8.47 (m, 1H), 7.3 (m, 1H), 7.03 (s, 1H), 4.84 (s, 1H), 3.75 (m, 2H), 1.10 (s, 9H), 0.7 (m, 8H), 0.174 (s, 6H).

### Step E: Synthesis of 3,5-Bis(cyclopronyloxy)-4-(3,5-difluoropyridin-2-yl)benzaldehyde

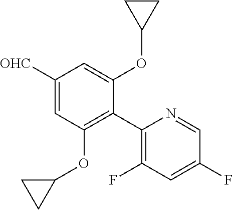

To a solution of 2-[4-({[tert-butyl(dimethyl)silyl]oxy}methyl)-2,6-bis(cyclopropyloxy)-phenyl]-3,5-difluoropyridine (82 mg, 0.18 mmol) in 2 mL EtOAc was added TBAF (0.55 ml, 1 M THF solution). The resulting clear solution was stirred at room temperature for 1 hour, then diluted with EtOAc. After an aqueous work up, the resulting crude product was purified on prep-TLC eluted with 1:1 EtOAc/hexane to give the desired alcohol compound. The alcohol compound was then dissolved in 3 mL of CH₂Cl₂, followed by the addition of Dess-Martin reagent. The resulting reaction mixture was stirred at room temperature for 1 hour, then diluted with 15 mL of ether, washed with 2×20 mL of Na₂CO₃. The organic layer was separated, dried over sodium sulfate, filtered and concentrated to give the title compound. ¹H-NMR (CDCl₃): b: [symbol] : 10.1 (s, 1H), 8.44 (d, J=2.4 Hz, 1H), 7.53 (s, 2H), 7.25 (m, 1H), 3.83 (m, 2H), 0.75 (m, 8H). MS (m/e): 332 (M+1).

The compounds of general structure 17 described in this application were synthesized according to Scheme 3. Commercially available tert-butyl 3-oxo-2,8-diazaspiro[4.5]decane-8-carboxylate was either subjected to Buchwald-Hartwig C—N coupling reaction with the appropriate aryl or heteroaryl halide, or subjected to a base promoted alkylation or sulfonylation to give compound 13.

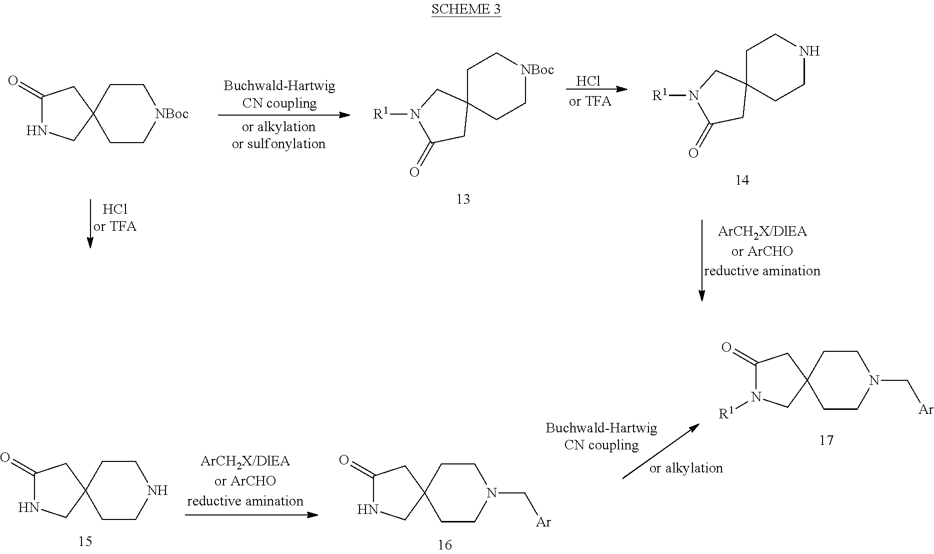

Ar is aryl or heteroaryl, unsubstituted or substituted with 1 to 5 substituents selected from R⁶, R⁷, R⁸, R⁹ and R¹⁰; and X is a leaving group such as Cl, Br, I, MsO, TsO or TfO.

The Boe protecting group of compound 13 may be removed under strong acidic conditions to give amine 14 as the HCl or TFA salt. Amine 14 is then subjected to alkylation with appropriate reagent or is subjected to reductive amination with appropriated aldehyde or ketone to give the desired compound 17 using methods known to those skilled in the art. Alternatively, tert-butyl 3-oxo-2,8-diazaspiro[4.5]decane-8-carboxylate may be first treated with strong acid to give amine 15 as the HCl or TFA salt, which undergoes alkylation with appropriate halide or was subjected to reductive amination with appropriated aldehyde or ketone to give compound 16. Compound 16 is either subjected to Buchwald-Hartwig C—N coupling reaction with appropriated aryl or heteroaryl halide, or undergoes base promoted direct alkylation to give the desired compound 17.

### Intermediate 3

### Methyl 4-(3-oxo-2,8-diazaspiro[4.5]dec-2-yl)benzoate hydrochloride

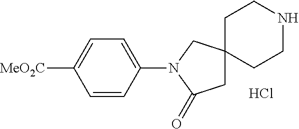

### Step A: Synthesis of tert-Butyl-2-[4-(methoxycarbonyl)phenyl]-3-oxo-2,8-diazaspiro-[4.5]decane-8-carboxylate

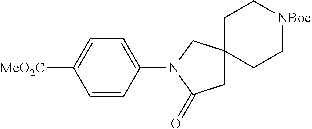

To a round bottom flask was added tert-butyl 3-oxo-2,8-diazaspiro[4.5]decane-8-carboxylate (3 g, 11.8 mmol, Pharmaron); methyl 4-bromobenzoate (3.8 g, 17.7 mmol); K₃PO₄ (7.5 g, 35.4 mmol); Pd₂(dba)₃ (0.108 g, 0.118 mmol); 9,9-dimethyl-4,5-bis(diphenylphosphino)-xanthene (Xantaphos) (0.137 g, 0.236 mmol); and 15 mL dioxane. The reaction mixture was thoroughly degassed with nitrogen, and heated at 100° C. overnight. After cooling to room temperature, reaction mixture was diluted with a mixture of 75 mL EtOAc/75 mL ether, and washed with 120 mL water. The organic layer was separated, dried over sodium sulfate, filtered and concentrated. The resulting crude material was purified via silica gel chromatography by eluting with a gradient of: 1:3 to 2:1 ethyl acetate/hexane to give the title compound as light yellow solid. ¹H-NMR (CDCl₃): [symbol] : 8.06 (d, J=7 Hz, 2H), 7.73 (d, J=8.9 Hz, 2H), 3.93 (s, 3H), 3.71 (s, 2H), 3.64 (b, 2H), 3.35 (m, 2H), 2.59 (s, 2H), 1.71 (m, 4H), 1.49 (s, 9H). Mass Spectra (m/e): 389 (M+1).

### Step B: Synthesis of Methyl 4-(3-oxo-2,8-diazaspiro[4.5]dec-2-yl)benzoate hydrochloride

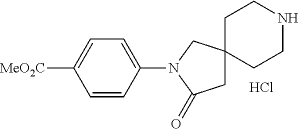

To a solution of tert-butyl-2-[4-(methoxycarbonyl)phenyl]-3-oxo-2,8-diazaspiro-[4.5]decane-8-earboxylate (2.4 g, 6.2 mmol) in 10 mL of EtOAc was added HCl in dioxane (9.3 ml, 4 M). The resulting reaction mixture was stirred at room temperature overnight, then diluted with 100 mL hexane, filtered and air dried to give the title compound as light yellow solid. ¹H-NMR (CD₃OD): [symbol] : 8.04 (d, J=7 Hz, 2H), 7.79 (d, J=8.9 Hz, 2H), 3.90 (s, 3H), 3.89 (s, 2H), 3.3 (m, 4H), 3.35 (m, 2H), 2.69 (s, 2H), 1.9 (m, 4H). Mass Spectra (m/e): 289 (M+1).

### Intermediate 4

### 4′-fluoro-2,6-dihydroxybiphenyl-4-carbaldehyde

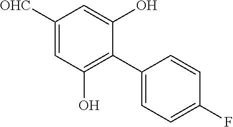

### Step A: Synthesis of benzyl 3,5-bis(benzyloxy)-4-bromobenzoate

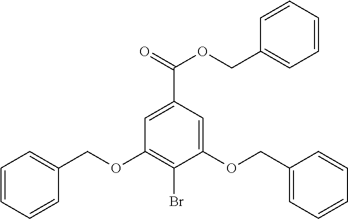

To a round bottom flask, 4-bromo-3,5-dihydroxybenzoic acid (2 g, 8.6 mmol), K₂CO₃ (7.12 g, 51.5 mmol), benzyl bromide (4.1 ml, 34.3 mmol) and 15 ml DMF were added. The resulting reaction mixture was stirred overnight at 50° C. After cooling down to ambient temperature, the reaction mixture was diluted with 100 ml water and 60 ml EtOAc. Layers were separated. Organics were washed with 30 ml brine, dried over sodium sulfate, filtered and concentrated. Residue was recrystallized from EtOAc and hexane to give the desired product as a white solid. ¹H-NMR (CDCl₃): [symbol] : 7.3-7.5 (m, 17H), 5.36 (s, 2H) 5.23 (s, 4H).

### Step B: Synthesis of benzyl 2,6-bis(benzyloxy)-4′-fluorobiphenyl-4-carboxylate

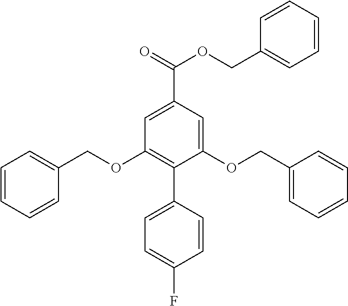

To a 100 ml round bottom flask equipped with a reflux condenser were added benzyl 2,6-bis(benzyloxy)-4′-bromobiphenyl-4-carboxylate (1 g, 1.99 mmol), 4-fluoro-phenyl boronic acid (0.42 g, 2.98 mmol), K₃PO₄ (1.27 g, 5.96 mmol), Pd(dppf)Cl₂ (0.081 g, 0.10 mmol) and 10 ml THF. The resulting reaction mixture was flushed with N₂, and heated at 85° C. overnight. After cooling to ambient temperature, reaction mixture was diluted with 60 ml water and 60 ml EtOAc. Layers were separated. Organics were dried over Na₂SO₄, filtered and concentrated. Crude product was purified on silica gel column eluted with gradient solvent from 100% hexane to 1:4 E/H to give 0.85 g desired product as white solid. ¹H-NMR (CDCl₃): [symbol] : 7.1-7.5 (m, 21H), 5.41 (s, 2H) 5.10 (s, 4H)

### Step C: Synthesis of 2,6-bis(benzyloxy)-4′-fluoro-N-methoxy-N-methylbiphenyl-4-carboxamide

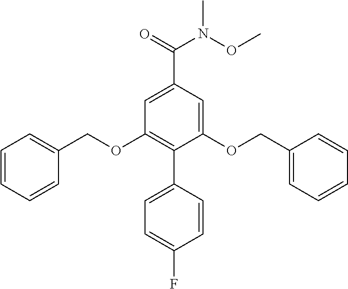

To a 100 ml round bottom flask benzyl 2,6-bis(benzyloxy)-4′-fluorobiphenyl-4-carboxylate (1.14 g, 2.2 mmol), N-methoxymethanamine hydrochloride (0.28 g, 2.86 mmol) and 10 ml THF were added. Isopropylmagnesium chloride in THF (2.75 ml, 2M) was added to the reaction mixture via a syringe at −10° C. The resulting reaction mixture was allowed to warm up to room temperature. The reaction mixture was diluted with 30 ml NH₄Cl (sat.), 30 ml water and 50 ml EtOAc. Layers were separated. Organics were dried over Na₂SO₄, filtered and concentrated. Crude product was purified on silica gel column, eluted with gradient solvent from 1:10 to 1:1 E/H to give the desired product as a colorless solid. ¹H-NMR (CDCl₃): [symbol] : 7.1-7.5 (m, 16H), 5.08 (s, 4H) 3.46 (s, 3H), 3.34 (s, 3H).

### Step D: Synthesis of 4′-fluoro-2,6-dihydroxy-N-methoxy-N-methylbiphenyl-4-carboxamide

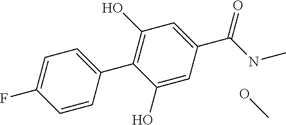

To a 100 ml round bottom flask 2,6-bis(benzyloxy)-4′-fluoro-N-methoxy-N-methylbiphenyl-4-carboxamide (0.64 g, 1.36 mmol), Pd/C (0.072 g, 10%, 0.068 mmol) and 5 ml EtOH were added. The resulting reaction mixture was stirred at 50° C. under balloon H₂ atmosphere for 1 hr. The reaction mixture was diluted with 20 ml EtOAc and filtered through a pad of celite. The filtrate was concentrated. Crude product was recrystallized from EtOAc/hexane to give the desired product as a white solid. ¹H-NMR (CDCl₃): [symbol] : 7.42 (m, 2H), 7.26 (m, 2H), 6.94 (s, 2H), 3.68 (s, 3H) 3.39 (s, 3H).

### Step E: Synthesis of 4′-fluoro-2,6-dihydroxy-biphenyl-4-carbaldehyde

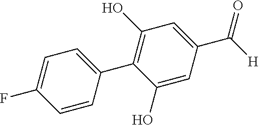

To a 100 ml round bottom flash 4′-fluoro-2,6-dihydroxy-N-methoxy-N-methylbiphenyl-4-carboxamide (100 mg, 0.343 mmol) and 5 ml THF were added. DIBAL in toluene (1.37 ml, 1 M) was added to the reaction mixture at −78° C. The resulting reaction mixture was allowed to warm up to room temperature. EtOAc (20 ml) and wet silica gel (10 g silica gel and 0.5 ml water) were added. The resulting reaction mixture was stirred for 10 min. It was filtered. The filtrate was concentrated. Residue was purified on silica gel column, eluted with gradient solvent from 1:9 to 1:1 E/1-1 to give the desired product as a colorless solid. ¹H-NMR (CDCl₃): [symbol] : 9.86 (s, 1H), 7.43 (m, 2H), 7.22 (m, 2H), 7.09 (s, 2H), 6.41 (s, 2H).

### Intermediate 5

### 4-Ethoxy-2′,3′,4′-trifluorobiphenyl-2-carbaldehyde

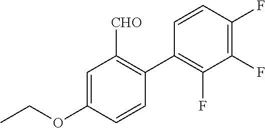

### Step A: Synthesis of 2-Bromo-5-ethoxybenzaldehyde

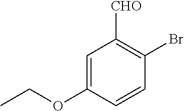

To a solution of 2-bromo-5-hydroxybenzaldehyde (5 g, 24.87 mmol) in DMF (20 mL) K₂CO₃ (6.88 g, 49.7 mmol) was added portion wise. To this, iodoethane (5.82 g, 37.3 mmol) was added slowly and the resulting reaction mixture was stirred overnight at 50° C. After cooling down to ambient temperature, the reaction mixture was diluted with 100 mL ether/hexane(1:1) and 100 mL water. Layers were separated. The organic layer was washed with 100 mL brine, dried over sodium sulfate, filtered and concentrated to give the desired product as a white solid. ¹H-NMR (CDCl₃): [symbol] : 10.3 (s, 1H), 7.51 (d, J=9.0 Hz, 1H), 7.39 (d, J=3.0 Hz, 1H) 7.02 (dd, J=3.5 Hz, J=8.5 Hz, 1H), 4.06 (q, 2H), 1.42 (t, J=6.5 Hz, 3H).

### Step B: Synthesis of 4-Ethoxy-2′,3′,4′-trifluorobiphenyl-2-carbaldehyde

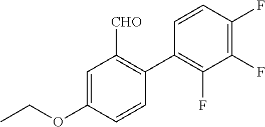

To a degassed solution of 2-bromo-5-ethoxybenzaldehye (300 mg, 1.31 mmol), 2-Dicyclohexylphosphino-2′6′-dimethoxy-1,1′-biphenyl (53.8 mg, 0.131 mmol; S-Phos ligand), palladium(II)acetate (14.7 mg, 0.065 mmol) in THF (8 ml) K₃PO₄ (834 mg, 3.93 mmol) and 2,3,4-trifluorophenyl boronic acid (276 mg, 1.57 mmol) were added. The reaction mixture was stirred at 70° C. under N₂ atmosphere for 16 h. After cooling to room temperature, the reaction mixture was filtered and the filtrate was concentrated by evaporation under reduced pressure. The crude material was purified on CombiFlash column eluting with 5% to 10% EtOAc in hexane to provide 4-ethoxy-2′,3′,4′-trifluorobiphenyl-2-carbaldehyde as a white solid. ¹H-NMR (CDCl₃): [symbol] : 9.88 (s, 1H), 7.53 (s, 1H), 7.30 (m, 1H), 7.24 (dd, J=3.0 Hz, J=8.5 Hz, 1H), 7.08 (m, 2H), 4.17 (q, 2H), 1.49 (t, J=7.0 Hz, 3H).

### Intermediate 6

### 1-bromo-3-(chloromethyl)-5-ethoxybenzene

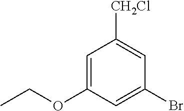

### Step A: Synthesis of ethyl 3-bromo-5-ethoxybenzoate

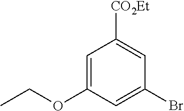

To a solution of 3-Bromo-5-hydroxybenzoic acid (3 g, 13.82 mmol) in DMF K₂CO₃ (5.73 g, 41.5 mmol) was added portion wise. To this, iodoethane (5.39 g, 34.6 mmol) was added slowly and the reaction mixture was stirred overnight at 50° C. It was diluted with ether:hexane(1:1)˜100 mL and 100 mL water. Layers were separated and the organic layer was washed with 100 ml brine, dried over sodium sulfate, filtered and concentrated to give the desired product as a white solid. ¹H-NMR (CDCl₃): [symbol] : 7.73 (s, 1H), 7.47 (s, 1H), 7.20 (bs, 1H), 4.36 (q, 2H), 4.05 (q, 2H), 1.40 (m, 6H).

### Intermediate 7

### 4,4-dimethyl-3,4-dihydro-2H-chromene-6-carbaldehyde

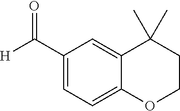

### Step A: Synthesis of 4-bromophenyl 3-methylbut-3-en-1-yl ether

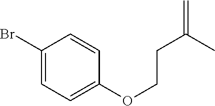

To a 250 ml round bottom flask 4-bromo phenol (4.08 g, 23.6 mmol), 3-methylbut-3-en-1-yl diphenyl phosphate (5.0 g, 15.7 mmol, synthesized according to a procedure in U.S. Pat. No. 5,006,550, 9 Apr. 1991), Cs₂CO₃ (15.4 g, 47.1 mmol) and 25 ml DMF were added. The resulting reaction mixture was heated at 130° C. for 15 min. The reaction mixture was cooled to room temperature and was diluted with 100 ml water and 100 ml EtOAc/hexane (1:2). Layers were separated. Organics were washed with 30 ml brine, dried over sodium sulfate, filtered and concentrated. Residue was purified on silica gel column, eluted with gradient solvent from 100% hexane to 1:9 E/H to give the desired product as a light yellow oil. ¹H-NMR (CDCl₃): [symbol] : 7.39 (d, J=8.8 Hz, 2H), 6.80 (d, J=8.8 Hz, 2H), 4.87 (s, 1H), 4.81 (s, 1H), 4.06 (t, J=6.9 Hz, 2H), 2.51 (t, J=6.9 Hz, 2H), 1.82 (s, 3H).

### Step B: Synthesis of 4-bromophenyl 3-methylbut-3-en-1-yl ether

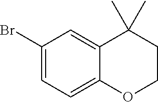

To a 100 ml round bottom flask AlCl₃ and 10 ml CH₂Cl₂ were added. A solution of 4-bromophenyl 3-methylbut-3-en-1-yl ether in 10 ml CH₂Cl₂ was added at 0° C. The resulting reaction mixture was stirred at 0° C. for 30 min. It was poured into an Erlenmeyer flask contains 100 ml 10% KOH and crushed ice. The resulting mixture was extracted with 75 ml hexane. Organics were dried over sodium sulfate, filtered and concentrated. Residue was purified on silica gel column, eluted with gradient solvent from 100% hexane to 1:9 E/H to give the desired product as a colorless oil. ¹H-NMR (CDCl₃): [symbol] : 7.37 (s, 1H), 7.18 (d, J=8.7 Hz, 1H), 6.70 (d, J=8.7 Hz, 1H), 4.21 (m, 2H), 1.85 (m, 2H), 1.36 (s, 6H).

### Step C: Synthesis of 4,4-dimethyl-3,4-dihydro-2H-chromene-6-carbaldehyde

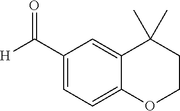

To a N₂ flushed 100 ml round bottom flask 4-bromophenyl 3-methylbut-3-en-1-yl ether (1 g, 4.15 mmol) and 10 ml THF were added. A solution of n-BuLi (1.76 ml, 2.6 M hexane solution) was added via a syringe at −78° C. The resulting reaction mixture was stirred at −78° C. for 10 minutes. DMF (0.48 ml, 6.22 mmol) was added. The reaction mixture was allowed to warm up to room temperature. EtOAc (25 ml) and wet silica gel (10 g silica gel/0.5 ml water) were added. The resulting mixture was stirred at room temperature for 10 minutes and filtered. The resulting solid was rinsed with EtOAc. The filtrate was concentrated and the residue was purified on Silica gel column, eluted with gradient solvent from 100% hexane to 1:4 E/H to give the desired product as a colorless oil. ¹H-NMR (CDCl₃): [symbol] : 9.87 (s, 1H), 7.85 (s, 1H), 7.62 (d, J=8.4 Hz, 1H), 6.91 (d, J=8.5 Hz, 1H), 4.30 (t, J=5.5 Hz, 2H), 1.89 (t, J=5.5 Hz, 2H), 1.40 (s, 6H).

### Example 1

### 8-[(2,6-diethoxy-4′-fluorobiphenyl-4-yl)methyl]-2,8-diazaspiro[4.5]decan-3-one

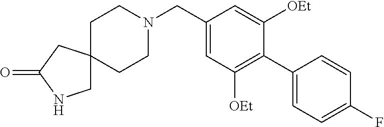

### Step A: Synthesis of 2,8-diazaspiro[4.5]decan-3-one hydrochloride

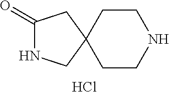

To a solution of tert-butyl 3-oxo-2,8-diazaspiro[4.5]decane-8-carboxylate (1.2 g, 4.7 mmol, Pharmaron) in 10 mL EtOAc was added HCl (23.7 ml, 2 M) in ether. The resulting reaction mixture was stirred at room temperature for 48 hours, then diluted with 60 mL hexane, filtered and air dried to give the title compound as light brown solid. ¹H-NMR (CD₃OD): [symbol] : 3.2 (b, 6H), 2.22 (s, 2H), 1.89 (b, 4H).

### Step B: Synthesis of 8-[(2,6-diethoxy-4′-fluorobiphenyl-4-yl)methyl]-2,8-diazaspiro[4.5]decan-3-one

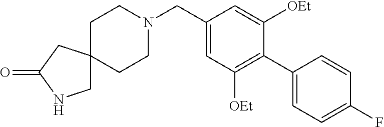

To a round bottom flask was added 2,8-diazaspiro[4.5]decan-3-one hydrochloride (0.50 g, 2.62 mmol); 4-(chloromethyl)-2,6-diethoxy-4′-fluorobiphenyl (0.81 g, 2.62 mmol); Cs₂CO₃ (2.1 g, 6.6 mmol); and 8 mL DMF. The resulting reaction mixture was stirred at 60° C. overnight. After cooling to room temperature, reaction mixture was diluted with 50 mL of EtOAc, washed with 60 mL of water, and 20 mL of brine. The organic layer was separated, dried over sodium sulfate, filtered and concentrated. The resulting residue was purified by silica gel chromatography by eluting with a gradient of 1:30 to 1:15 of 2M NH₃ in MeOH/CH₂Cl₂ to give the title compound. ¹H-NMR (CD₃OD): [symbol] : 7.28 (m, 2H), 7.05 (m, 2H), 6.69 (s, 2H), 3.97 (q, J=7 Hz, 4H), 3.53 (s, 2H), 3.22 (s, 2H), 2.5 (b, 4H), 2.24 (s, 2H), 1.73 (m, 4H), 1.22 (t, J=7 Hz, 6H). Mass Spectra (m/e): 427 (M+1).

### Example 2

### 4-{8-[(2,6-diethoxy-4′-fluorobiphenyl-4-yl)methyl]-3-oxo-2,8-diazaspiro[4.5]dec-2-yl}-benzoic acid

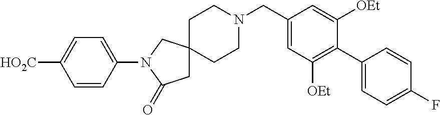

### Step A: Synthesis of Methyl 4-{8-[(2,6-diethoxy-4′-fluorobiphenyl-4-yl)methyl]-3-oxo-2,8-diazaspiro[4.5]dec-2-yl}-benzoate trifluoromethyl acetate

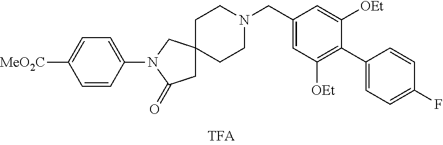

To a vial was added 8-[(2,6-diethoxy-4′-fluorobiphenyl-4-yl)methyl]-2,8-diazaspiro[4.5]decan-3-one (95 mg, 0.223 mmol, Example 1), methyl 4-bromobenzoate (72 mg, 0.334 mmol); Cs₂CO₃ (145 mg, 0.445 mmol); CuI (12.7 mg, 0.067 mmol); N,N-dimethylethane-1,2-diamine (14.7 mg, 0.167 mmol); and 1 mL dioxane. The reaction mixture was thoroughly degassed with nitrogen and heated at 110° C. overnight. After cooling to room temperature, the reaction mixture was diluted with 7 mL EtOAc, washed with 7 mL water and 1 mL NH₃ (saturated). The organic layer was separated, dried over sodium sulfate, filtered and concentrated. The resulting crude material was purified by HPLC with a reverse phase column by eluting with a gradient of 90/10 to 10/90 water/acetonitrile (containing 0.1% TFA) as the eluent to give the title compound as colorless solid. ¹H-NMR (CD₃OD): [symbol] : 8.04 (b, 2H), 7.8 (b, 2H), 7.28 (m, 2H), 7.09 (m, 2H), 6.84 (s, 2H), 4.35 (s, 2H), 4.0 (m, 4H), 3.9 (s, 3H), 3.2-3.6 (b, 4H), 2.2-2.6 (b, 2H), 1.9-2.2 (b, 4H), 1.25 (t, J=7 Hz, 6H). Mass Spectra (m/e): 561 (MA).

### Step B: Synthesis of 4-{8-[(2,6-diethoxy-4′-fluorobiphenyl-4-yl)methyl]-3-oxo-2,8-diazaspiro[4.5]dec-2-yl}benzoic acid trifluoromethyl acetate

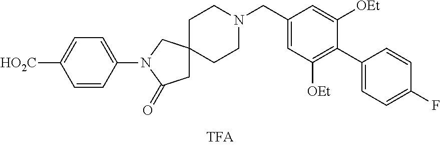

To a vial was added methyl 4-{8-[(2,6-diethoxy-4′-fluorobiphenyl-4-yl)methyl]-3-oxo-2,8-diazaspiro[4.5]dec-2-yl}benzoate trifluoromethyl acetic acid salt (95 mg, 0.141 mmol, from Step A); LiOH (20.2 mg, 0.85 mmol); 2 mL MeOH; and 0.2 mL water. The resulting reaction mixture was heated at 50° C. overnight, then the volatiles were removed. The resulting residue was acidified with TFA and purified by HPLC with a reverse phase column by eluting with a gradient of 90/10 to 10/90 of water/acetonitrile (containing 0.1% TFA) as the eluent to give the title compound. ¹H-NMR (CD₃OD): [symbol] : 8.05 (d, J=8.8 Hz, 2H), 7.77 (d, J=8.8 Hz, 2H), 7.27 (m, 2H), 7.08 (m, 2H), 6.84 (s, 2H), 4.3 (s, 2H), 4.03 (q, J=7.0 Hz, 4H), 3.9 (b, 2H), 3.3 (b, 4H), 2.7 (b, 2H), 2.04 (b, 4H), 1.25 (t, J=7 Hz, 6H). Mass Spectra (rule): 547 (M+1).

### Example 3

### 4-{8-[(2,6-diethoxy-4′-fluorobiphenyl-4-yl)methyl]-3-oxo-2,8-diazaspiro[4.5]dec-2-yl}benzamide trifluoromethyl acetate

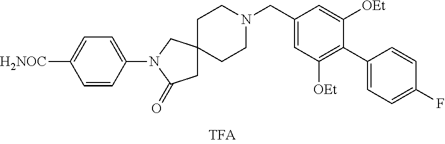

To a vial was added 4-{8-[(2,6-diethoxy-4′-fluorobiphenyl-4-yl)methyl]-3-oxo-2,8-diazaspiro[4.5]dec-2-yl}benzoic acid trifluoromethyl acetic acid salt (46 mg, 70 mmol, Example 2); BOP reagent (37 mg, 84 mmol); and 1 mL THF. The resulting reaction mixture was stirred at room temperature for 15 minutes. Then ammonia gas was bubbled into the reaction mixture for 1 minute. The reaction mixture concentrated, and the resulting residue was acidified with TFA and purified by HPLC with a reverse phase column by eluting with a gradient of 90/10 to 10/90 of water/acetonitrile (containing 0.1% TFA) as the eluent to give the title compound as fluffy white solid after lyophilizing from CH₃CN/water. ¹H-NMR (CD₃OD): [symbol] : 7.9 (m, 2H), 7.75 (m, 2H), 7.27 (m, 2H), 7.08 (m, 2H), 6.82 (m, 2H), 4.34 (s, 2H), 4.03 (m, 4H), 3.8 (s, 2H), 3.2-3.6 (m, 4H), 2.6-2.8 (m, 2H), 1.9-2.2 (m, 4H), 1.25 (m, 6H). Spectrum is complicate and consists of a pair of rotomers. MS (m/e): 546 (M+1).

### Example 4

### Methyl 4-{8-[3,5-bis(cyclopropyloxy)-4-(3,5-difluoropyridin-2-yl)benzyl]-3-oxo-2,8-diazaspiro[4.5]dec-2-yl}benzoate trifluoromethyl acetic acid salt

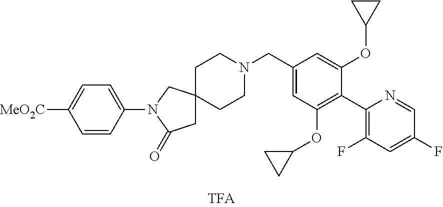

To a vial was added methyl 4-(3-oxo-2,8-diazaspiro[4.5]dec-2-yl)benzoate trifluoromethyl acetate (58 mg, 0.145 mmol, Intermediate 2); 3,5-bis(cyclopropyloxy)-4-(3,5-difluoropyridin-2-yl)benzaldehyde (40 mg, 0.121 mmol, Intermediate 3); sodium triacetoxyborohydride (77 mg, 0.362 mmol); TEA (0.05 ml, 0.36 mmol); AcOH (0.048 ml, 0.85 mmol); and 2 mL THF. The resulting reaction mixture was heated at 30° C. overnight, and the volatiles were removed. The resulting residue was acidified with TFA, and purified by HPLC with a reverse phase column by eluting with a gradient of 90/10 to 10/90 of water/acetonitrile (containing 0.1% TFA) as the eluent to give the title compound. ¹H-NMR (CD₃OD): [symbol] : 8.4 (s, 1H) 8.05 (m, 2H), 7.8 (m, 2H), 7.65 (m, 1H), 7.25 (m, 2H), 4.44 (s, 2H), 3.2-4.0 (m, 8H), 2.6-2.8 (m, 2H), 1.9-2.2 (m, 4H), 1.6-1.8 (m, 8H). Spectrum is complicate and consists of a pair of rotomers.

Mass Spectra (m/e): 590 (M+1).

### Example 5

### 4-{8-[(2,6-diethoxy-4-bromophenyl-4-yl)methyl]-3-oxo-2,8-diazaspiro[4.5]dec-2-yl}benzoic acid trifluoromethyl acetate

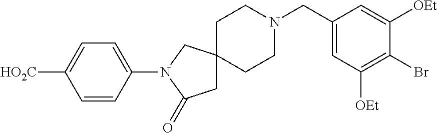

### Step A: Synthesis of methyl 4-{8-[(2,6-diethoxy-4-bromophenyl-4-yl)methyl]-3-oxo-2,8-diazaspiro[4.5]dec-2-yl}benzoate

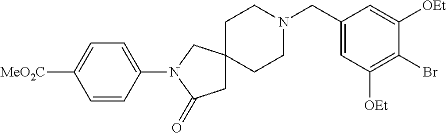

To a solution of methyl 4-(3-oxo-2,8-diazaspiro[4.5]dec-2-yl)benzoate hydrochloride (0.85 g, 2.95 mmol, Intermediate 3) in DMF (20 mL) was added DIPEA (1.52 gm, 11.79 mmol), followed by 4-bromo-3,5-diethoxybenzyl bromide (1.21 g, 4.13 mmol). The reaction mixture was heated to 60° C. and then stirred for 2 hours. The reaction mixture was then diluted with 50 mL EtOAc and 50 mL 10% KOH, and was stirred for 2 minutes. The resulting layers were separated, and the water layer was extracted with 50 mL of EtOAc. The organic layers were combined, washed with 50 mL brine, dried over sodium sulfate, filtered and concentrated. The resulting residue was purified on a CombiFlash™ column by eluting with 1:1 ethyl acetate/hexane, then with 100% ethyl acetate, and then with 10% MeOH in DCM to give the title compound as white solid. ¹H-NMR (CDCl₃): [symbol] : 8.06 (d, J=9.0 Hz, 2H), 7.73 (d, J=8.0 Hz, 2H), 6.56 (s, 2H), 4.12 (q, 4H), 3.9 (s, 3H), 3.69 (s, 2H), 3.47 (s, 2H), 2.46 (m, 6H), 1.76 (m, 4H), 1.48 (t, 6H). Mass Spectra (m/e): 545 (M+1).

### Step B: Synthesis of 4-{8-[(2,6-diethoxy-4-bromophenyl-4-yl)methyl]-3-oxo-2,8-diazaspiro[4.5]dec-2-yl}benzoic acid trifluoromethyl acetate

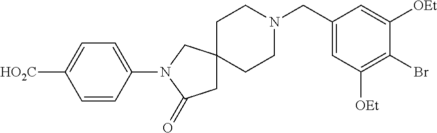

To a solution of methyl 4-{8-[(2,6-diethoxy-4-bromophenyl-4-yl)methyl]-3-oxo-2,8-diazaspiro[4.5]dec-2-yl}benzoate (Step A, 1.3 g, 2.38 mmol), in methanol (10 mL) and water (2 mL), was added KOH (0.70 g, 11.9 mmol). The resulting reaction mixture was heated at 60° C. for 2 hours, then the volatiles were removed. Water (10 ml) was added to the resulting residue, and pH was adjusted to 6.5 with 1N HCl. The resulting solid was collected by filtration and was air dried to give the title compound as white solid. ¹H-NMR (CD₃OD); [symbol] : 8.04 (d, J=9.0 Hz, 2H), 7.76 (d, J=8.5 Hz, 2H), 6.81 (s, 2H), 426 (s, 2H), 4.17 (q, 4H), 3.89 (bs, 2H), 3.31 (m, 4H, merged with solvent peak), 2.69 (bs, 2H), 2.02 (bs, 4H), 1.46 (t, 6H). Mass Spectra (m/e): 531 (M+1).

### Example 6

### 8-[(2,6-diethoxy-4′-fluorobiphenyl-4-yl)methyl]-2-[4-(5-oxo-4,5-dihydro-1,2,4-oxadiazol-3-yl)phenyl]-2,8-diazaspiro[4.5]decan-3-one

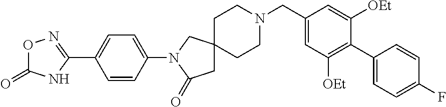

### Step A: Synthesis of 4-{8-[(2,6-diethoxy-4′-fluorobiphenyl-4-yl)methyl]-3-oxo-2,8-diazaspiro[4.5]dec-2-yl}benzonitrile

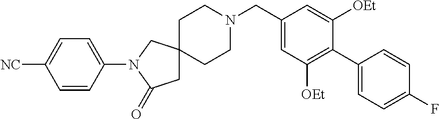

To a vial was added 8-[(2,6-diethoxy-4′-fluorobiphenyl-4-yl)methyl]-2,8-diazaspiro[4.5]decan-3-one (150 mg, 035 mmol, Intermediate 3), 4-bromobenzonitrile (128 mg, 0.70 mmol), K₃PO₄ (224 mg, 1.05 mmol), Pd₂(dba)₃ (9.7 mg, 10.6 μmol), 9,9-dimethyl-4,5-bis(diphenylphosphino)-xanthene (10.2, 10.6 μmol) and 1.5 mL dioxane. The reaction mixture was thoroughly degassed with nitrogen, and heated at 110° C. overnight. After cooling to room temperature, the reaction mixture was diluted with 10 mL EtOAc, and washed with 10 mL water. The organic layer was separated, dried over sodium sulfate, filtered and concentrated. The resulting crude material was purified via silica gel chromatography by eluting with 1:1 ethylacetate/hexane, followed by 10% MeOH in DCM to give the title compound. ¹H-NMR (CD₃OD): [symbol] : 7.88 (d, J=8.9 Hz, 2H), 7.74 (d, J=8.8 Hz, 2H), 7.26 (m, 2H), 7.04 (m, 2H), 6.71 (s, 2H), 3.97 (q, J=7.0 Hz, 4H), 3.79 (s, 2H), 3.57 (s, 2H), 2.62 (b, 2H), 2.59 (s, 2H), 2.5 (b, 2H), 1.8 (b, 4H), 1.22 (t, J=7.0 Hz, 6H).

Mass Spectra (m/e): 528 (M+1).

### Step B: Synthesis of 8-[(2,6-diethoxy-4′-fluorobiphenyl-4-yl)methyl]-2-[4-(5-oxo-4,5-dihydro-1,2,4-oxadiazol-3-yl)phenyl]-2,8-diazaspiro[4.5]decan-3-one

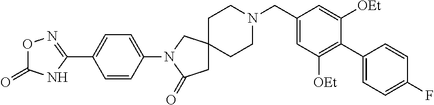

To a round bottom flask was added 4-{8-[(2,6-diethoxy-4′-fluorobiphenyl-4-yl)methyl]-3-oxo-2,8-diazaspiro[4.5]dec-2-yl}benzonitrile (Step A, 30 mg, 0.057 mmol), hydroxylamine (37 mg, 50% water solution, 0.57 mmol) and 2 mL EtOH. The reaction mixture was heated at 80° C. for 1 hour, then the volatiles were removed. The resulting residue was dissolved in 0.5 mL dioxane, then phosgene (0.89 ml, 1 M toluene solution) was added via syringe. The reaction mixture was heated at 80° C. for 1 hour, followed by removal of the volatiles. The resulting residue was acidified with TFA and purified by HPLC with a reverse phase column by eluting with a gradient of 90/10 to 10/90 of water/acetonitrile (containing 0.1% TFA) as the eluent to give title compound as fluffy white solid after lyophilizing from CH₃CN/water. ¹H-NMR (CD₃OD): [symbol] : 7.87 (d, J=8.9 Hz, 2H), 7.83 (d, J=8.8 Hz, 2H), 7.27 (m, 2H), 7.08 (m, 2H), 6.84 (s, 2H), 4.32 (s, 2H), 4.03 (q, J=7.0 Hz, 4H), 3.9 (b, 2H), 3.3 (b, 4H, overlapping with solvent peak), 2.7 (b, 2H), 2.05 (b, 4H), 1.25 (t, J=7.0 Hz, 6H). Mass Spectra (m/e): 587 (M+1).

### Example 7

### 8-[(2,6-diethoxy-4′-fluorobiphenyl-4-yl)methyl]-2-[4-(1-H-tetrazol-5-yl)phenyl]-2,8-diazaspiro[4.5]decan-3-one trifluoromethyl acetate

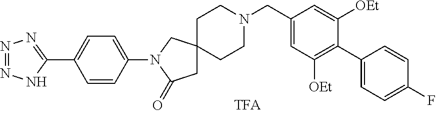

To a round bottom flask was added 4-{8-[(2,6-diethoxy-4′-fluorobiphenyl-4-yl)methyl]-3-oxo-2,8-diazaspiro[4.5]dec-2-yl}benzonitrile (60 mg, 0.094 mmol, Example 6), TMSN₃ (58 mg, 0.28 mmol) and 2 mL toluene. The reaction mixture was heated to reflux overnight, followed by removal of the volatiles. The resulting residue was acidified with TFA and purified by HPLC with a reverse phase column by eluting with a gradient of 90/10 to 10/90 of water/acetonitrile (containing 0.1% TFA) as the eluent to give the title compound as a fluffy white solid after lyophilizing from CH₃CN/water. ¹H-NMR (CD₃OD): [symbol] : 8.05 (b, 2H), 7.9 (b, 2H), 7.27 (m, 2H), 7.07 (m, 2H), 6.84 (s, 2H), 4.35 (s, 2H), 4.03 (q, J=7.0 Hz, 4H), 3.85 (b, 2H), 3.55 (b, 3H), 3.25 (b, 2H), 2.6-2.8 (b, 2H), 1.9-2.2 (b, 4H), 1.25 (t, J=7.0 Hz, 6H). Mass Spectra (m/e): 571 (M+1).

### Example 8

### {8-[(2,6-diethoxy-4′-fluorobiphenyl-4-yl)methyl]-3-oxo-2,8-diazaspiro[4.5]dec-2-yl}acetic acid trifluoromethyl acetate

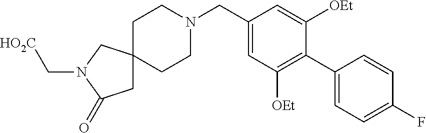

### Step A: Synthesis of tert-butyl-2-(2-methoxy-2-oxoethyl)-3-oxo-2,8-diazaspiro[4.5]decane-8-carboxylate

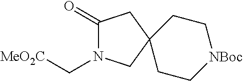

To a nitrogen flushed vial was added tert-butyl-3-oxo-2,8-diazaspiro[4.5]decane-8-carboxylate (240 mg, 0.944 mmol, Pharmaron), NaH (113 mg, 60%, 2.83 mmol), and 3 mL DMF. The reaction mixture was stirred at room temperature for 1 hour. Then methyl bromoacetate (433 mg, 2.83 mmol) was added at 0° C., and the resulting reaction mixture was stirred at 0° C. for 1 hour. The reaction was quenched with water, and extracted with EtOAc. The organic layer was separated, dried over sodium sulfate, filtered and concentrated. The resulting crude product was used for the next step of reaction without further purification. Mass Spectra (m/e): 327 (M+1).

### Step B: Synthesis of methyl (3-oxo-2,8-diazaspiro[4.5]dec-2-yl)acetate trifluoromethyl acetic acid salt

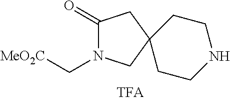

To a vial was added tert-butyl-2-(2-methoxy-2-oxoethyl)-3-oxo-2,8-diazaspiro-[4.5]decane-8-carboxylate (Step A, 300 mg, 0.919 mmol), TFA (0.35 ml, 4.6 mmol) and 3 mL CH₂Cl₂. The reaction was stirred at room temperature for 30 minutes, followed by removal of the volatiles. The crude product was used for the next step of reaction directly without further purification.

Mass Spectra (nee): 227 (M+1).

### Step C: Synthesis of Methyl{8-[(2,6-diethoxy-4′-fluorobiphenyl-4-yl)methyl]-3-oxo-2,8-diazaspiro[4.5]dec-2-yl}acetate trifluoromethyl acetic acid salt

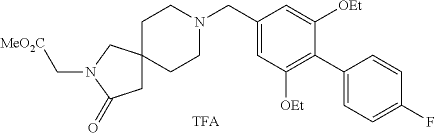

To a vial was added methyl (3-oxo-2,8-diazaspiro[4.5]dec-2-yl)acetate trifluoromethyl acetic acid salt (Step B, 200 mg, from the previous step), 4-(chloromethyl)-2,6-diethoxy-4′-fluorobiphenyl (328 mg, 1.06 mmol), DIEA (0.31 ml, 1.8 mmol) and 2 mL DMF. The reaction was heated at 50° C. overnight. The crude mixture was acidified with TFA, and purified by HPLC with a reverse phase column by eluting with a gradient of 90/10 to 10/90 of water/acetonitrile (containing 0.1% TFA) as the eluent to give the title compound.

¹H-NMR (CDCl₃): [symbol] : 7.29 (m, 2H), 7.09 (t, J=7.0 Hz, 2H), 6.83 (s, 2H), 4.32 (s, 2H), 4.11 (s, 3H), 4.05 (q, J=7 Hz, 4H), 3.40-3.60 (b, 2H), 3.33-3.40 (b, 4H), 2.20-2.60 (b, 2H), 1.85-2.20 (b, 4H) 1.23 (t, J=7.0 Hz, 6H). Mass Spectra (m/e): 499 (M+1).

### Step D: Synthesis of {8-[(2,6-diethoxy-4′-fluorobiphenyl-4-yl)methyl]-3-oxo-2,8-diazaspiro[4.5]dec-2-yl}acetic acid trifluoromethyl acetic acid salt

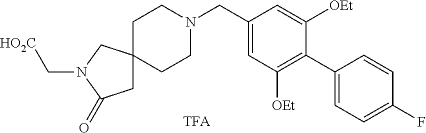

To a vial was added methyl {8-[(2,6-diethoxy-4′-fluorobiphenyl-4-yl)methyl]-3-oxo-2,8-diazaspiro[4.5]dec-2-yl}acetate trifluoromethyl acetic acid salt (Step C, 60 mg, 0.12 mmol); LiOH (11.5 mg, 0.481 mmol); 1 mL water; and 1 mL MeOH. The reaction was heated at 50° C. for 1 hour, followed by removal of the volatiles. The resulting residue was acidified with TFA and purified by HPLC with a reverse phase column by eluting with a gradient of 90/10 to 10/90 of water/acetonitrile (containing 0.1% TFA) as the eluent to give the title compound. ¹H-NMR (CDCl₃): [symbol] : 7.29 (m, 2H), 7.09 (t, J=7.0 Hz, 2H), 6.83 (s, 2H), 4.32 (s, 2H), 4.05 (q, J=7 Hz, 4H), 3.40-3.60 (b, 2H), 3.33-3.40 (b, 4H), 2.20-2.60 (b, 2H), 1.85-2.20 (b, 4H) 1.23 (t, J=7.0 Hz, 6H). Mass Spectra (m/e): 485 (M+1).

### Example 9

### 8-[(2,6-diethoxy-4′-fluorobiphenyl-4-yl)methyl]-2-(2-hydroxyethyl)-2,8-diazaspiro[4.5]decan-3-one trifluoromethyl acetate

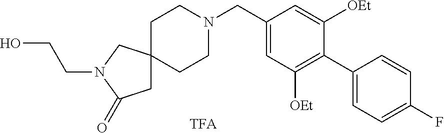

To a vial was added {8-[(2,6-diethoxy-4′-fluorobiphenyl-4-yl)methyl]-3-oxo-2,8-diazaspiro[4.5]dec-2-yl}acetic acid trifluoromethyl acetic acid salt (11 mg, 0.020 mmol, Example 8), BOP reagent (50 mg, 0.114 mmol), DIEA (0.02 ml, 0.114 mmol) and 2 mL DMF. The resulting reaction mixture was stirred at room temperature for 15 minutes, then NaBH₄ (4.3 mg, 0.114 mmol) was added. The reaction mixture was stirred at room temperature 30 minutes, then acidified with TFA, and purified by HPLC with a reverse phase column by eluting with a gradient of 90/10 to 10/90 of water/acetonitrile (containing 0.1% TFA) as the eluent to give the title compound. ¹H-NMR (CDCl₃): [symbol] : 7.29 (m, 2H), 7.09 (m, 2H), 6.83 (s, 2H), 4.32 (s, 2H), 3.90-4.05 (m, 6H), 3.60-3.75 (b, 2H), 3.15-3.20 (b, 2H), 3.33-3.40 (b, 4H), 2.20-2.60 (b, 2H), 1.85-2.20 (b, 4H) 1.23 (t, J=7.0 Hz, 6H). Mass Spectra (m/e): 471 (M+1).

### Example 10

### 4-{8-[4-(2,4-difluorophenyl)-3,5-diethoxybenzyl]-3-oxo-2,8-diazaspiro[4.5]dec-2-yl}benzoic acid trifluoromethyl acetate

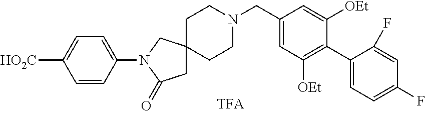

To a stirred solution of 4-{8-[(2,6-diethoxy-4-bromophenyl-4-yl)methyl]-3-oxo-2,8-diazaspiro[4.5]dec-2-yl}benzoic acid (Example 5, Step B, 25 mg, 0.047 mmol) in ethanol in a PYREX® VISTA™ culture tube was added K₃PO₄ (30 mg, 0.141 mmol), Pd (OAc)₂ (1.06 mg, 4.70 μmol), DTBPF (2.32 mg, 4.70 μmol), and then 2,4-difluorophenylboronic acid (14.86 mg 0.094 mmol). The reaction mixture was degassed with nitrogen, sealed and heated at 70° C. for 2 hours. The reaction mixture was filtered and then purified by reverse phase HPLC using an acetonitrile/water gradient to give the title compound as white solid. ¹H-NMR (CD₃OD): [symbol] : 7.9 (d, J=9.0 Hz, 2H), 7.5 (d, J=10.0 Hz, 2H), 7.1 (m, 1H), 6.7 (m, 2H), 6.5 (s, 2H), 3.8 (q, 4H, merged with solvent peak), 3.6 (s, 2H), 3.4 (s, 2H), 2.4 (m, 6H), 1.7 (m, 4H), 1.1 (t, 6H). Mass Spectra (m/e): 565 (M+1).

The Examples shown in Table 1 were prepared from the appropriate starting materials according to the methods described in Examples 1-9.

TABLE 1

| | | LC-Mass |
|---|---|---|
| Example | Compound | (M + 1) |
| 11 | 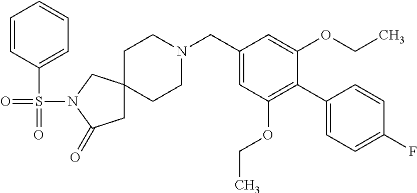 | 567 |
| 12 | 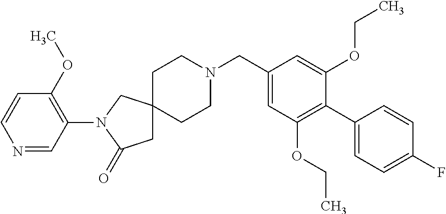 | 534 |
| 13 | 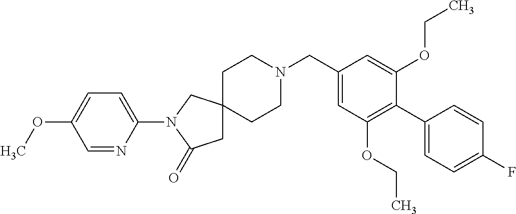 | 534 |
| 14 | 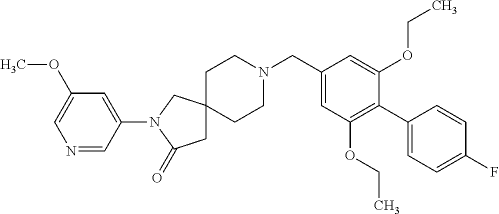 | 534 |
| 15 | 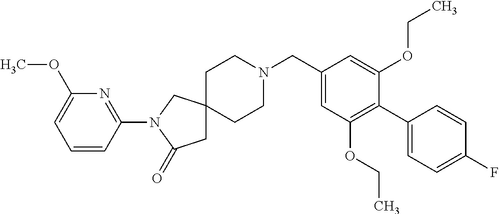 | 534 |
| 16 | 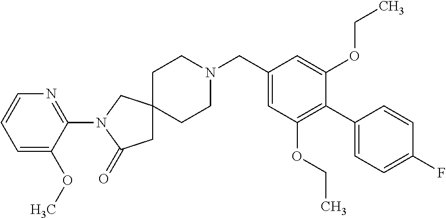 | 534 |
| 17 | 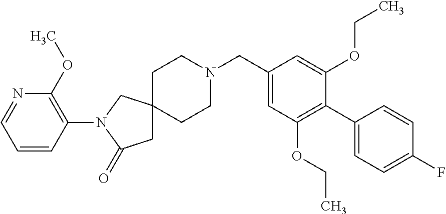 | 534 |
| 18 | 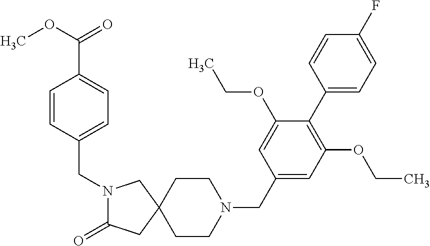 | 575 |
| 19 | 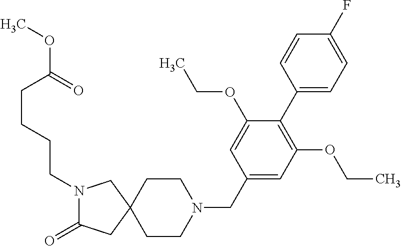 | 541 |
| 20 | 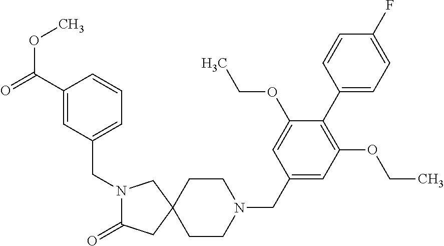 | 575 |
| 21 | 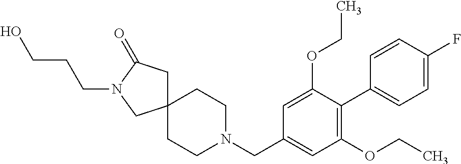 | 485 |
| 22 | 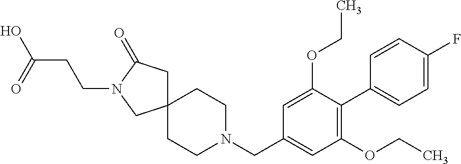 | 499 |
| 23 | 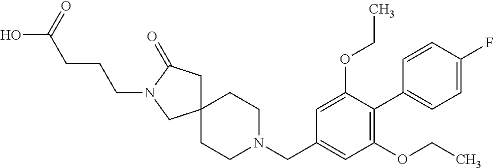 | 513 |
| 24 | 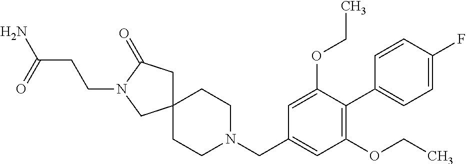 | 498 |
| 25 | 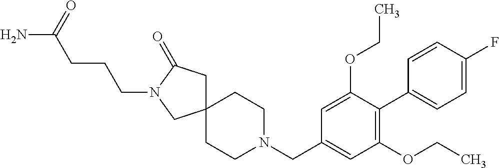 | 512 |
| 26 | 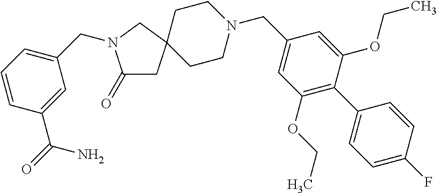 | 560 |
| 27 | 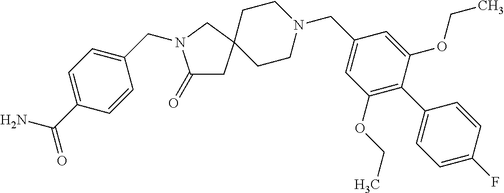 | 560 |
| 28 | 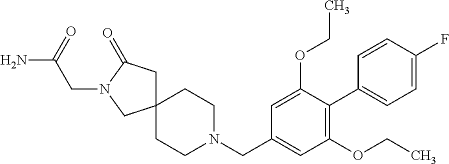 | 484 |
| 29 | 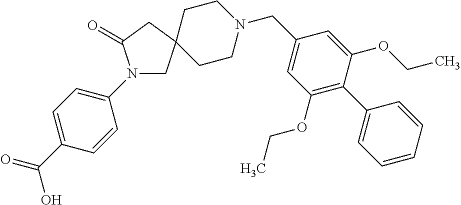 | 529 |
| 30 | 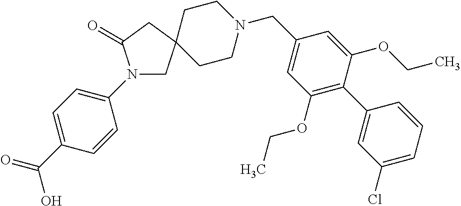 | 564 |
| 31 | 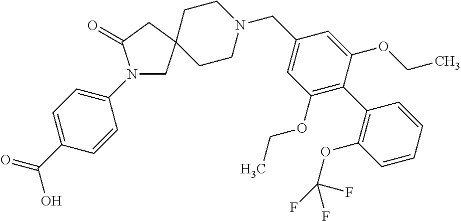 | 613 |
| 32 | 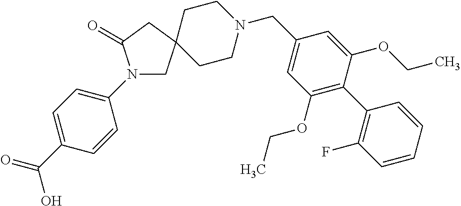 | 547 |
| 33 | 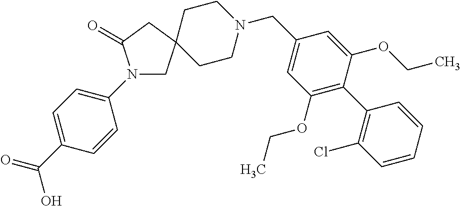 | 564 |
| 34 | 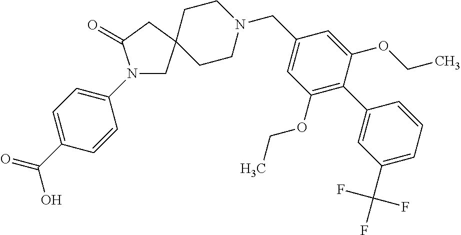 | 597 |
| 35 | 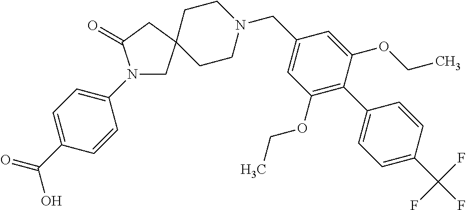 | 597 |
| 36 | 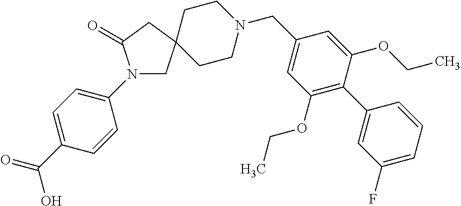 | 547 |
| 37 | 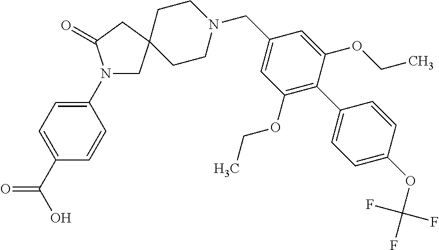 | 613 |
| 38 | 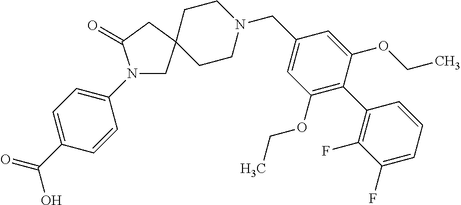 | 565 |
| 39 | 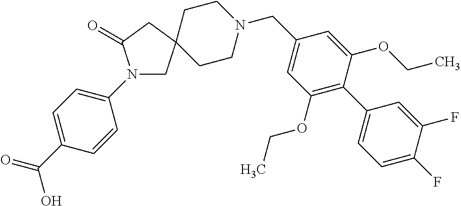 | 565 |
| 40 | 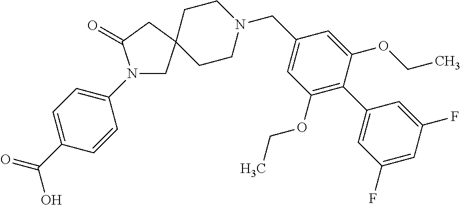 | 565 |
| 41 | 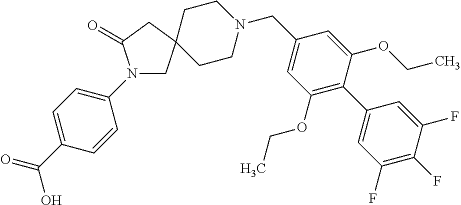 | 583 |
| 42 | 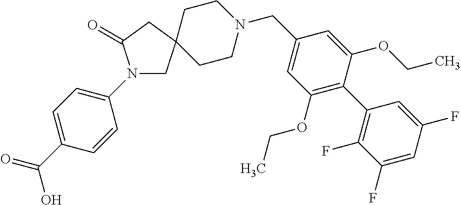 | 583 |
| 43 | 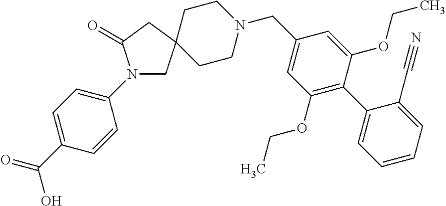 | 554 |
| 44 | 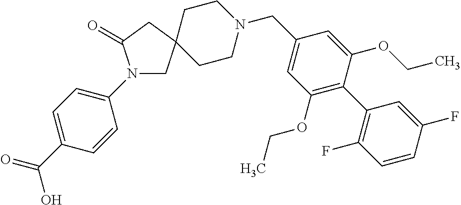 | 565 |
| 45 | 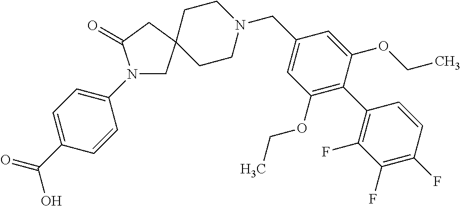 | 583 |
| 46 | 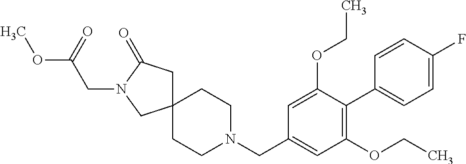 | 499 |
| 47 | 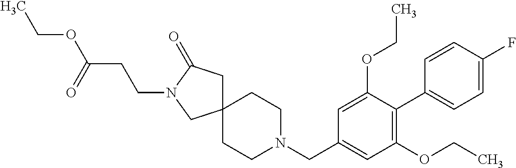 | 527 |
| 48 | 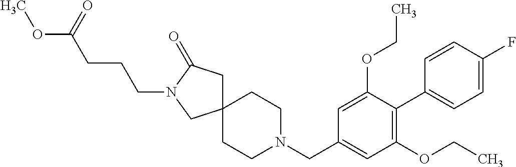 | 527 |
| 49 | 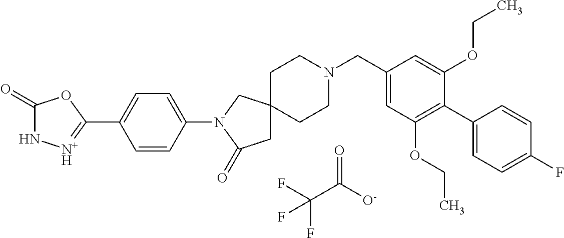 | 587 |
| 50 | 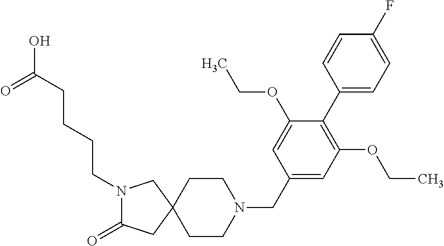 | 527 |
| 51 | 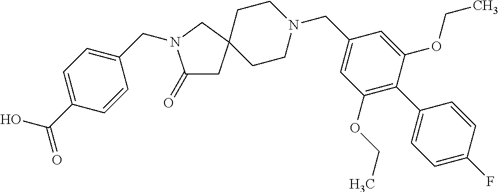 | 561 |
| 52 | 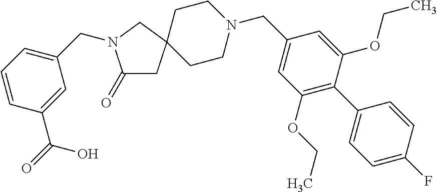 | 561 |
| 53 | 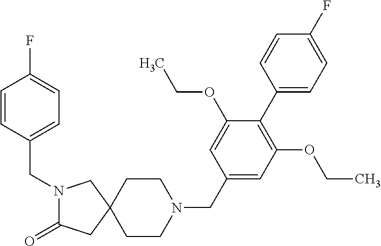 | 535 |
| 54 | 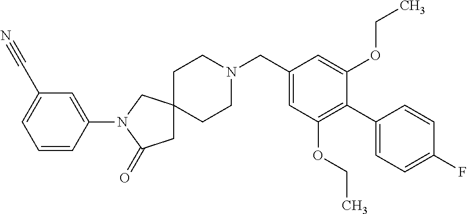 | 528 |
| 55 | 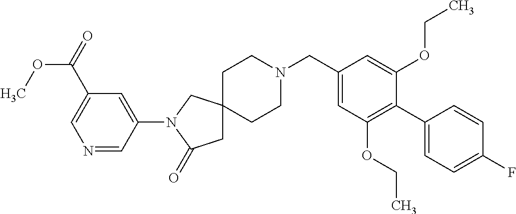 | 562 |
| 56 | 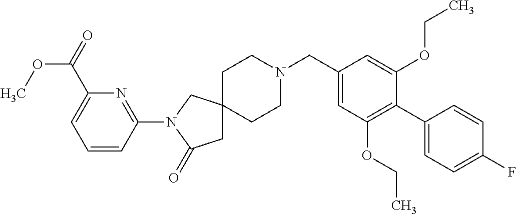 | 562 |
| 57 | 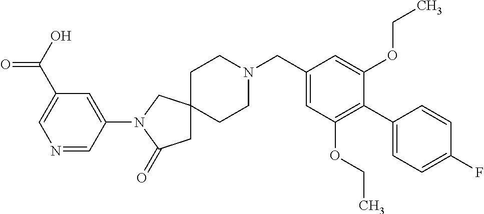 | 548 |
| 58 | 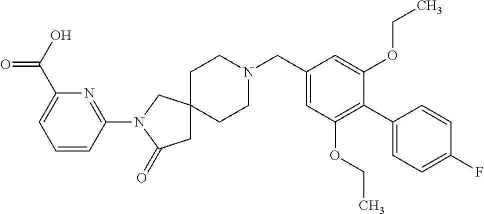 | 548 |
| 59 | 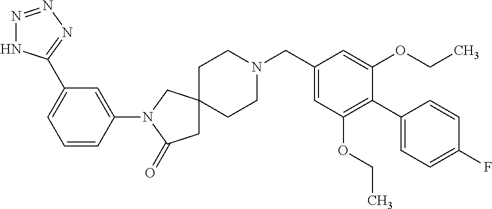 | 571 |
| 60 | 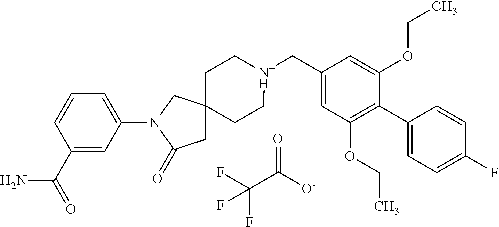 | 546 |
| 61 | 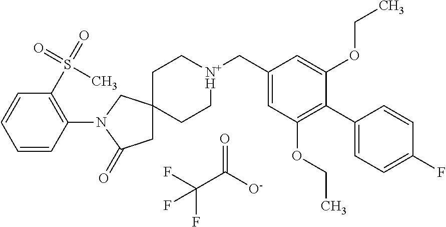 | 581 |
| 62 | 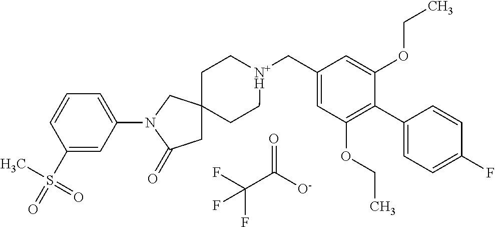 | 581 |
| 63 | 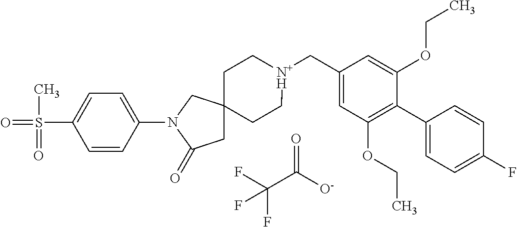 | 581 |
| 64 | 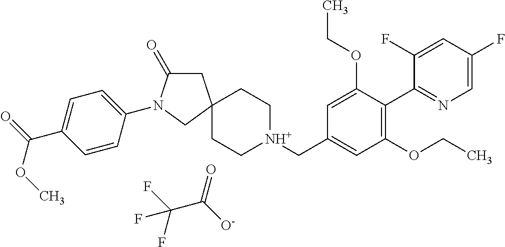 | 580 |
| 65 | 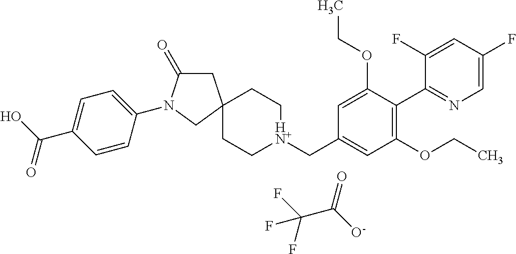 | 566 |
| 66 | 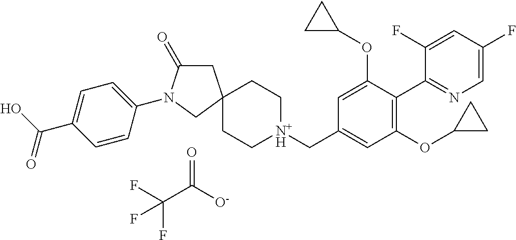 | 590 |
| 67 | 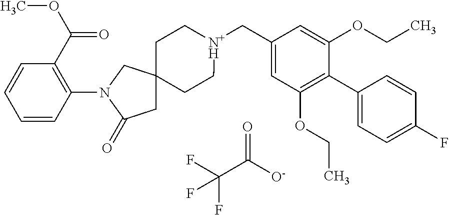 | 561 |
| 68 | 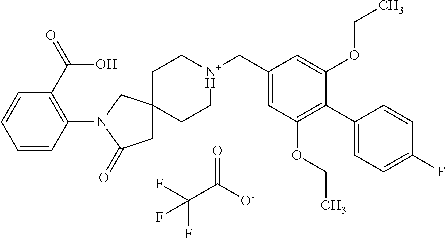 | 547 |
| 69 | 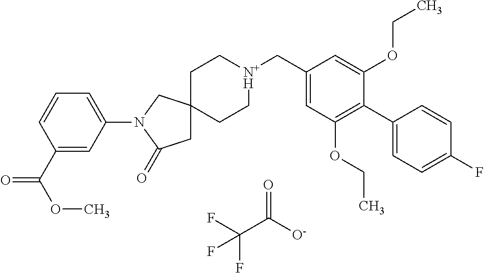 | 561 |
| 70 | 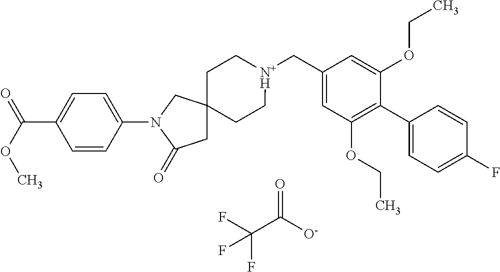 | 561 |
| 71 | 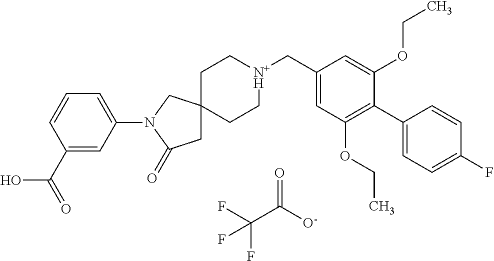 | 547 |
| 72 | 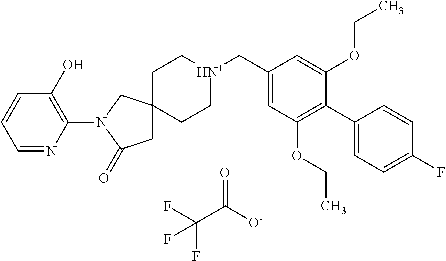 | 520 |
| 73 | 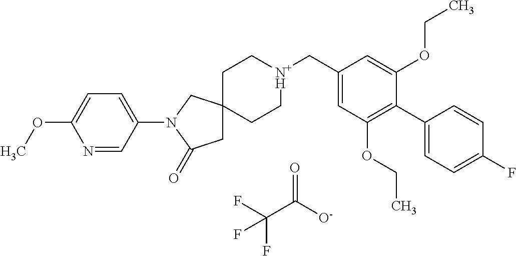 | 534 |
| 74 | 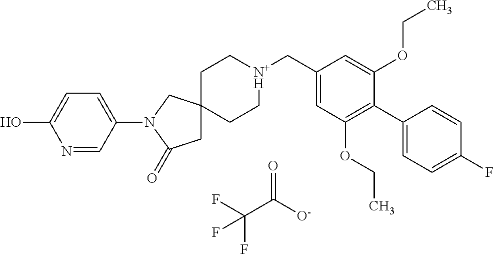 | 520 |
| 75 | 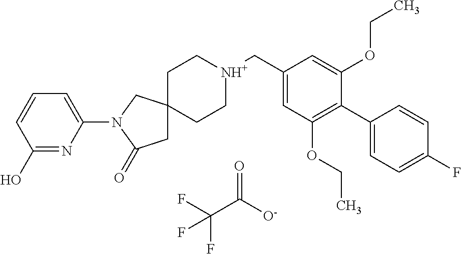 | 520 |
| 76 | 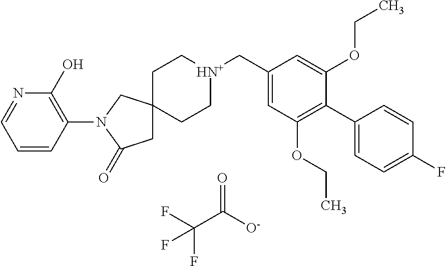 | 520 |
| 77 | 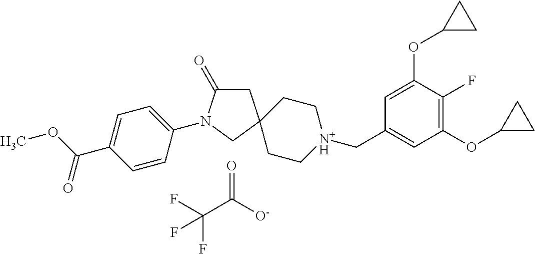 | 509 |
| 78 | 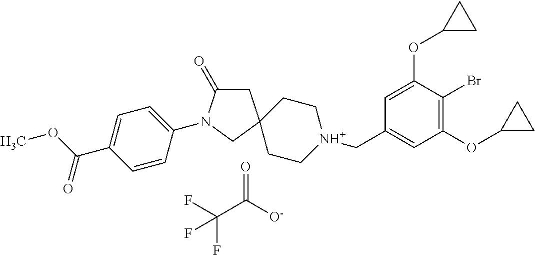 | 570 |
| 79 | 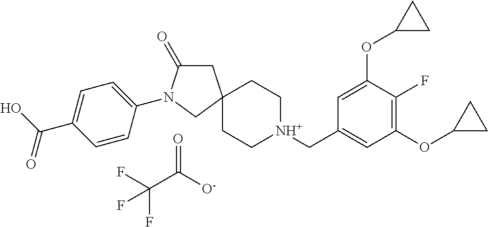 | 495 |
| 80 | 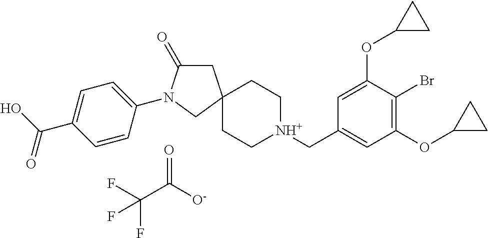 | 556 |
| 81 | 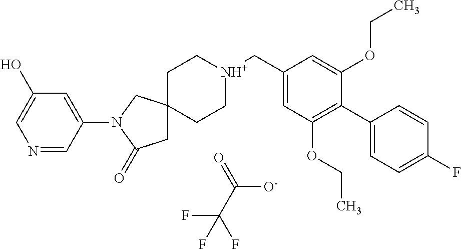 | 520 |
| 82 | 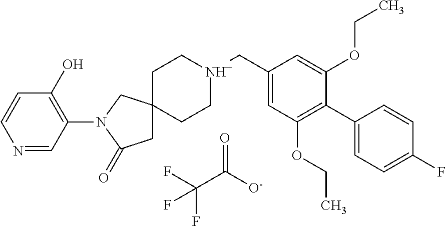 | 521 |
| 83 | 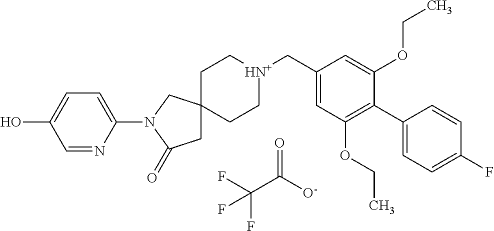 | 520 |
| 84 | 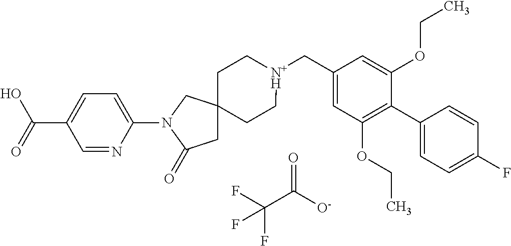 | 548 |
| 85 | 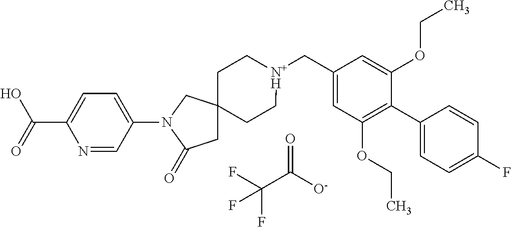 | 548 |
| 86 | 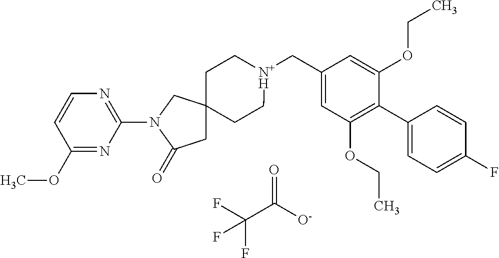 | 535 |
| 87 | 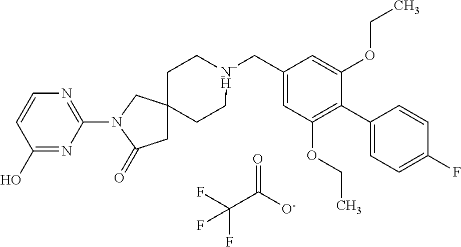 | 521 |
| 88 | 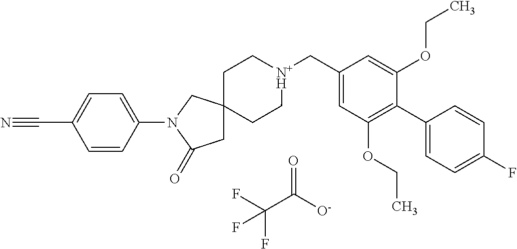 | 528 |
| 89 | 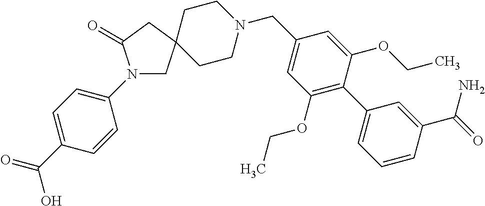 | 572 |
| 90 | 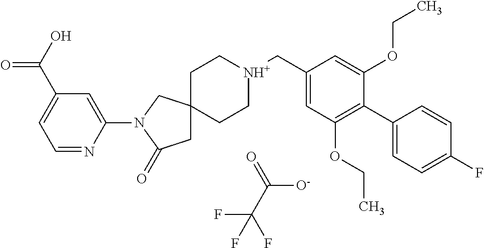 | 548 |
| 91 | 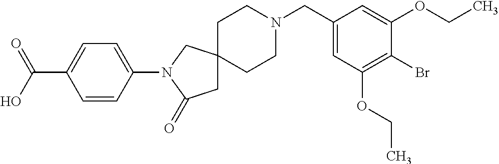 | 532 |
| 92 | 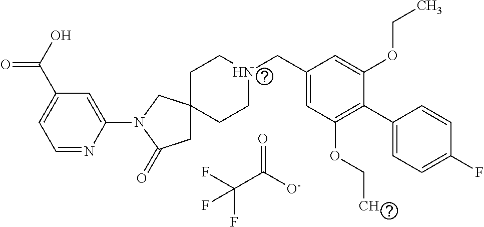 | 547 |
| 93 | 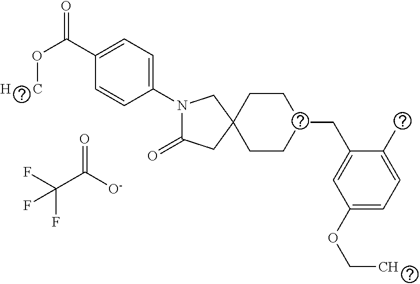 | 501 |
| 94 | 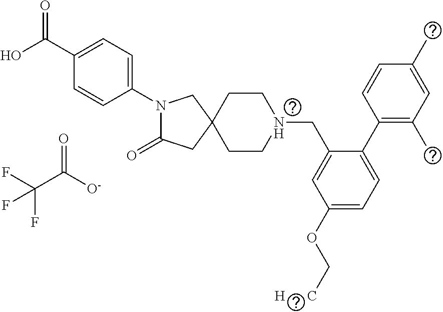 | 520 |
| 95 | 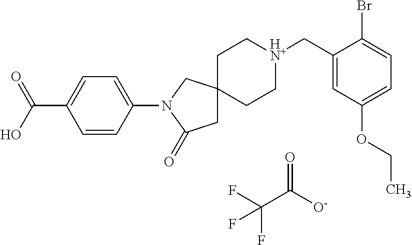 | 488 |
| 96 | 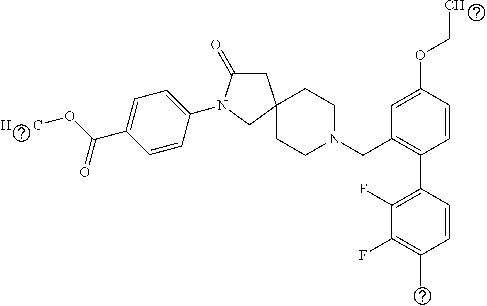 | 552 |
| 97 | 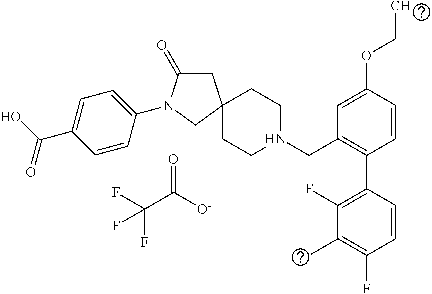 | 538 |
| 98 | 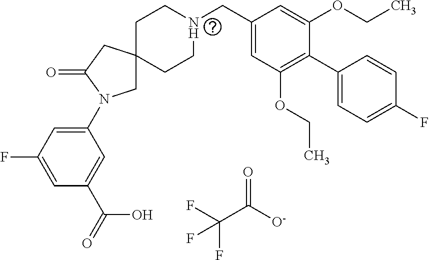 | 564 |
| 99 | 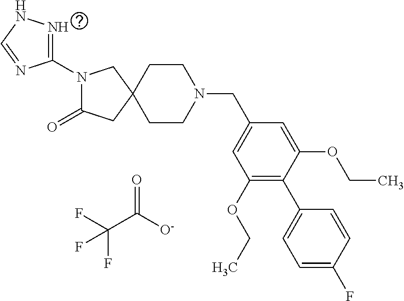 | 493 |
| 100 | 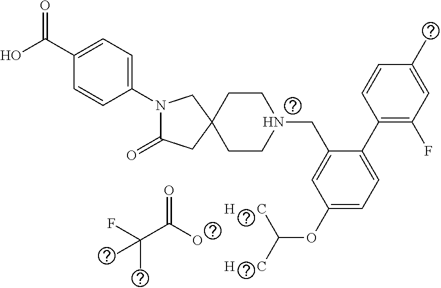 | 534 |
| 101 | 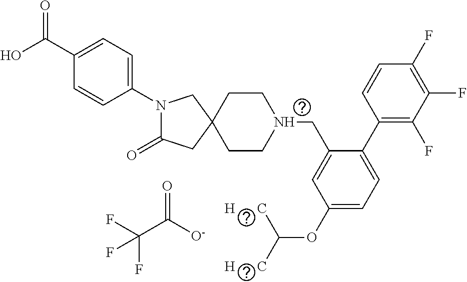 | 552 |
| 102 | 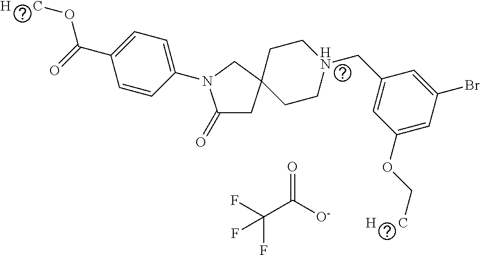 | 501 |
| 103 | 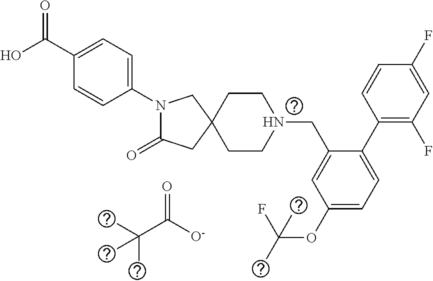 | 560 |
| 104 | 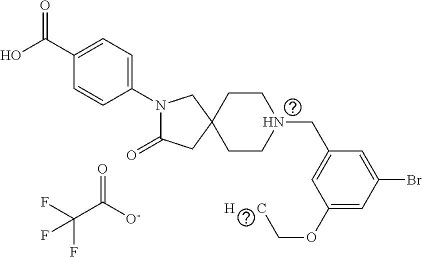 | 487 |
| 105 | 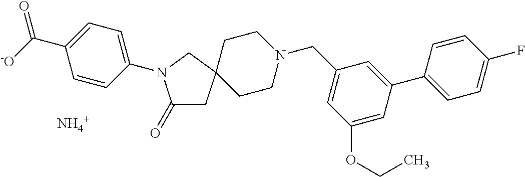 | 502 |
| 106 | 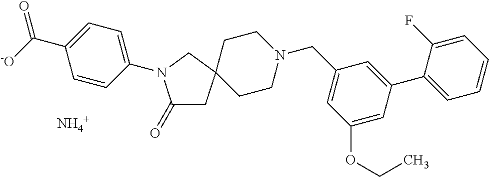 | 502 |
| 107 | 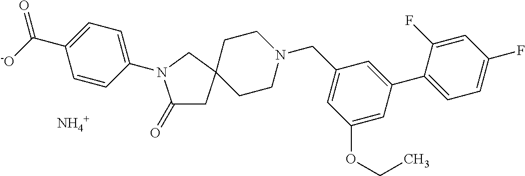 | 520 |
| 108 | 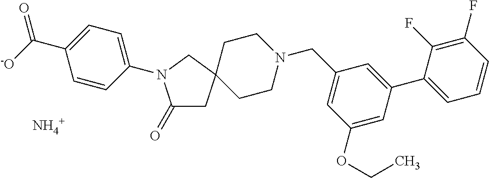 | 520 |
| 109 | 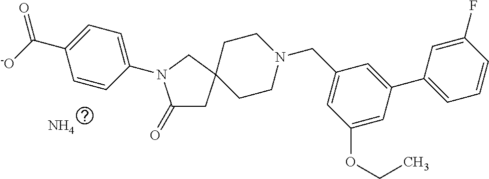 | 502 |
| 110 | 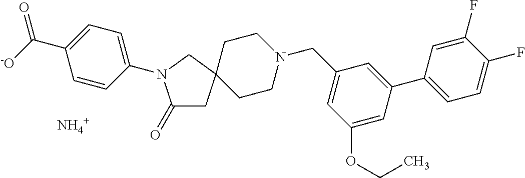 | 520 |
| 111 | 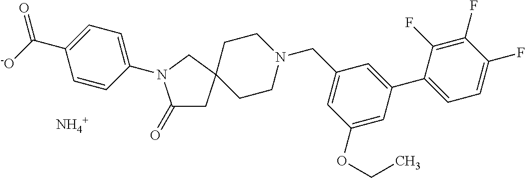 | 538 |
| 112 | 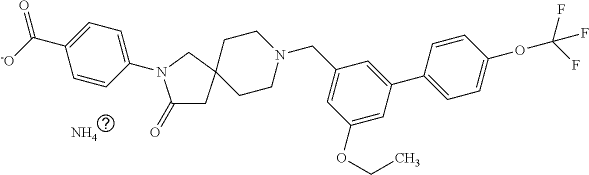 | 568 |
| 113 | 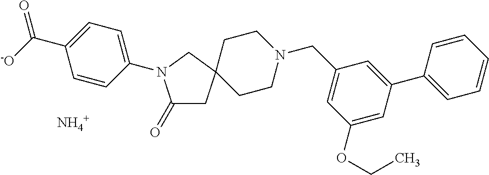 | 484 |
| 114 | 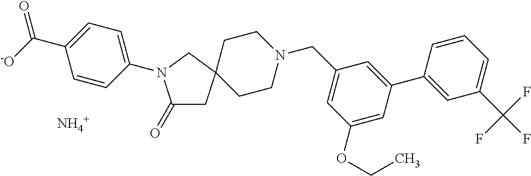 | 552 |
| 115 | 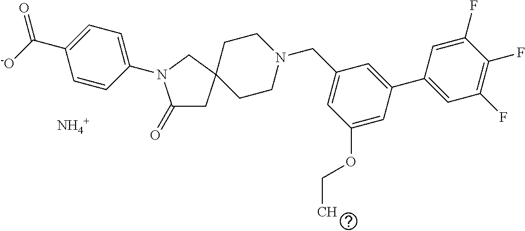 | 538 |
| 116 | 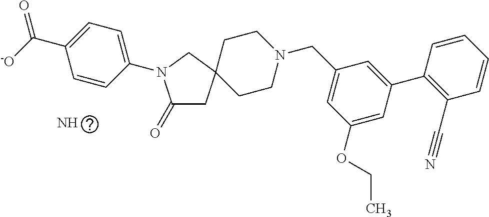 | 509 |
| 117 | 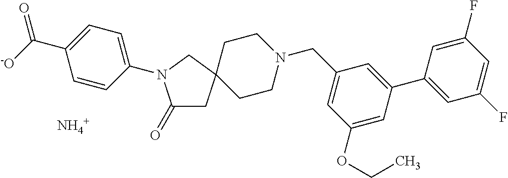 | 520 |
| 118 | 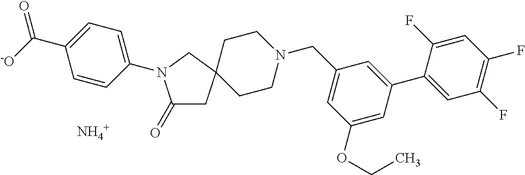 | 538 |
| 119 | 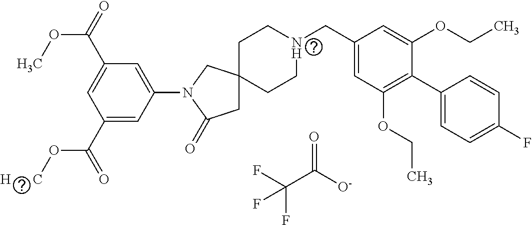 | 618 |
| 120 | 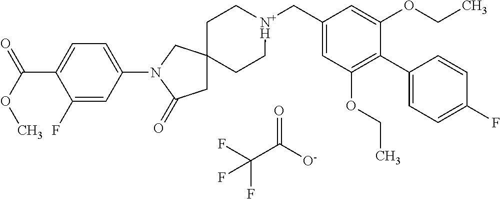 | 578 |
| 121 | 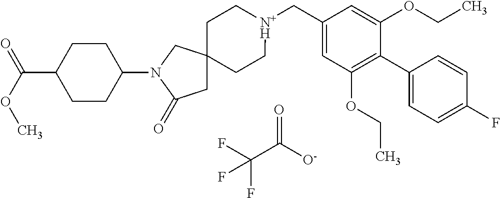 | 566 |
| 122 | 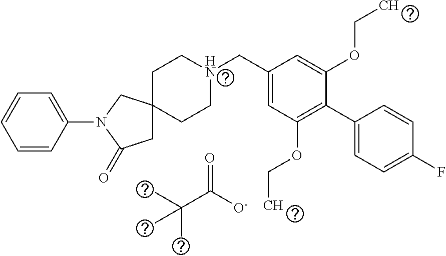 | 502 |
| 123 | 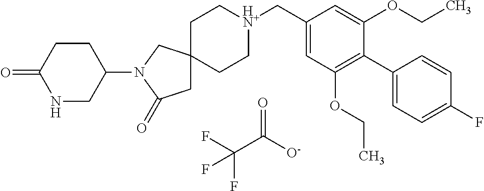 | 523 |
| 124 | 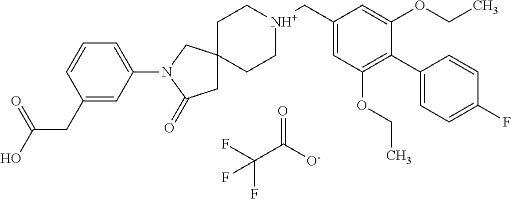 | 560 |
| 125 | 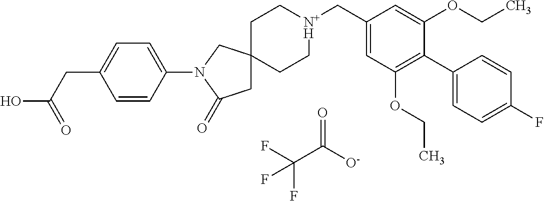 | 560 |
| 126 | 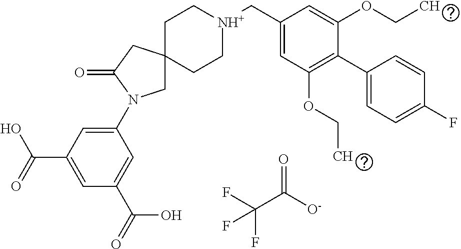 | 590 |
| 127 | 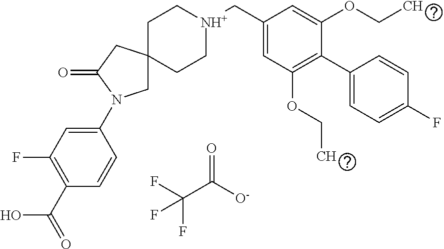 | 564 |
| 130 | 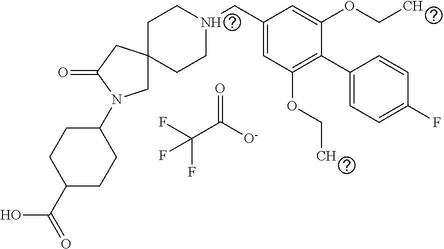 | 552 |
| 131 | 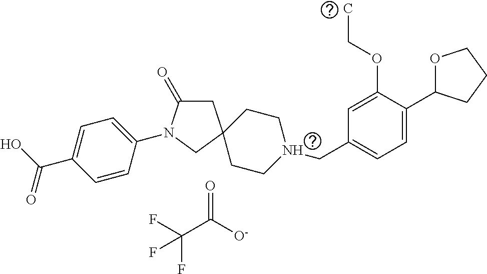 | 478 |
| 132 | 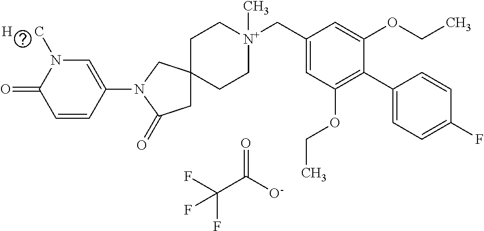 | 548 |
| 133 | 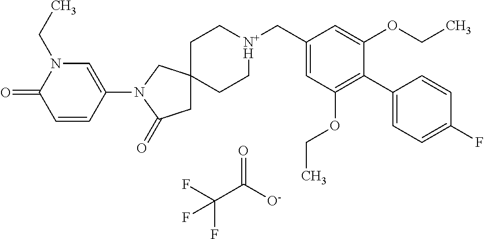 | 547 |
| 134 | 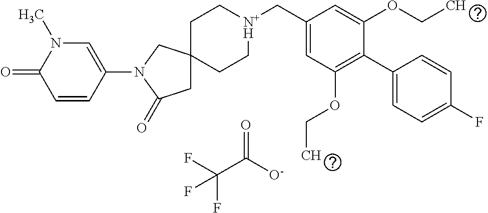 | 533 |
| 135 | 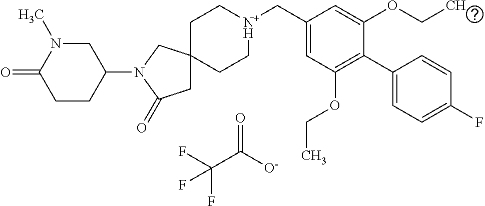 | 537 |
| 136 | 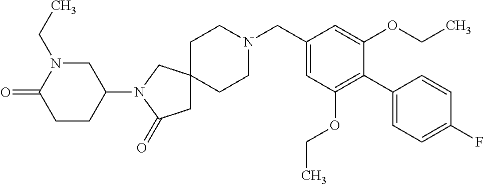 | 551 |
| 137 | 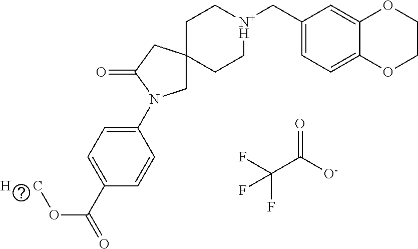 | 436 |
| 138 | 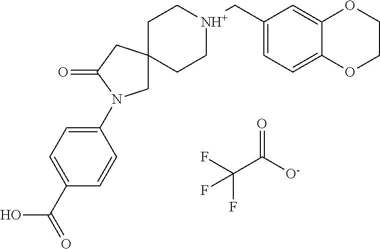 | 422 |
| 139 | 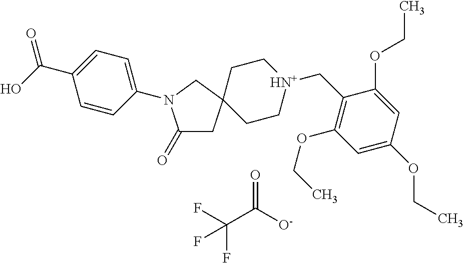 | 496 |
| 140 | 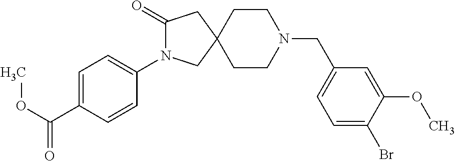 | 487 |
| 141 | 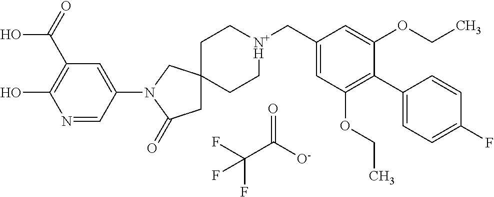 | 563 |
| 142 | 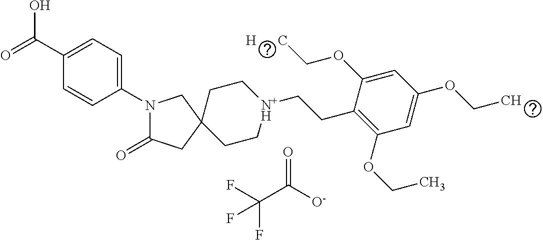 | 510 |
| 143 | 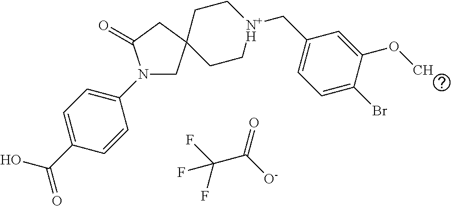 | 473 |
| 144 | 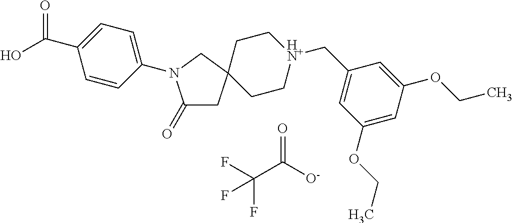 | 452 |
| 145 | 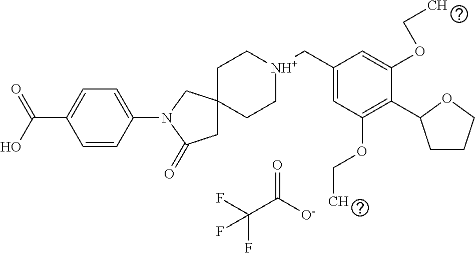 | 522 |
| 146 | 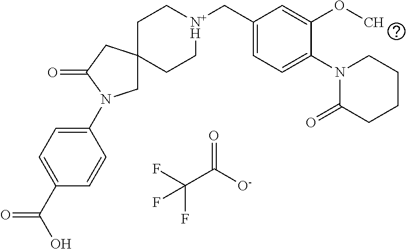 | 491 |
| 147 | 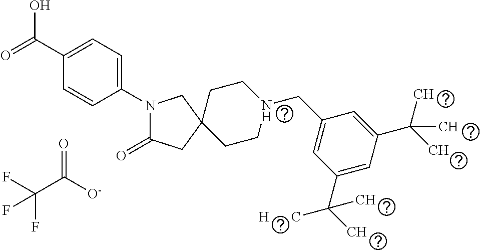 | 476 |
| 148 | 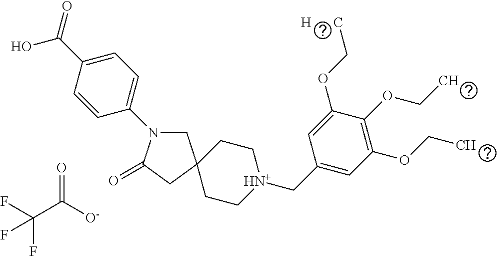 | 496 |
| 149 | 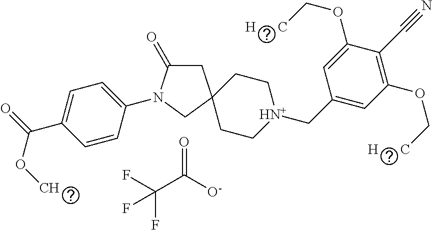 | 491 |
| 150 | 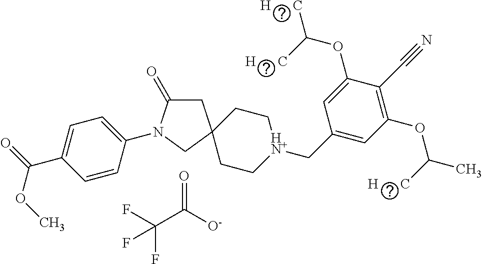 | 519 |
| 151 | 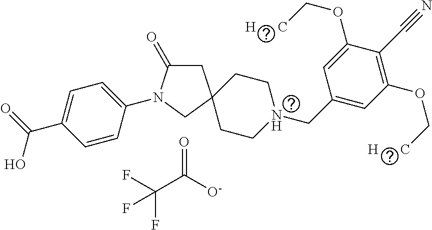 | 477 |
| 152 | 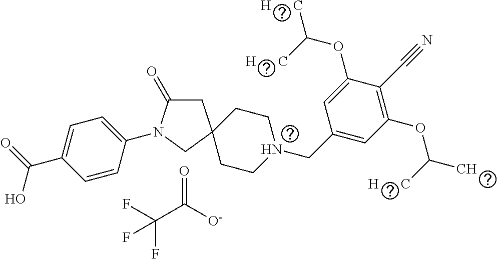 | 505 |
| 153 | 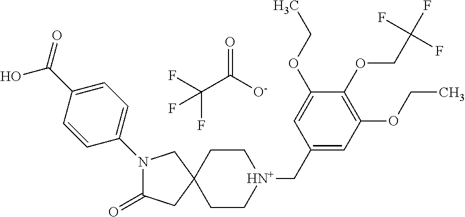 | 550 |
| 154 | 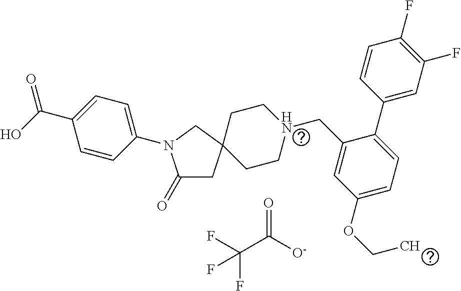 | 520 |
| 155 | 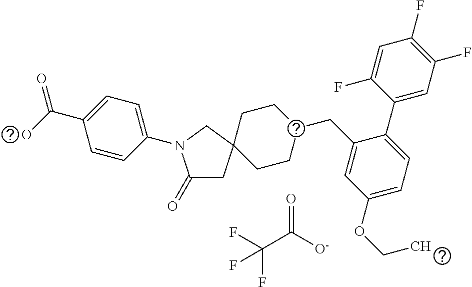 | 538 |
| 156 | 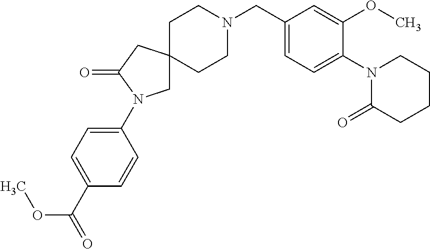 | 505 |
| 157 | 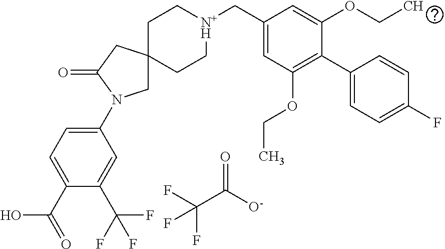 | 614 |
| 158 | 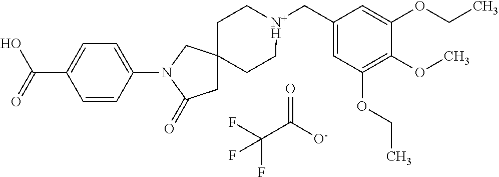 | 482 |
| 159 | 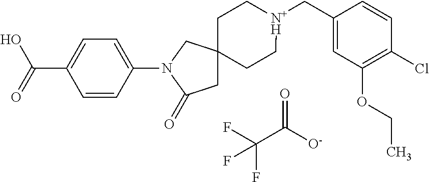 | 442 |
| 160 | 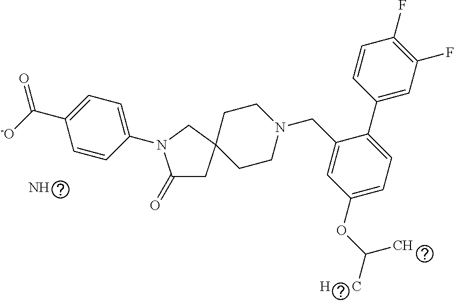 | 534 |
| 161 | 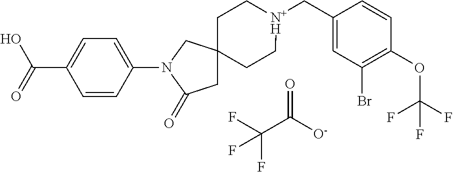 | 527 |
| 162 | 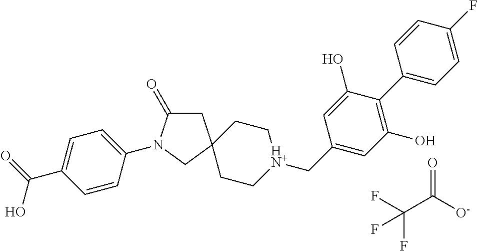 | 490 |
| 163 | 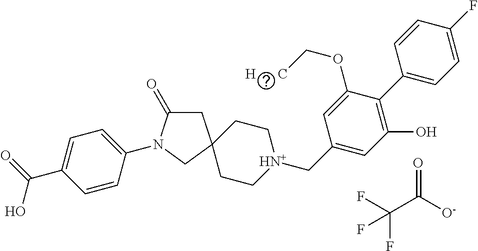 | 518 |
| 163 | 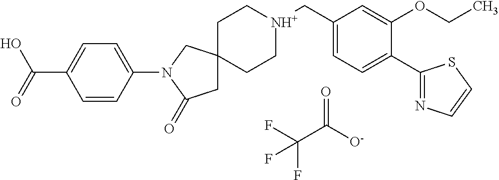 | 491 |
| 164 | 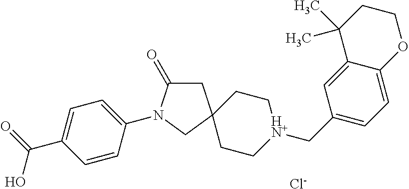 | 448 |
| 165 | 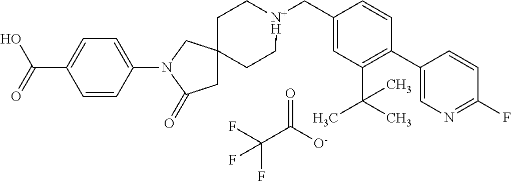 | 515 |

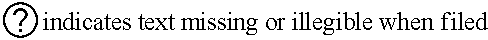

### Biological Assays

SSTR5 antagonists can be identified using SSTR5 and nucleic acid encoding for SSTR5. Suitable assays include detecting compounds competing with a SSTR5 agonist for binding to SSTR5 and determining the functional effect of compounds on a SSTR5 cellular or physiologically relevant activity. SSTR5 cellular activities include cAMP phospholipase C increase, tyrosine phsophatases increase, endothelial nitric oxide synthase (eNOS) decrease, K⁺ channel increase, Na⁺/H⁺ exchange decrease, and ERK decrease. (Lahlou et al., Ann. N.Y. Aced. Sci. 1014:121-131, 2004.) Functional activity can be determined using cell lines expressing SSTR5 and determining the effect of a compound on one or more SSTR5 activities (e.g., Poitout et al., J. Med. Chem. 44:29900-3000, (2001); Hocart et al., J. Med. Chem., 41:1146-1154, (1998); J. Med. Chem. 50, 6292-6295 (2007) and J. Med. Chem. 50, 6295-6298 (2007)).

SSTR5 binding assays can be performed by labeling somatostatin and determining the ability of a compound to inhibit somatostatin binding. (Poitout et al., J. Med. Chem. 44:29900-3000, (2001); Hocart et al., J. Med. Chem. 41:1146-1154, (1998); J. Med. Chem. 50, 6292-6295 (2007) and J. Med. Chem. 50, 6295-6298 (2007)). Additional formats for measuring binding of a compound to a receptor are well-known in the art.

A physiologically relevant activity for SSTR5 inhibition is stimulating insulin secretion. Stimulation of insulin secretion can be evaluated in vitro or in vivo.

Antagonists can be characterized based on their ability to bind to SSTR5 (Ki) and effect SSTR5 activity (IC₅₀), and to selectively bind to SSTR5 and selectively affect SSTR5 activity. Preferred antagonists strongly and selectively bind to SSTR5 and inhibit SSTR5 activity. Ki can be measured as described by Poitout et al., J. Med. Chem. 44:29900-3000, (2001) and described herein.

A selective SSTR5 antagonist binds SSTR5 at least 10 times stronger than it binds SSTR1, SSTR2, SSTR3, and SSTR4. In different embodiments concerning selective SSTR5 binding, the antagonist binds to each of SSTR1, SSTR2, SSTR3, and SSTR4 with a Ki greater than 1000 nM, or preferably greater than 2000 nM and/or binds SSTR5 at least 40 times, more preferably at least 100 times, or more preferably at least 500 times, greater than it binds to SSTR1, SSTR2, SSTR3, and SSTR4.

IC₅₀ can be determined by measuring inhibition of somatostatin-14 or somatostatin-28 induced reduction of cAMP accumulation due to forskolin (1 μM) in CHO-K1 cells expressing SSTR5, as described by Poitout et al., J. Med. Chem. 44:29900-3000, (2001).

### SSTR Binding Assays:

The receptor-ligand binding assays of all 5 subtype of SSTRs were performed with membranes isolated from Chinese hamster ovary (CHO)-K1 cells stably expressing the cloned human somatostatin receptors in 96-well format as previous reported. (Yang et al. PNAS 95:10836-10841, (1998), Birzin et al. Anal. Biochem. 307:159-166, (2002)).

The stable cell lines for SSTR1-SSTR5 were developed by stably transfecting with DNA for all five SSTRs using Lipofectamine. Neomycin-resistant clones were selected and maintained in medium containing 400 μg/mL G418 (Rohrer et al. Science 282:737-740, (1998)). Binding assays were performed using (3-¹²⁵I-Tyr11)-SRIF-14 or (3-¹²⁵I-Tyr11)-SRIF-28 as the radioligand (used at 0.1 nM) and The Packard Unifilter assay plate. The assay buffer consisted of 50 mM TrisHCl (pH 7.8) with 1 mM EGTA, 5 mM MgCl₂, leupeptin (10 μg/mL), pepstatin (10 μg/mL), bacitracin (200 μg/mL), and aprotinin (0.5 μg/mL). CHO-K1 cell membranes, radiolabeled somatostatin, and unlabeled test compounds were resuspended or diluted in this assay buffer. Unlabeled test compounds were examined over a range of concentrations from 0.01 nM to 10,000 nM. The Kᵢ values for compounds were determined as described by Cheng and Prusoff Biochem Pharmacol. 22:3099-3108 (1973).

The compounds of the present invention, particularly the compounds of Examples 1-90, were tested in the SSTR5 binding assay and found to have Kᵢ values in the range of 0.1 nM to 1 uM against SSTR5, as shown in Table 2, and were found to have IQ values greater than 100 nM against SSTR1, SSTR2, SSTR3, and SSTR4 receptors. Preferred compounds of the present invention were found to have Kᵢ values in the range of 0.1 nM to 100 nM against SSTR5, and Kᵢ values greater than 100 nM against SSTR1, SSTR2, SSTR3, and SSTR4 receptors. More preferred compounds of the present invention were found to have Kᵢ values in the range of 0.1 nM to 10 nM against SSTR5, and Kᵢ values greater than 100 nM against SSTR1, SSTR2, SSTR3, and SSTR4 receptors.

### Functional Assay to Assess the Inhibition of SSTR5 Mediated Cyclic AMP Production:

The effects of compounds that bind to human and murine SSTR5 with various affinities on the functional activity of the receptor were assessed by measuring cAMP production in the presence of Forskolin (FSK) alone or FSK plus SS-28 in SSTR5 expressing CHO cells. FSK acts to induce cAMP production in these cells by activating adenylate cyclases, whereas SS-28 suppresses cAMP production in the SSTR5 stable cells by binding to SSTR5 and the subsequent inhibition of adenylate cyclases via an alpha subunit of GTP-binding protein (Gai).

To measure the agonism activity of the compounds, human or mouse SSTR5 stable CHO cells were pre-incubated with the compounds for 15 min, followed by a one-hour incubation of the cells with 5 μM FSK (in the continuous presence of the compounds). The amount of cAMP produced during the incubation was quantified with the Lance cAMP assay kit (PerkinElmer, CA) according to the manufacturer's instruction, as well as, an IC₅₀ value was obtained by an eight-point titration.

The compounds of the present invention, particularly the compounds of Examples 1-90, were tested in the SSTR5 binding assay and found to have cAMP IC₅₀ values in the range of 0.1 nM to 1 μM against SSTR5, as shown in Table 2, and were found to have cAMP IC₅₀ values greater than 100 nM against SSTR1, SSTR2, SSTR3, and SSTR4 receptors. Preferred compounds of the present invention were found to have cAMP IC₅₀ values in the range of 0.1 nM to 100 nM against SSTR5, and IC₅₀ values greater than 100 nM against SSTR1, SSTR2, SSTR3, and SSTR4 receptors. More preferred compounds of the present invention were found to have cAMP IC₅₀ values in the range of 0.1 nM to 10 nM against SSTR5, and IC₅₀ values greater than 100 nM against SSTR1, SSTR2, SSTR3, and SSTR4 receptors.

TABLE 2

| | | | SSTR5 |
|---|---|---|---|
| | | SSTR5 Ki | (cAMP) IC₅₀ |
| Example | Compound | (nM) | (nM) |
| 1 | 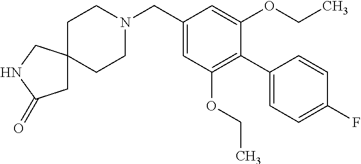 | 21 | 52 |
| 9 | 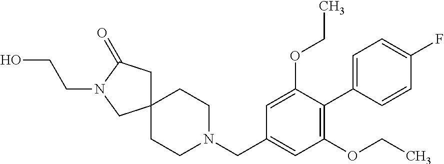 | 71 | 159 |
| 23 | 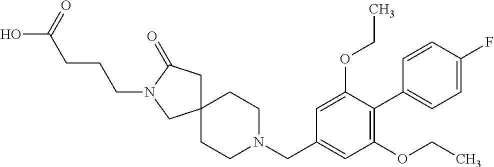 | 53 | 44 |
| 35 | 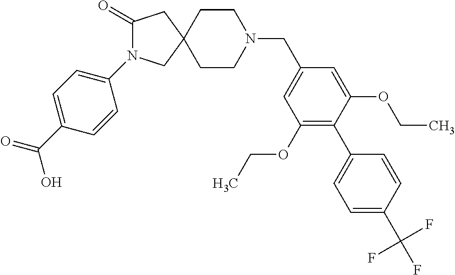 | 0.86 | 0.62 |
| 37 | 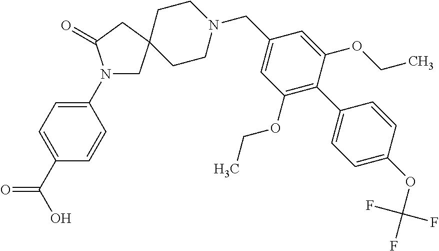 | 0.4853 | 0.6 |
| 16 | 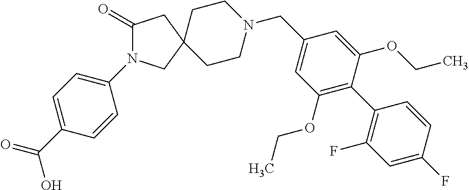 | 0.735 | 0.95 |
| 44 | 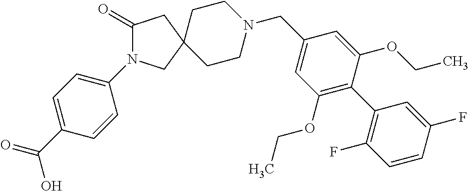 | 1.547 | 0.28 |
| 45 | 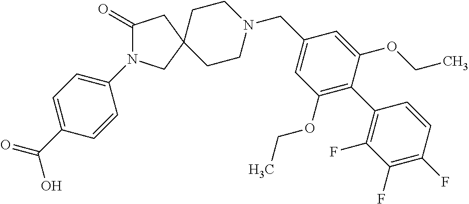 | 0.84 | 1.5 |
| 8 | 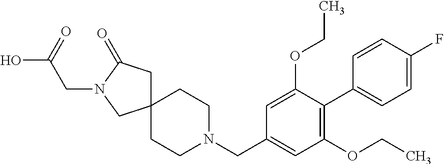 | 190 | 159 |
| 49 | 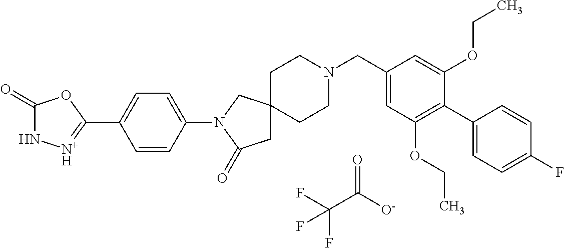 | 8.9 | 34 |
| 52 | 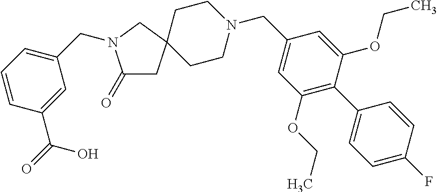 | 67 | 21 |
| 53 | 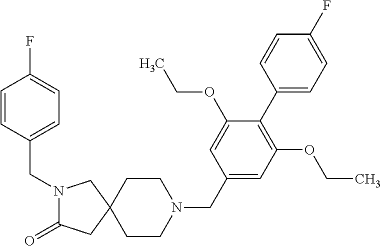 | 3.9 | 8.6 |
| 57 | 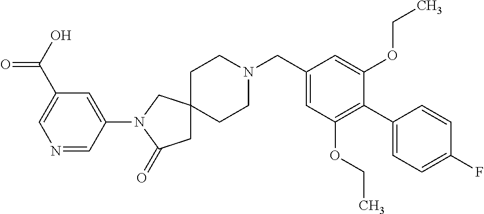 | 10 | 3.6 |
| 59 | 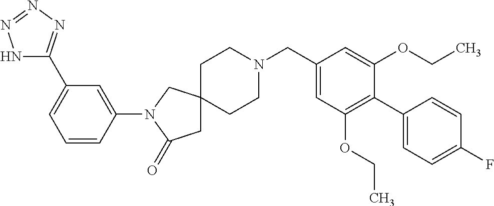 | 3.6 | 4.7 |
| 60 | 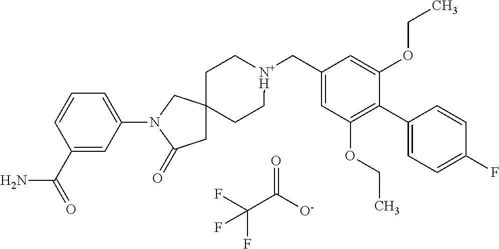 | 4.1 | 16 |
| 3 | 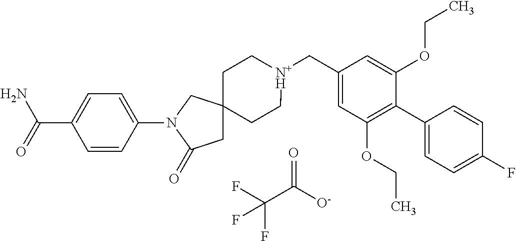 | 8.7 | 33 |
| 61 | 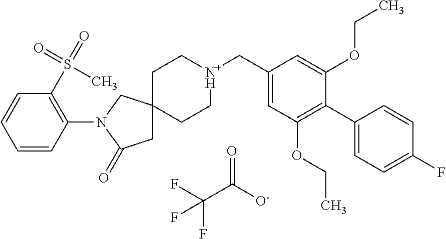 | 2 | 13 |
| 65 | 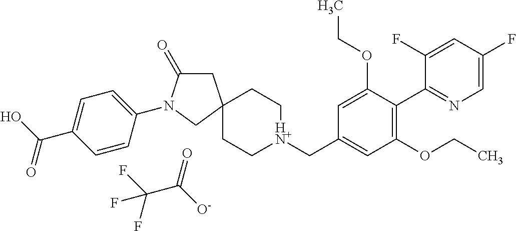 | 4.5 | 3.6 |
| 66 | 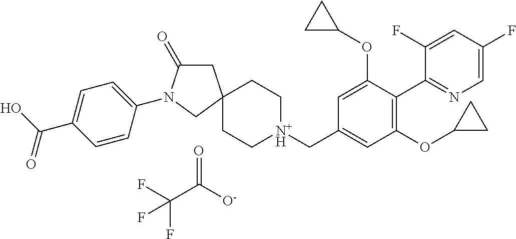 | 4.5 | 1.8 |
| 2 | 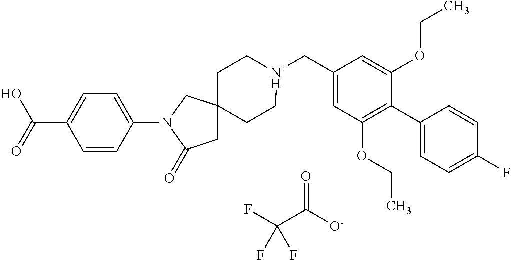 | 1.5 | 1.8 |
| 74 | 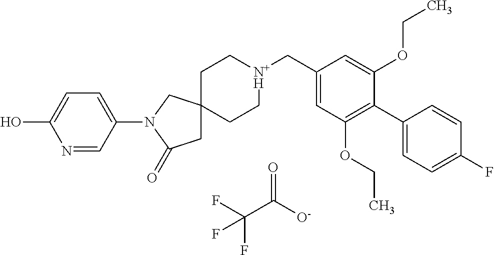 | 3.1 | 3.1 |
| 78 | 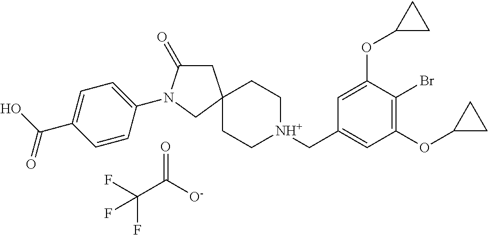 | 9.2 | 5.6 |
| 81 | 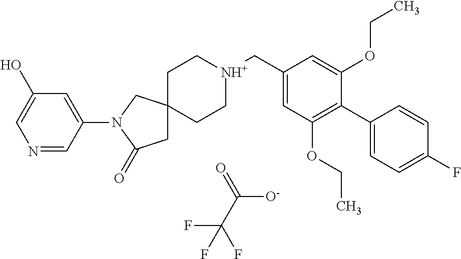 | 3.7 | 7.9 |
| 84 | 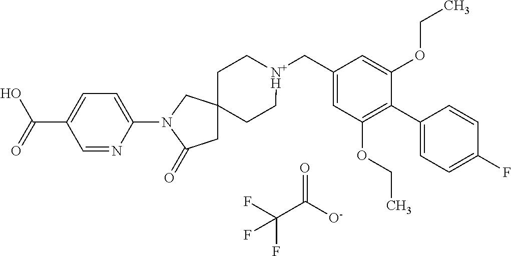 | 7.7 | 4.7 |
| 85 | 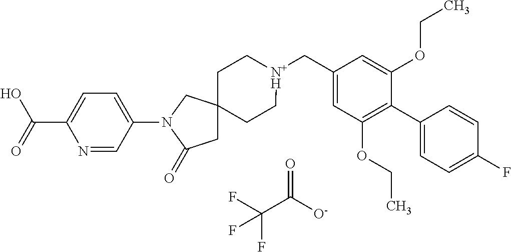 | 5.27 | 6.17 |
| 87 | 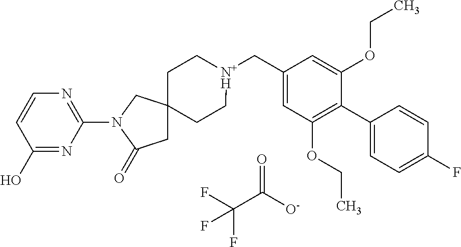 | 337 | 64 |
| 6 | 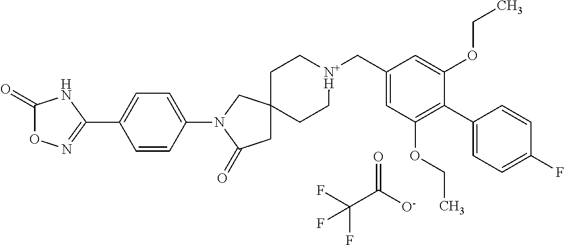 | 1 | 1.8 |
| 7 | 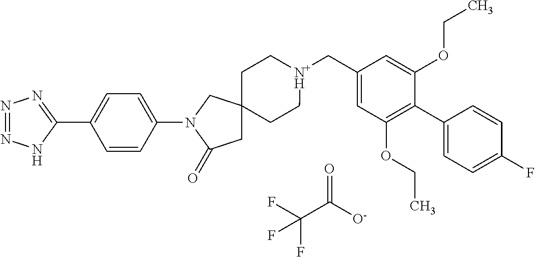 | 0.76 | 1.3 |
| 5 | 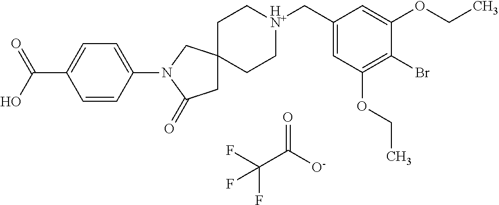 | 7.5 | 3.9 |
| 94 | 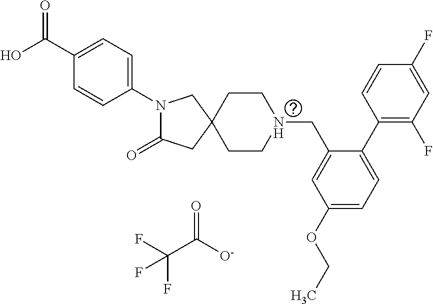 | 11 | 7.3 |
| 97 | 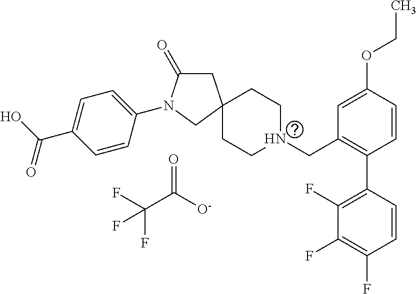 | 7.5 | 1.8 |
| 101 | 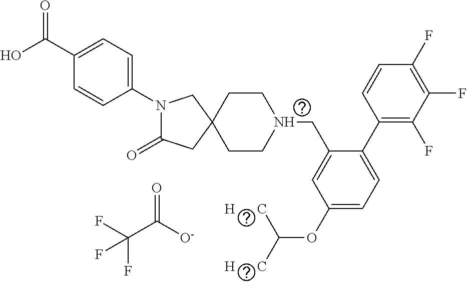 | 4.2 | 4.5 |
| 103 | 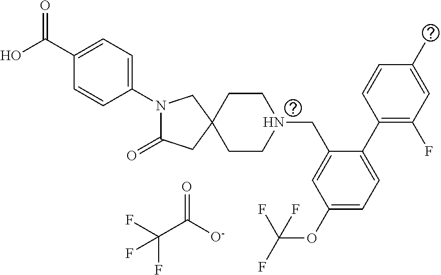 | 12 | 1900 |
| 107 | 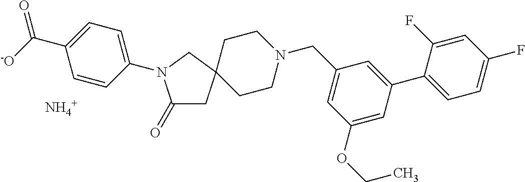 | 40 | 36 |
| 148 | 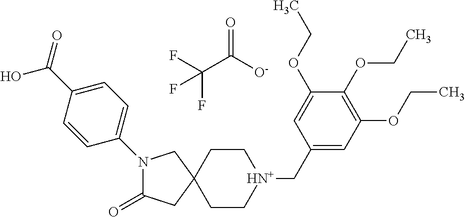 | 157 | 82 |
| 152 | 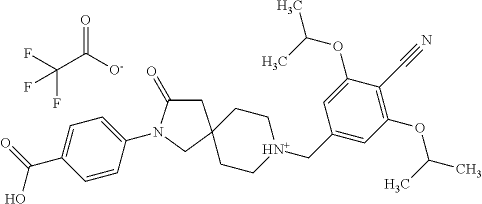 | 14 | 15 |
| 153 | 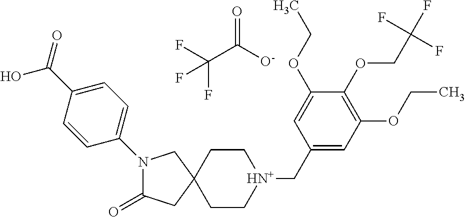 | 55 | 42 |
| 159 | 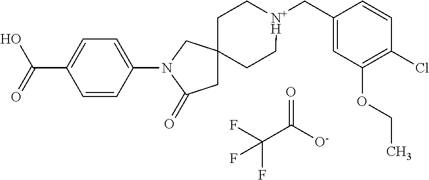 | 130 | 61 |
| 162 | 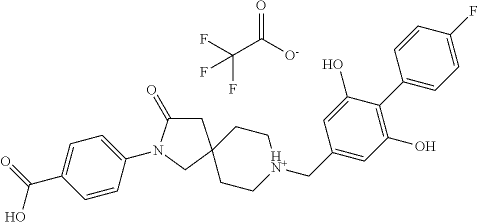 | 1600 | >1000 |
| 163 | 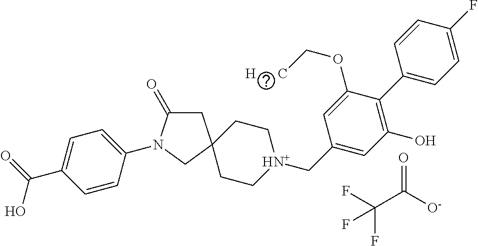 | 42 | 59 |

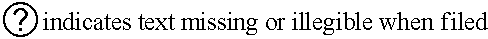

### Enhancement of Glucose Dependent Insulin Secretion (GDIS) by SSTR3 Antagonists in Isolated Mouse Islet Cells:

Pancreatic islets of Langerhans were isolated from the pancreas of normal C57BL/6J mice (Jackson Laboratory, Maine) by collagenase digestion and discontinuous Ficoll gradient separation, a modification of the original method of Lacy and Kostianovsky (Lacy et al., Diabetes 16:35-39, 1967). The islets were cultured overnight in RPMI 1640 medium (11 mM glucose) before GDIS assay.

To measure GDIS, islets were first preincubated for 30 minutes in the Krebs-Ringer bicarbonate (KRB) buffer with 2 mM glucose (in petri dishes). The KRB medium contains 143.5 mM Na⁺, 5.8 mM K⁺, 2.5 mM Ca²⁺, 1.2 mM Mg²⁺, 124.1 mM Cl⁻, 1.2 mM PO₄³⁻, 1.2 mM SO₄²⁺, 25 mM CO₃²⁻, 2 mg/mL bovine serum albumin (pH 7.4). The islets were then transferred to a 96-well plate (one islet/well) and incubated at 37° C. for 60 minutes in 200 μl of KRB buffer with 2 or 16 mM glucose, and other agents to be tested such as octreotide and a SST3 antagonist. (Zhou et al., J. Biol. Chem. 278:51316-51323, 2003.) Insulin was measured in aliquots of the incubation buffer by ELISA with a commercial kit (ALPCO Diagnostics, Windham, N.H.).

### Glucose Tolerance Test in Mice:

Male C57BL/6N mice (7-12 weeks of age) are housed 10 per cage and given access to normal diet rodent chow and water ad libitum. Mice are randomly assigned to treatment groups and fasted 4 to 6 h. Baseline blood glucose concentrations are determined by glucometer from tail nick blood. Animals are then treated orally with vehicle (0.25% methylcellulose) or test compound. Blood glucose concentration is measured at a set time point after treatment (t=0 min) and mice are then challenged with dextrose intraperitoneally-(2-3 g/kg) or orally (3-5 g/kg). One group of vehicle-treated mice is challenged with saline as a negative control. Blood glucose levels are determined from tail bleeds taken at 20, 40, 60 minutes after dextrose challenge. The blood glucose excursion profile from t=0 to t=60 min is used to integrate an area under the curve (AUC) for each treatment. Percent inhibition values for each treatment are generated from the AUC data normalized to the saline-challenged controls. A similar assay may be performed in rats. Compounds of the present invention are active after an oral dose in the range of 0.1 to 100 mg/kg.

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size 0 hard gelatin capsule.

As a second specific embodiment of an oral composition of a compound of the present invention, 100 mg of the compound of any of the Examples, microcrystalline cellulose (124 mg), croscarmellose sodium (8 mg), and anhydrous unmilled dibasic calcium phosphate (124 mg) are thoroughly mixed in a blender; magnesium stearate (4 mg) and sodium stearyl fumarate (12 mg) are then added to the blender, mixed, and the mix transferred to a rotary tablet press for direct compression. The resulting tablets are unsubstituted or film-coated with Opadry® II for taste masking.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

What is claimed is:

1. A compound of structural formula I: 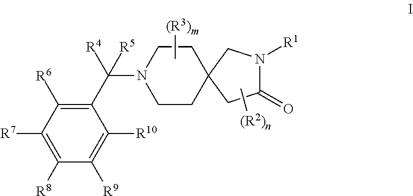
or a pharmaceutically acceptable salt thereof, wherein
R¹ is selected from the group consisting of:
(1) hydrogen,
(2) —C₁₋₁₀alkyl,
(3) —(CH₂)ₛOR^{e},
(4) —(CH₂)ₛNR^{c}R^{d};
(5) —(CH₂)ₛOC₁₋₁₀alkyl,
(6) —(CH₂)ᵣCO₂H,
(7) —(CH₂)ᵣCO₂R^{e};
(8) —(CH₂)ᵣCONR^{c}R^{d},
(9) —(CH₂)ᵣCOR^{e},
(10) —S(O)_{q}C₁₋₁₀alkyl,
(11) —S(O)_{q}(CH₂)ₚaryl,
(12) —S(O)_{q}(CH₂)ₚcycloalkyl,
(13) —S(O)_{q}(CH₂)ₚcycloheteroalkyl,
(14) —S(O)_{q}(CH₂)ₚheteroaryl,
(15) —(CH₂)ₚC₃₋₁₀ cycloalkyl,
(16) —(CH₂)ₚC₂₋₁₀ cycloheteroalkyl,
(17) —(CH₂)ₚaryl, and
(18) —(CH₂)ₚheteroaryl,
wherein CH₂, alkyl, cycloalkyl, cycloheteroalkyl, aryl and heteroaryl are unsubstituted or substituted with one, two or three substituents independently selected from R^{a};
R² is selected from the group consisting of:
(1) hydrogen,
(2) C₁₋₆alkyl, and
(3) —OC₁₋₆alkyl;
R³ is selected from the group consisting of:
(1) hydrogen, and
(2) C₁₋₆alkyl;
R⁴ is selected from the group consisting of:
(1) hydrogen, and
(2) —C₁₋₆ alkyl;
R⁵ is selected from the group consisting of:
(1) hydrogen, and
(2) —C₁₋₆ alkyl,
or R⁴ and R⁵ together with the atom to which they are attached form a cycloalkyl ring with 3 to 7 carbon atoms;
R⁶ is selected from the group consisting of:
(1) hydrogen,
(2) halogen,
(3) —C₁₋₁₀ alkyl,
(4) —OC₁₋₁₀ alkyl,
(5) aryl, and
(6) heteroaryl;
R⁷ is selected from the group consisting of:
(1) hydrogen,
(2) —C₁₋₁₀alkyl,
(3) —C₃₋₁₀cycloalkyl,
(4) —OH,
(5) —O—C₁₋₁₀alkyl,
(6) —O—C₃₋₁₀cycloalkyl,
(7) —O—C₂₋₁₀cycloheteroalkyl,
(8) —O-aryl,
(9) —O-heteroaryl,
(10) —NR^{c}S(O)ₜR^{e},
(11) halogen,
(12) —NR^{c}R^{d},
(13) —CN,
(14) —NR^{c}C(O)R^{e},
(15) —OCF₃,
(16) —OCHF₂,
(17) C₂₋₁₀cycloheteroalkyl,
(18) aryl, and
(19) heteroaryl,
wherein alkyl, cycloalkyl, cycloheteroalkyl, aryl and heteroaryl are unsubstituted or substituted with 1, 2 or 3 halogens;
R⁸ is selected from the group consisting of:
(1) —OC₁₋₆alkyl,
(2) —NR^{c}S(O)ᵤR^{e},
(3) halogen,
(4) —S(O)ᵤR^{e},
(5) —S(O)ᵤNR^{c}R^{d},
(6) —NR^{c}R^{d},
(7) —CN,
(8) —C(O)NR^{c}R^{d},
(9) —NR^{c}C(O)R^{e},
(10) —NR^{c}C(O)OR^{e},
(11) —NR^{c}C(O)NR^{c}R^{d},
(12) —OCF₃,
(13) —OCHF₂,
(14) C₃₋₁₀cycloheteroalkyl,
(15) C₁₋₁₀alkyl,
(16) C₃₋₆cycloalkyl,
(17) aryl, and
(18) heteroaryl,
wherein alkyl, cycloalkyl, cycloheteroalkyl, aryl and heteroaryl are unsubstituted or substituted with one, two or three substituents independently selected from R^{b};
each R⁹ is selected from the group consisting of:
(1) hydrogen,
(2) —C₁₋₁₀alkyl,
(3) —C₃₋₁₀cycloalkyl,
(4) —OH,
(5) —O—C₁₋₁₀alkyl,
(6) —O—C₃₋₁₀cycloalkyl,
(7) —O—C₂₋₁₀cycloheteroalkyl,
(8) —O-aryl,
(9) —O-heteroaryl,
(10) —NR^{c}S(O)ₜR^{e};
(11) halogen,
(12) —NR^{c}R^{d},
(13) —CN,
(14) —NR^{c}C(O)R^{e},
(15) —OCF₃,
(16) —OCHF₂,
(17) C₂₋₁₀cycloheteroalkyl,
(18) aryl, and
(19) heteroaryl,
wherein alkyl, cycloalkyl, cycloheteroalkyl, aryl and heteroaryl are unsubstituted or substituted with 1, 2 or 3 halogens;
R¹⁰ is selected from the group consisting of:
(1) hydrogen,
(2) halogen,
(3) —C₁₋₁₀ alkyl, and
(4) —OC₁₋₁₀alkyl;
each R^{a} is independently selected from the group consisting of:
(1) —C₁₋₆alkyl,
(2) —CF₃,
(3) —OH,
(4) —OC₁₋₆alkyl,
(5) —OCF₃,
(6) —OCHF₂,
(7) —OCH₂F,
(8) halogen,
(9) —S(O)ᵥR^{e},
(10) —S(O)ᵥNR^{c}R^{d},
(11) —NR^{c}S(O)ᵥR^{e},
(12) —NO₂,
(13) —NR^{c}R^{d},
(14) —C(O)R^{e},
(15) —CO₂H,
(16) —CO₂R^{e},
(17) —OC(O)R^{e},
(18) —CN,
(19) —C(O)NR^{c}R^{d},
(20) —NR^{c}C(O)R^{e},
(21) —NR^{c}C(O)OR^{e},
(22) —NR^{c}C(O)NR^{c}R^{d},
(23) C₃₋₁₀ cycloalkyl,
(24) C₂₋₁₀ cycloheteroalkyl,
(25) aryl, and
(26) heteroaryl,
wherein alkyl, cycloalkyl, cycloheteroalkyl, aryl and heteroaryl are unsubstituted or substituted with 1 or 2 substituents selected from oxo, C₁₋₆alkyl, —CO₂H, —NH₂, NH(C₁₋₆alkyl), and NH(C₁₋₆alkyl)₂;
each R^{b} is independently selected from the group consisting of:
(1) —CN,
(2) halogen,
(3) —CF₃,
(4) —OCF₃,
(5) —C₁₋₆alkyl,
(6) —OC₁₋₆alkyl,
(7) aryl, and
(8) heteroaryl;
each R^{c} is independently selected from the group consisting of:
(1) hydrogen, and
(2) C₁₋₆alkyl;
each R^{d} is independently selected from the group consisting of:
(1) hydrogen, and
(2) C₁₋₆alkyl;
each R^{e} is independently selected from the group consisting of:
(1) C₁₋₆ alkyl,
(2) C₃₋₁₀cycloalkyl,
(3) C₂₋₁₀ cycloheteroalkyl,
(4) aryl, and
(5) heteroaryl;
m is 0, 1, 2, 3 or 4;
n is 0, 1 or 2;
p is 0, 1, 2, 3, 4 or 5;
q, t, u and v are 1 or 2;
r is 1, 2, 3, 4 or 5; and
s is 2, 3 or 4.

2. The compound of claim 1 wherein R², R³, R⁴, R⁵, R⁶ and R¹⁰ are each hydrogen; or a pharmaceutically acceptable salt thereof.

3. The compound of claim 2 wherein R⁷ and R⁹ are independently selected from the group consisting of:
(1) —O—C₁₋₁₀alkyl, and
(2) —O—C₃₋₁₀cycloalkyl;
or a pharmaceutically acceptable salt thereof.

4. The compound of claim 2 wherein R⁷ and R⁹ are independently selected from the group consisting of:
(1) —O—CH₂CH₃, and
(2) —O-cyclopropyl;
or a pharmaceutically acceptable salt thereof.

5. The compound of claim 2 wherein R⁸ is selected from the group consisting of
(1) halogen,
(2) aryl, and
(3) heteroaryl,
wherein aryl and heteroaryl are unsubstituted or substituted with one, two or three substituents independently selected from R^{b};
or a pharmaceutically acceptable salt thereof.

6. The compound of claim 2 wherein R⁸ is selected from the group consisting of:
(1) phenyl, and
(2) pyridine,
wherein phenyl and pyridine are unsubstituted or substituted with one or two substituents independently selected from R^{b};
or a pharmaceutically acceptable salt thereof.

7. The compound of claim 2 wherein R¹ is selected from the group consisting of:
(1) hydrogen,
(2) —(CH₂)ₛOH,
(3) —(CH₂)ᵣCO₂H,
(4) —(CH₂)ᵣCO₂C₁₋₁₀alkyl,
(5) —(CH₂)ᵣCONR^{c}R^{d},
(6) —S(O)_{q}(CH₂)ₚaryl,
(7) —(CH₂)ₚaryl, and
(8) —(CH₂)ₚheteroaryl,
wherein CH₂, alkyl, aryl and heteroaryl are unsubstituted or substituted with one, two or three substituents independently selected from R^{a};
or a pharmaceutically acceptable salt thereof.

8. The compound of claim 2 wherein R¹ is selected from the group consisting of:
(1) hydrogen,
(2) phenyl, and
(3) pyridine,
wherein phenyl and pyridine are unsubstituted or substituted with one substituent independently selected from R^{a};
or a pharmaceutically acceptable salt thereof.

9. The compound of claim 2 wherein R¹ is selected from the group consisting of:
(1) phenyl, and
(2) pyridine,
wherein phenyl and pyridine are substituted with one substituent independently selected from R^{a};
or a pharmaceutically acceptable salt thereof.

10. The compound of claim 2 wherein each R^{a} is independently selected from the group consisting of:
(1) —OH,
(2) —CN,
(3) —OC₁₋₆alkyl,
(4) halogen,
(5) —S(O)₂C₁₋₆alkyl,
(6) —CO₂H,
(7) —CO₂C₁₋₆alkyl,
(8) —C(O)NRCR^{d}, and
(9) heteroaryl,
wherein alkyl and heteroaryl are unsubstituted or substituted with 1 or 2 substituents selected from oxo, C₁₋₆alkyl, —CO₂H, —NH₂, NH(C₁₋₆alkyl), and NH(C₁₋₆alkyl)₂;
or a pharmaceutically acceptable salt thereof.

11. The compound of claim 2 wherein each R^{a} is independently selected from the group consisting of:
(1) —CO₂H,
(2) —C(O)NR^{c}R^{d}, and
(3) heteroaryl,
wherein heteroaryl is unsubstituted or substituted with 1 or 2 substituents selected from oxo;
or a pharmaceutically acceptable salt thereof.

12. The compound of claim 1 wherein:
R¹ is selected from the group consisting of
(1) hydrogen,
(2) —(CH₂)ₛOH,
(3) —(CH₂)ᵣCO₂H,
(4) —(CH₂)ᵣCO₂C₁₋₁₀alkyl,
(5) —(CH₂)ᵣCONR^{c}R^{d},
(6) —S(O)_{q}(CH₂)ₚaryl,
(7) —(CH₂)ₚaryl, and
(8) —(CH₂)ₚheteroaryl,
wherein CH₂, alkyl, aryl and heteroaryl are unsubstituted or substituted with one, two or three substituents independently selected from R^{a};
R², R³, R⁴, R⁵, R⁶ and R¹⁰ are each hydrogen;
R⁷ and R⁹ are independently selected from the group consisting of:
(1) —O—C₁₋₁₀alkyl, and
(2) —O—C₃₋₁₀cycloalkyl;
R⁸ is selected from the group consisting of:
(1) halogen,
(2) aryl, and
(3) heteroaryl,
wherein aryl and heteroaryl are unsubstituted or substituted with one, two or three substituents independently selected from R^{b};
each R^{a} is independently selected from the group consisting of:
(1) —OH,
(2) —CN,
(3) —OC₁₋₆alkyl,
(4) halogen,
(5) —S(O)₂C₁₋₆alkyl,
(6) —CO₂H,
(7) —CO₂C₁₋₆alkyl,
(8) —C(O)NR^{c}R^{d}, and
(9) heteroaryl,
wherein alkyl and heteroaryl is unsubstituted or substituted with 1 or 2 substituents selected from oxo, C₁₋₆alkyl, —CO₂H, —NH₂, NH(C₁₋₆alkyl), and NH(C₁₋₆alkyl)₂; and
each R^{b} is independently selected from the group consisting of:
(1) —CN,
(2) halogen,
(3) —CF₃,
(4) —OCF₃,
(5) —C₁₋₆alkyl,
(6) —OC₁₋₆alkyl, and
(7) —C(O)NR^{c}R^{d};
or a pharmaceutically acceptable salt thereof.

13. The compound of claim 1 wherein:
R¹ is selected from the group consisting of:
(1) hydrogen,
(2) phenyl, and
(3) pyridine,
wherein phenyl and pyridine are unsubstituted or substituted with one substituent independently selected from R^{a};
R², R³, R⁴, R⁵, R⁶ and R¹⁰ are each hydrogen;
R⁷ and R⁹ are independently selected from the group consisting of:
(1) —O—CH₂CH₃, and
(2) —O-cyclopropyl;
R⁸ is selected from the group consisting of:
(1) phenyl, and
(2) pyridine,
wherein phenyl and pyridine are unsubstituted or substituted with one or two substituents independently selected from R^{b};
each R^{a} is independently selected from the group consisting of:
(1) —CO₂H,
(2) —C(O)NH₂,
(3) tetrazole, and
(4) oxo-dihydro-oxadiazole;
each R^{b} is independently selected from the group consisting of halogen;
or a pharmaceutically acceptable salt thereof.

14. The compound of claim 2 selected from the group consisting of: 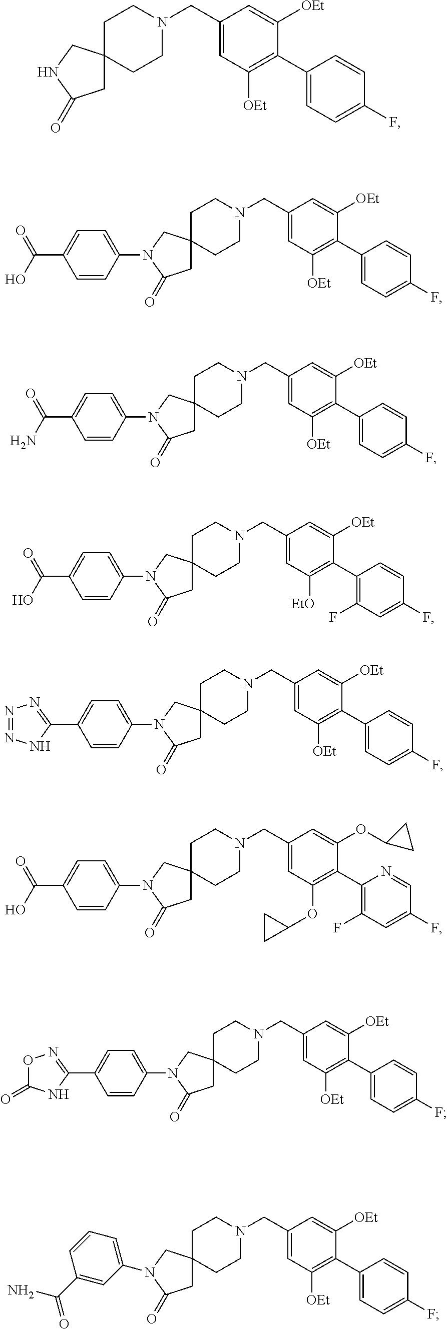 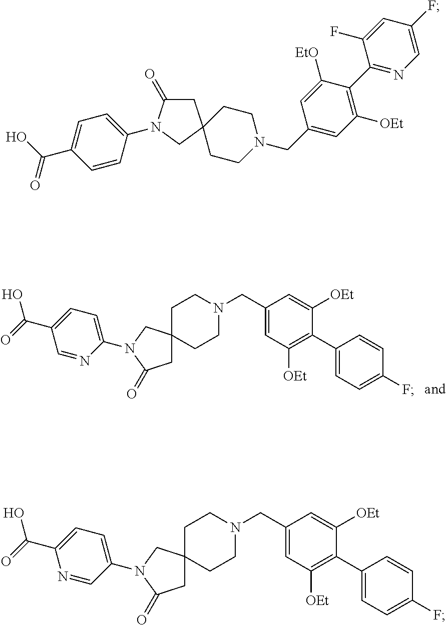
or a pharmaceutically acceptable salt thereof.

15. The compound of claim 14 which is: 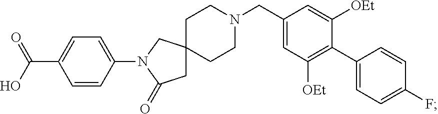
or a pharmaceutically acceptable salt thereof.

16. The compound of claim 14 which is: 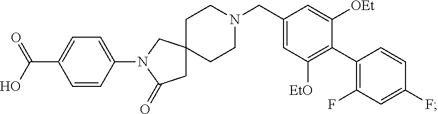
or a pharmaceutically acceptable salt thereof.

17. The compound of claim 14 which is: 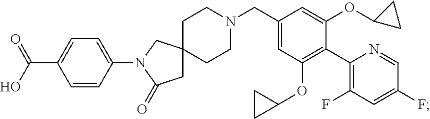
or a pharmaceutically acceptable salt thereof.

18. The compound of claim 14 which is: 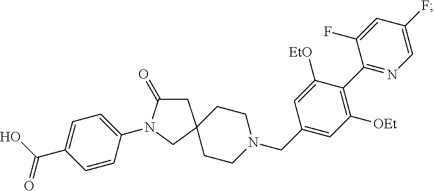
or a pharmaceutically acceptable salt thereof.

19. A pharmaceutical composition comprising a compound of claim 1, or a pharmaceutically acceptable salt thereof, in combination with a pharmaceutically acceptable carrier.

20. Use of a compound of claim 1, or a pharmaceutically acceptable salt thereof, for treating a disorder, condition, or disease responsive to antagonism of the somatostatin subtype receptor 5 in a subject in need thereof.

21. The use of claim 15 wherein said disorder, condition, or disease is selected from the group consisting of: Type 2 diabetes, insulin resistance, a lipid disorder, obesity, metabolic syndrome, depression and anxiety.

22. Use of a compound of claim 1, or a pharmaceutically acceptable salt thereof, for the manufacture of a medicament for treating Type 2 diabetes, insulin resistance, a lipid disorder, obesity, metabolic syndrome, depression and anxiety in a subject in need thereof.
